(19) United States
(12) Patent Application Publication
(10) Pub. No.: US 2014/0296571 A1
(43) Pub. Date: Oct. 2, 2014

(54) Microorganisms And Methods For Producing Propionic Acid

(71) Applicant: The Procter & Gamble Company, (US)

(72) Inventors: Phillip Richard Green, Wyoming, OH (US); Jun Xu, Mason, OH (US); Charles Winston Saunders, Fairfield, OH (US)

(73) Assignee: The Procter & Gamble Company, Cincinnati, OH (US)

(21) Appl. No.: 13/852,036

(22) Filed: Mar. 28, 2013

(51) Int. Cl. C12P 7/52

(52) U.S. Cl. CPC C12P 7/52
USPC 562/606; 435/141

(57) ABSTRACT

This invention relates to microorganisms that convert a carbon source to propionate. The invention provides genetically engineered microorganisms that carry out the conversion, as well as methods for producing propionate by culturing the microorganisms.

### FIELD OF THE INVENTION

This invention relates to microorganisms that convert a carbon source to propionic acid (propanoic acid), which can be produced from glucose using a threonine and a 2-ketobutyrate intermediate, from glucose using a citramalate and a 2-ketobutyrate intermediate, or from glucose using succinyl-CoA and methylmalonyl-CoA intermediates. The invention provides genetically engineered microorganisms that carry out the conversion, as well as methods for producing propionate (propanoate) by culturing the microorganisms or by using isolated enzymes.

### BACKGROUND OF THE INVENTION

Propionic acid is a chemical used directly in food preservation, and as an intermediate in the synthesis of perfumes, herbicides, pharmaceuticals, polymers and other applications (Liu, L., et. al. Critical Reviews in Biotechnology (2012) 1-8). Traditionally, propionic acid is made from ethylene sources by hydrocarboxylation, or the oxidation of propionaldehyde, itself produced by hydroformylation of ethylene. Ethylene itself is a byproduct of oil refining from petroleum (i.e., crude oil) and of natural gas production. Disadvantages associated with traditional propionic acid production are that petroleum is a nonrenewable starting material and that the oil refining process pollutes the environment.

To avoid petroleum-based production, researchers have proposed other methods for producing propionic acid. These involve fermentation of sugars by natural and engineered propionic acid producing microorganisms such as Propionibacterium acidipropionici using batch (Zhu, Y., et. al. Bioresource Technology (2010) 101: 8902-8906) and fed batch (Woskow, S. A. and Glatz. B. A. Appl. Environ. Microbiol. (1991) 57: 2821-2828) processes. However, productivities are very low because this bacterium is slow growing and can be product inhibited. Extractive (Solichien, M., et. al. Enzyme Microbial Technol. (1995) 17: 23-31) and cell-immobilized (Yang, S. T., et. al. Biotechnol. Bioeng. (1995) 45: 379-386) and cell recycle (Carrondo, M. J. T., et. al. Appl. Biochem. Biotech. (1987) 17: 295-312) fermentation processes have been developed to try to overcome these limitations, but all present issues limiting their application to large scale, inexpensive commercial production. Another approach is to engineer propionic acid production in faster growing industrial organisms such as Escherichia coli or Saccharomyces cerevisiae that can be grown using a fed batch processes.

Since at least 300,000 metric tons of propionic acid are produced annually, there remains a need in the art for cost-effective, environmentally-friendly methods for its production from renewable carbon sources.

### SUMMARY OF THE INVENTION

Most microorganisms do not make significant amounts of propionic acid, but microorganisms (such as bacteria, yeast, fungi or algae) are genetically modified according to the invention to carry out the conversions in the pathways. The present invention utilizes natural and artificial metabolic pathways to make propionate (the chemical form of propionic acid at neutral pH). FIGS. 1-3 set out the contemplated metabolic pathways for making propionic acid, which include either 2-ketobutyrate or succinyl-CoA as key intermediate metabolites. 2-Ketobutyrate may be synthesized by extension of the amino acid threonine biosynthetic pathway (FIG. 1) or the citramalate pathway (FIG. 2). Succinyl-CoA is an intermediate in the tricarboxylic acid pathway (TCA). The TCA may be split into reductive and oxidative branches (Bioengineered Bugs (2011) 2(2): 120-123) to produce succinyl-CoA in which propionic acid may be synthesized with a higher theoretical yield (1.7 mol/mol glucose, 70% g/g glucose) than other engineered pathways or the Wood-Werkman cycle utilized in Propionibacterium (54.8% g/g glucose) when the flux between oxidative branch and reductive branch is properly balanced. The oxidative branch produces additional reducing power for the reduction of pyruvate to priopionic acid without the production of acetic acid as the by product.

Producing Propionate

In a first embodiment, the invention provides a first type of microorganism, one that converts propionyl-CoA to propionate, wherein the microorganism expresses recombinant genes encoding a thioesterase, phosphate acyltransferase/kinase or acyl-CoA transferase.

The thioesterase, the phosphate acyltransferase/kinase or the acyl-CoA transferase catalyzes a reaction to convert propionyl-CoA to propionate. In some embodiments, the thioesterase is a propionyl-CoA thioesterase. Propionyl-CoA thioesterases include, but are not limited to Escherichia coli TesB set out in amino acid SEQ ID NO: 1, the Clostridium propionicum-derived thioesterase including an E324D substitution set out in SEQ ID NO: 3 and the Megasphaera elsdenii-derived thioesterase including an E325D substitution set out in SEQ ID NO: 5. Exemplary DNA sequences encoding these propionyl-CoA thioesterases are respectively set out in SEQ ID NOs: 2, 4 and 6. The amino acid sequence of a phosphate acyltransferase known in the art is set out in SEQ ID NO: 7. An exemplary DNA sequence encoding the phosphate acyltransferase is SEQ ID NO: 8. The amino acid sequence of a kinase known in the art is set out in SEQ ID NO: 9. An exemplary DNA sequence encoding the kinase is set out in SEQ ID NO: 10. The amino acid sequence of an acyl-CoA transferase known in the art is set out in SEQ ID NO: 11. An exemplary DNA sequence encoding the acyl-CoA transferase is set out in SEQ ID NO: 12.

In a second embodiment, the invention provides a second type of microorganism, one that converts threonine to propionate, wherein the microorganism expresses at least one recombinant gene selected from a group encoding: a dehydratase or deaminase, a dehydrogenase or lyase, and a thioesterase, phosphate acyltransferase/kinase or acyl-CoA transferase.

The dehydratase or deaminase catalyzes a reaction to convert threonine to 2-keto-butyrate. In some embodiments, the dehydratase is an L-amino acid dehydratase. Dehydratases include, but are not limited to, Klebsiella pneumoniae or Escherichia coli threonine dehydratase TdcB. The amino acid sequences of Klebsiella pneumoniae and Escherichia coli threonine dehydratase TdcB is known in the art and is set out in SEQ ID NOs: 13 and 15. Exemplary DNA sequences encoding Escherichia coli threonine dehydratase TdcB are set out in SEQ ID NOs: 14 and 16. In some embodiments, the deaminase is an L-amino acid deaminase. Deaminases include, but are not limited to, Escherichia coli threonine deaminase IlvA. The amino acid sequence of an Escherichia coli threonine deaminase IlvA is known in the art and is set out in SEQ ID NO: 17. An exemplary DNA sequence encoding Escherichia coli threonine deaminase IlvA is set out in SEQ ID NO: 18.

The dehydrogenase or combination of 2-ketoacid decarboxylase and Coenzyme-A acylating propionaldehyde dehydrogenase, or lyase catalyzes a reaction to convert 2-ketobutyrate to propionyl-CoA. In some embodiments, the dehydrogenase is a 2-ketoacid dehydrogenase. The 2-ketoacid dehydrogenases include, but are not limited to, pyruvate dehydrogenase PDH and branched chain keto acid dehydrogenase BKD. The pyruvate dehydrogenase is an enzyme complex containing 3 kinds of peptides set out in SEQ ID NOs: 19, 21 and 23. Exemplary DNA sequences encoding pyruvate dehydrogenase are set out in SEQ ID NOs: 20, 22 and 24. The branched chain keto acid dehydrogenase BKD set out in SEQ ID NOs: 25, 27, 29 and 31. Exemplary DNA sequences encoding branched chain keto acid dehydrogenase BKD are set out in SEQ ID NOs: 26, 28, 30 and 32. The 2-ketoacid decarboxylase KdcA is set out in SEQ ID NO: 33 and its derivatives. An exemplary DNA sequence encoding KdcA is set out in SEQ ID NO: 34. A Coenzyme-A acylating propionaldehyde dehydrogenase PduP is set out in SEQ ID NO: 35. An exemplary DNA sequence encoding PduP is set out in SEQ ID NO: 36 (codon optimized for Escherichia coli). In some embodiments, the lyase is a 2-ketoacid lyase. The 2-ketoacid lyases include, but are not limited to the 2-ketobutyrate formate lyase is set out in SEQ ID NO: 37 and its derivatives. An exemplary DNA sequence encoding 2-ketobutyrate formate lyase is set out in SEQ ID NO: 38.

The thioesterase, the phosphate acyltransferase/kinase or the acyl-CoA transferase catalyzes a reaction to convert propionyl-CoA to propionate. In some embodiments, the thioesterase is a propionyl-CoA thioesterase. Propionyl-CoA thioesterases include, but are not limited to Escherichia coli TesB set out in amino acid SEQ ID NO: 1, the Clostridium propionicum-derived thioesterase including an E324D substitution set out in SEQ ID NO: 3 and the Megasphaera elsdenii-derived thioesterase including an E325D substitution set out in SEQ ID NO: 5. Exemplary DNA sequences encoding these propionyl-CoA thioesterases are respectively set out in SEQ ID NOs: 2, 4 and 6. The amino acid sequence of a phosphate acyltransferase known in the art is set out in SEQ ID NO: 7. An exemplary DNA sequence encoding the phosphate acyltransferase is SEQ ID NO: 8. The amino acid sequence of a kinase known in the art is set out in SEQ ID NO: 9. An exemplary DNA sequence encoding the kinase is set out in SEQ ID NO: 10. The amino acid sequence of an acyl-CoA transferase known in the art is set out in SEQ ID NO: 11. An exemplary DNA sequence encoding the acyl-CoA transferase is set out in SEQ ID NO: 12.

In a third embodiment, the invention provides a first type of method, one for producing propionate in which the second type of microorganism is cultured to produce propionate. The second type of method for producing propionate converts threonine to 2-ketobutyrate, 2-ketobutyrate to propionyl-CoA, propionyl-CoA to propionate.

In a fourth embodiment, the invention provides a third type of microorganism, one that converts threonine to propionate, wherein the microorganism expresses recombinant genes selected from a group encoding: a dehydratase or deaminase, a 2-ketoacid decarboxylase, and an aldehyde dehydrogenase.

The dehydratase or deaminase catalyzes a reaction to convert threonine to 2-keto-butyrate. In some embodiments, the dehydratase is an L-amino acid dehydratase. Dehydratases include, but are not limited to, Klebsiella pneumoniae or Escherichia coli threonine dehydratase TdcB. The amino acid sequences of Klebsiella pneumoniae and Escherichia coli threonine dehydratase TdcB is known in the art and is set out in SEQ ID NOs: 13 and 15. Exemplary DNA sequences encoding Klebsiella pneumoniae and Escherichia coli threonine dehydratase TdcB are set out in SEQ ID NOs: 14 and 16. In some embodiments, the deaminase is an L-amino acid deaminase. Deaminases include, but are not limited to, Escherichia coli threonine deaminase IlvA. The amino acid sequence of an Escherichia coli threonine deaminase IlvA is known in the art and is set out in SEQ ID NO: 17. An exemplary DNA sequence encoding Escherichia coli threonine deaminase IlvA is set out in SEQ ID NO: 18.

The 2-ketoacid decarboxylase catalyzes a reaction to convert 2-ketobutyrate to propionaldehyde. The 2-ketoacid decarboxylase KdcA is set out in SEQ ID NO: 33 and its derivatives. An exemplary DNA sequence encoding KdcA is set out in SEQ ID NO: 34.

The aldehyde dehydrogenase catalyzes a reaction to convert propionaldehyde to propionic acid. Aldehyde dehydrogenases include, but are not limited to Pseudomonas sp. KIE171 aldehyde dehydrogenases IpuH and Ipul. Aldehyde dehydrogenases IpuH and Ipul are known in the art and are set out in SEQ ID NOs: 89 and 91. Exemplary DNA sequences encoding Pseudomonas sp. KIE171 aldehyde dehydrogenases IpuH and IupI are set out in SEQ ID NOs: 90 and 92.

In a fifth embodiment, the invention provides a second type of method, one for producing propionate in which the third type of microorganism is cultured to produce propionate. The third type of method for producing propionate converts threonine to 2-ketobutyrate, 2-ketobutyrate to propionaldehyde, and propionaldehyde to propionate.

In a sixth embodiment, the invention provides a fourth type of microorganism, one that converts succinyl-CoA to propionate, wherein the microorganism expresses recombinant genes selected from a group encoding: an acyl-CoA mutase, an acyl-CoA decarboxylase and a thioesterase, phosphate propionyltransferase/propionate kinase or acyl-CoA transferase.

The mutase catalyzes a reaction to convert succinyl-CoA to methylmalonyl-CoA. In some embodiments, the mutase is a methylmalonyl-CoA mutase. Mutases include, but are not limited to, methylmalonyl-CoA mutase. Amino acid sequences of the methylmalonyl-CoA mutase subunits A and B from Janibacter sp. HTCC2649 known in the art is set out in SEQ ID NOs: 39 and 41. Exemplary DNA sequences encoding the methylmalonyl-CoA mutase subunits A and B from Janibacter sp. HTCC2649 are respectively set out in SEQ ID NOs: 40 and 42.

The acyl-CoA decarboxylase catalyzes a reaction to convert methylmalonyl-CoA to propionyl-CoA. In some embodiments, the acyl-CoA decarboxylase is a methylmalonyl-CoA decarboxylase. The acyl-CoA decarboxylases include, but are not limited to, the Escherichia coli methylmalonyl-CoA decarboxylase YgfG set out in SEQ ID NO: 43 and its derivatives. An exemplary DNA sequence encoding the Escherichia coli methylmalonyl-CoA decarboxylase YgfG is set out in SEQ ID NO: 44.

The thioesterase, the phosphate acyltransferase/kinase or the acyl-CoA transferase catalyzes a reaction to convert propionyl-CoA to propionate. In some embodiments, the thioesterase is a propionyl-CoA thioesterase. Propionyl-CoA thioesterases include, but are not limited to Escherichia coli TesB set out in amino acid SEQ ID NO: 1, the Clostridium propionicum-derived thioesterase including an E324D substitution set out in SEQ ID NO: 3 and the Megasphaera elsdenii-derived thioesterase including an E325D substitution set out in SEQ ID NO: 5. Exemplary DNA sequences encoding these propionyl-CoA thioesterases are respectively set out in SEQ ID NOs: 2, 4 and 6. The amino acid sequence of a phosphate acyltransferase known in the art is set out in SEQ ID NO: 7. An exemplary DNA sequence encoding the phosphate acyltransferase is SEQ ID NO: 8. The amino acid sequence of a kinase known in the art is set out in SEQ ID NO: 9. An exemplary DNA sequence encoding the kinase is set out in SEQ ID NO: 10. The amino acid sequence of an acyl-CoA transferase known in the art is set out in SEQ ID NO: 11. An exemplary DNA sequence encoding the acyl-CoA transferase is set out in SEQ ID NO: 12.

In a seventh embodiment, the invention provides a third type of method, one for producing propionate in which the fourth type of microorganism is cultured to produce propionate. The third type of method for producing propionate converts succinyl-CoA to methylmalonyl-CoA, methylmalonyl-CoA to propionyl-CoA, and then propionyl-CoA to propionate.

In an eighth embodiment, the invention provides a fifth type of microorganism, one that converts pyruvate to propionate, wherein the microorganism expresses recombinant genes selected from a group encoding: a synthase, a hydrolase, a dehydratase or isomerase, a dehydrogenase and a ketoacid dehydrogenase or lyase and a thioesterase, phosphate propionyltransferase/propionate kinase or acyl-CoA transferase.

The synthase catalyzes a reaction to convert pyruvate to citramalate. In some embodiments, the synthase is a citramalate synthase. Synthases include, but are not limited to, citramalate synthase CimA from Methanobrevibacter ruminantium and Leptospira interrogans. Amino acid sequences of some synthases known in the art are set out in SEQ ID NOs: 45 and 47. Exemplary DNA sequences encoding those synthases are respectively set out in SEQ ID NOs: 46 and 48.

The hydrolase catalyzes a reaction to convert citramalate to citraconate. In some embodiments, the hydrolase is an isopropylmalate isomerase. Isomerases include, but are not limited to, isopropylmalate isomerase LeuC (large subunit) from Salmonella typhimurium. Amino acid sequences of an isopropylmalate isomerase LeuC from Salmonella typhimurium known in the art is set out in SEQ ID NO: 49. An exemplary DNA sequence encoding isopropylmalate isomerase (large subunit) LeuC from Salmonella typhimurium is respectively set out in SEQ ID NO: 50

The dehydratase, or isomerase, catalyzes a reaction to convert citraconate to β-methyl-D-malate. In some embodiments, the isomerase is an isopropylmalate isomerase. Amino acid sequences of an isopropylmalate isomerase (small subunit) LeuD from Salmonella typhimurium known in the art is set out in SEQ ID NO: 51. An exemplary DNA sequence encoding isopropylmalate isomerase LeuD from Salmonella typhimurium is respectively set out in SEQ ID NO: 52.

The dehydrogenase catalyzes a reaction to convert β-methyl-D-malate to 2-ketobutyrate. In some embodiments, dehydrogenase is a methylmalate dehydrogenase. In other embodiments, the dehydrogenase is a β-isopropylmalate dehydrogenase. Dehydrogenases include, but are not limited to, methylmalate dehydrogenase or Shigella boydii LeuB β-isopropylmalate dehydrogenase. The amino acid sequence of a LeuB β-isopropylmalate dehydrogenase is known in the art and set out in SEQ ID NO: 53. An exemplary DNA sequence encoding this LeuB β-isopropylmalate dehydrogenase is set out in SEQ ID NO: 54.

The dehydrogenase or combination of 2-ketoacid decarboxylase and Coenzyme-A acylating propionaldehyde dehydrogenase, or lyase catalyzes a reaction to convert 2-ketobutyrate to propionyl-CoA. In some embodiments, the dehydrogenase is a 2-ketoacid dehydrogenase. The 2-ketoacid dehydrogenases include, but are not limited to, pyruvate dehydrogenase PDH and branched chain keto acid dehydrogenase BKD. The pyruvate dehydrogenase is an enzyme complex containing 3 kinds of peptides set out in SEQ ID NOs: 19, 21 and 23. Exemplary DNA sequences encoding pyruvate dehydrogenase are set out in SEQ ID NOs: 20, 22 and 24. The branched chain keto acid dehydrogenase BKD set out in SEQ ID NOs: 25, 27, 29 and 31. Exemplary DNA sequences encoding branched chain keto acid dehydrogenase BKD are set out in SEQ ID NOs: 26, 28, 30 and 32. The 2-ketoacid decarboxylase KdcA is set out in SEQ ID NO: 33 and its derivatives. An exemplary DNA sequence encoding KdcA is set out in SEQ ID NO: 34. A Coenzyme-A acylating propionaldehyde dehydrogenase PduP is set out in SEQ ID NO: 35. An exemplary DNA sequence encoding PduP is set out in SEQ ID NO: 36 (codon optimized for Escherichia coli). In some embodiments, the lyase is a 2-ketoacid lyase. The 2-ketoacid lyases include, but are not limited to the 2-ketobutyrate formate lyase is set out in SEQ ID NO: 37 and its derivatives. An exemplary DNA sequence encoding 2-ketobutyrate formate lyase is set out in SEQ ID NO: 38.

The thioesterase, the phosphate acyltransferase/kinase or the acyl-CoA transferase catalyzes a reaction to convert propionyl-CoA to propionate. In some embodiments, the thioesterase is a propionyl-CoA thioesterase. Propionyl-CoA thioesterases include, but are not limited to Escherichia coli TesB set out in amino acid SEQ ID NO: 1, the Clostridium propionicum-derived thioesterase including an E324D substitution set out in SEQ ID NO: 3 and the Megasphaera elsdenii-derived thioesterase including an E325D substitution set out in SEQ ID NO: 5. Exemplary DNA sequences encoding these propionyl-CoA thioesterases are respectively set out in SEQ ID NOs: 2, 4 and 6. The amino acid sequence of a phosphate acyltransferase known in the art is set out in SEQ ID NO: 7. An exemplary DNA sequence encoding the phosphate acyltransferase is SEQ ID NO: 8. The amino acid sequence of a kinase known in the art is set out in SEQ ID NO: 9. An exemplary DNA sequence encoding the kinase is set out in SEQ ID NO: 10. The amino acid sequence of an acyl-CoA transferase known in the art is set out in SEQ ID NO: 11. An exemplary DNA sequence encoding the acyl-CoA transferase is set out in SEQ ID NO: 12.

In a ninth embodiment, the invention provides a fourth type of method, one for producing propionate in which the fifth type of microorganism is cultured to produce propionate. The fourth type of method for producing propionate converts pyruvate to citramalate, citramalate to citraconate, citraconate to β-methyl-D-malate, β-methyl-D-malate to 2-ketobutyrate, 2-ketobutyrate to propionyl-CoA, and propionyl-CoA to propionate.

In a tenth embodiment, the invention provides a sixth type of microorganism, one that converts pyruvate to propionate, wherein the microorganism expresses recombinant genes selected from a group encoding: a synthase, a hydrolase, a dehydratase or isomerase, a dehydrogenase, a 2-ketoacid decarboxylase and an aldehyde dehydrogenase.

The synthase catalyzes a reaction to convert pyruvate to citramalate. In some embodiments, the synthase is a citramalate synthase. Synthases include, but are not limited to, citramalate synthase cimA from Methanobrevibacter ruminantium and Leptospira interrogans. Amino acid sequences of some synthases known in the art are set out in SEQ ID NOs: 45 and 47. Exemplary DNA sequences encoding those synthases are respectively set out in SEQ ID NOs: 46 and 48.

The hydrolase catalyzes a reaction to convert citramalate to citraconate. In some embodiments, the hydrolase is an isopropylmalate isomerase. Isomerases include, but are not limited to, isopropylmalate isomerase LeuC (large subunit) from Salmonella typhimurium. Amino acid sequences of an isopropylmalate isomerase LeuC from Salmonella typhimurium known in the art is set out in SEQ ID NO: 49. An exemplary DNA sequence encoding isopropylmalate isomerase (large subunit) LeuC from Salmonella typhimurium is respectively set out in SEQ ID NO: 50.

The dehydratase, or isomerase, catalyzes a reaction to convert citraconate to β-methyl-D-malate. In some embodiments, the isomerase is an isopropylmalate isomerase. Amino acid sequences of an isopropylmalate isomerase (small subunit) LeuD from Salmonella typhimurium known in the art is set out in SEQ ID NO: 51. An exemplary DNA sequence encoding isopropylmalate isomerase LeuD from Salmonella typhimurium is respectively set out in SEQ ID NO: 52.

The dehydrogenase catalyzes a reaction to convert β-methyl-D-malate to 2-ketobutyrate. In some embodiments, dehydrogenase is a methylmalate dehydrogenase. In other embodiments, the dehydrogenase is a β-isopropylmalate dehydrogenase. Dehydrogenases include, but are not limited to, methylmalate dehydrogenase or Shigella boydii LeuB β-isopropylmalate dehydrogenase. The amino acid sequence of a LeuB β-isopropylmalate dehydrogenase is known in the art and set out in SEQ ID NO: 53. An exemplary DNA sequence encoding this LeuB (3-isopropylmalate dehydrogenase is set out in SEQ ID NO: 54.

The 2-ketoacid decarboxylase catalyzes a reaction to convert 2-ketobutyrate to propionaldehyde. The 2-ketoacid decarboxylase KdcA is set out in SEQ ID NO: 33 and its derivatives. An exemplary DNA sequence encoding KdcA is set out in SEQ ID NO: 34.

The aldehyde dehydrogenase catalyzes a reaction to convert propionaldehyde to propionic acid. Aldehyde dehydrogenases include, but are not limited to Pseudomonas sp. KIE171 aldehyde dehydrogenases IpuH and Ipul. Aldehyde dehydrogenases IpuH and Ipul are known in the art and are set out in SEQ ID NOs: 89 and 91. Exemplary DNA sequences encoding Pseudomonas sp. KIE171 aldehyde dehydrogenases IpuH and Ipul are set out in SEQ ID NOs: 90 and 92.

In an eleventh embodiment, the invention provides a seventh type of microorganism, one that converts pyruvate to propionate, wherein the microorganism expresses recombinant genes selected from a group encoding: an acyl-CoA transcarboxylase, a carboxylase, a dehydrogenase or a reductase, a dehydratase, a dehydrogenase, an acyl-CoA transferase or acyl-CoA synthetase, an acyl-CoA mutase, an acyl-CoA epimerase, an acyl-CoA decarboxylase and a thioesterase, phosphate propionyltransferase/propionate kinase or acyl-CoA transferase, a citrate synthase, an aconitase, an isocitrate dehydrogenase, and a dehydrogenase.

The acyl-CoA transcarboxylase or carboxylase catalyzes a reaction to convert pyruvate to oxaloacetate. In some embodiments, the transcarboxylase is a methylmalonyl-CoA transcarboxylase. Transcarboxylases include, but are not limited to, methylmalonyl-CoA transcarboxylase. Amino acid sequences of methylmalonyl-CoA transcarboxylases are known in the art are set out in SEQ ID NOs: 93, 95, 97, 99, 101, 103 and 105. Exemplary DNA sequences encoding methylmalonyl-CoA transcarboxylase are respectively set out in SEQ ID NOs: 94, 96, 98, 100, 102, 104 and 106. In some embodiments, the carboxylase is a pyruvate carboxylase. Carboxylases include, but are note limited to, pyruvate carboxylase. The amino acid sequence of a pyruvate carboxylase known in the art is set out in SEQ ID NO: 107. The exemplary DNA sequence encoding the pyruvate carboxylase known in the art is set out in SEQ ID NO: 108 The dehydrogenase catalyzes a reaction to convert oxaloacetate to malate. In some embodiments, the dehydrogenase is a malate dehydrogenase. Dehydrogenases include, but are not limited to, malate dehydrogenase. The amino acid sequence of malate dehydrogenase known in the art is set out in SEQ ID NOs: 109 and 111. The exemplary DNA sequences encoding malate dehydrogenase known in the art is set out in SEQ ID NOs: 110 and 112.

The dehydratase catalyzes a reaction to convert malate to fumarate. Dehydratases include, but are not limited to, fumarase. In some embodiments, the dehydratase is a fumarase. The amino acid sequence of fumases known in the art is set out in SEQ ID NOs: 113, 115, 117, 119 and 121. The exemplary DNA sequences encoding fumarases known in the art are set out in SEQ ID NOs: 114, 116, 118, 120 and 122.

The dehydrogenase or reductase catalyzes a reaction to convert fumarate to succinate. Dehydrogenases include, but are not limited to, succinate dehydrogenase. In some embodiments, the dehydrogenase is a succinate dehydrogenase. The amino acid sequence of succinate dehydrogenases known in the art are set out in SEQ ID NOs: 123, 125, 127, 129, 131, 133, 135, 137, 139 and 141. The exemplary DNA sequences encoding succinate dehydrogenase known in the art are set out in SEQ ID NOs: 124, 126, 128, 130, 132, 134, 136, 138, 140 and 142. Reductases include, but are not limited to, fumarate reductase. In some embodiments, the reductase is a fumurate reductase. The amino acid sequence of fumurate reductase known in the art is set out in SEQ ID NOs: 167, 169, 171 and 173. The exemplary DNA sequences encoding fumurate reductase known in the are are set out in SEQ ID NOs: 168, 170, 172 and 174.

The acyl-CoA transferase or acyl-CoA synthetase catalyzes a reaction to convert succinate to succinyl-CoA. Acyl-CoA transferases or acyl-CoA synthetases include, but are not limited to, succinyl-CoA transferase or succinyl-CoA synthetase. The amino acid sequence of an acyl-CoA transferase known in the art is set out in SEQ ID NO: 11. An exemplary DNA sequence encoding the acyl-CoA transferase is set out in SEQ ID NO: 12. The amino acid sequence of a succinyl-CoA synthetase known in the art is set out in SEQ ID NOs: 143 and 145. Exemplary DNA sequences encoding the succinyl-CoA synthetase known in the art are set out in SEQ ID NOs: 144 and 146.

The mutase catalyzes a reaction to convert succinyl-CoA to R-methylmalonyl-CoA. In some embodiments, the mutase is a methylmalonyl-CoA mutase. Mutases include, but are not limited to, methylmalonyl-CoA mutase. Amino acid sequences of the methylmalonyl-CoA mutase subunits A and B from Janibacter sp. HTCC2649 known in the art is set out in SEQ ID NOs: 39 and 41. Exemplary DNA sequences encoding the methylmalonyl-CoA mutase subunits A and B from Janibacter sp. HTCC2649 are respectively set out in SEQ ID NOs: 40 and 42.

The epimerase catalyzes a reaction to convert R-methylmalonyl-CoA to S-methylmalonyl-CoA.

In some embodiments, the mutase is a methylmalonyl-CoA epimerase. Epimerases include, but are not limited to, R-methylmalonyl-CoA epimerase. An amino acid sequence of a methylmalonyl-CoA epimerase known in the art is set out in SEQ ID NO: 175. An exemplary DNA sequence encoding the—methylmalonyl-CoA epimerase subunits A and B is set out in SEQ ID NO: 176.

The acyl-CoA decarboxylase catalyzes a reaction to convert S-methylmalonyl-CoA to propionyl-CoA. In some embodiments, the acyl-CoA decarboxylase is a methylmalonyl-CoA decarboxylase. The acyl-CoA decarboxylases include, but are not limited to, the Escherichia coli methylmalonyl-CoA decarboxylase YgfG set out in SEQ ID NO: 43 and its derivatives. An exemplary DNA sequence encoding the Escherichia coli methylmalonyl-CoA decarboxylase YgfG is set out in SEQ ID NO: 44.

The acyl-CoA transcarboxylase catalyzes a reaction to convert S-methylmalonyl-CoA to propionyl-CoA. In some embodiments, the transcarboxylase is a methylmalonyl-CoA transcarboxylase. Transcarboxylases include, but are not limited to, methylmalonyl-CoA transcarboxylase. Amino acid sequences of methylmalonyl-CoA transcarboxylases known in the art are set out in SEQ ID NOs: 93, 95, 97, 99, 101, 103 and 105. Exemplary DNA sequences encoding methylmalonyl-CoA transcarboxylases are respectively set out in SEQ ID NOs: 94, 96, 98, 100, 102, 104 and 106.

The thioesterase, the phosphate acyltransferase/kinase or the acyl-CoA transferase catalyzes a reaction to convert propionyl-CoA to propionate. In some embodiments, the thioesterase is a propionyl-CoA thioesterase. Propionyl-CoA thioesterases include, but are not limited to Escherichia coli TesB set out in amino acid SEQ ID NO: 1, the Clostridium propionicum-derived thioesterase including an E324D substitution set out in SEQ ID NO: 3 and the Megasphaera elsdenii-derived thioesterase including an E325D substitution set out in SEQ ID NO: 5. Exemplary DNA sequences encoding these propionyl-CoA thioesterases are respectively set out in SEQ ID NOs: 2, 4 and 6. The amino acid sequence of a phosphate acyltransferase known in the art is set out in SEQ ID NO: 7. An exemplary DNA sequence encoding the phosphate acyltransferase is SEQ ID NO: 8. The amino acid sequence of a kinase known in the art is set out in SEQ ID NO: 9. An exemplary DNA sequence encoding the kinase is set out in SEQ ID NO: 10. The amino acid sequence of an acyl-CoA transferase known in the art is set out in SEQ ID NO: 11. An exemplary DNA sequence encoding the acyl-CoA transferase is set out in SEQ ID NO: 12.

The synthase catalyzes a reaction to convert oxaloacetate and acetyl-CoA to citrate. In some embodiments, the synthase is a citrate synthase. An amino acid sequence of a citrate synthase known in the art is set out in SEQ ID NO: 153. An exemplary DNA sequence encoding the citrate synthase is respectively set out in SEQ ID NO: 154.

The aconitase catalyzes a reaction to convert citrate to aconitate, and aconitate to isocitrate. Amino acid sequences of aconitase known in the art are set out in SEQ ID NOs: 155 and 157. Exemplary DNA sequences encoding aconitase are respectively set out in SEQ ID NOs: 156 and 158.

The dehydrogenase catalyzes a reaction to convert isocitrate to α-ketoglutarate. In some embodiments, the dehydrogenase is an isocitrate dehydrogenase. The amino acid sequence of an isocitrate dehydrogenase known in the art is set out in SEQ ID NO: 159. The exemplary DNA sequence encoding isocitrate dehydrogenase known in the art is set out in SEQ ID NO: 160.

The dehydrogenase catalyzes a reaction to convert α-ketoglutarate to succinyl-CoA. In some embodiments, the dehydrogenase is an α-ketoglutarate dehydrogenase. Dehydrogenases include, but are not limited to, α-ketoglutarate dehydrogenase. The amino acid sequences of α-ketoglutarate dehydrogenase known in the art are set out in SEQ ID NOs: 161, 163 and 165. The exemplary DNA sequences encoding α-ketoglutarate dehydrogenase known in the art are set out in SEQ ID NOs: 162, 164 and 166.

In a twelfth embodiment, the invention provides a fifth type of method, one for producing propionate in which the seventh type of microorganism is cultured to produce propionate. The fifth type of method for producing propionate converts pyruvate to oxaloacetate, oxaloacetate to malate and citrate, malate to fumarate, fumarate to succinate, succinate to succinyl-CoA, and citrate to isocitrate, isocitrate to α-ketoglutarate, α-ketoglutarate to succinyl-CoA, succinyl-CoA to methylmalonyl-CoA, methylmalonyl-CoA to propionyl-CoA and propionyl-CoA to propionate.

In a thirteenth embodiment, the invention provides an eighth type of microorganism, one that converts phosphoenolpyruvate to propionate, wherein the microorganism expresses recombinant genes selected from a group encoding: a carboxykinase or a carboxylase, a dehydrogenase, a dehydratase, a dehydrogenase or reductase, an acyl-CoA transferase or acyl-CoA synthetase, an acyl-CoA mutase, an acyl-CoA epimerase, an acyl-CoA decarboxylase, a thioesterase, phosphate propionyltransferase/propionate kinase or acyl-CoA transferase, a citrate synthase, an aconitase, an isocitrate dehydrogenase, a dehydrogenase.

The carboxykinase or carboxylase catalyzes a reaction to convert phosphoenolpyruvate to oxaloacetate. In some embodiments, the carboxykinase is a phosphoenolpyruvate carboxykinase. Carboxykinases include, but are not limited to, phosphoenolpyruvate carboxykinase. The amino acid sequence of a phosphoenolpyruvate carboxykinase known in the art is set out in SEQ ID NO: 147. The exemplary DNA sequence encoding the phosphoenolpyruvate carboxykinase known in the art is set out in SEQ ID NO: 148. In some embodiments, the carboxylase is a phosphoenolpyruvate carboxylase. Carboxylases include, but are not limited to, phosphoenolpyruvate carboxylase. The amino acid sequence of a phosphoenolpyruvate carboxylase known in the art is set out in SEQ ID NO: 55. The exemplary DNA sequence encoding the phosphoenolpyruvate carboxylase known in the art is set out in SEQ ID NO: 56.

The dehydrogenase catalyzes a reaction to convert oxaloacetate to malate. In some embodiments, the dehydrogenase is a malate dehydrogenase. Dehydrogenases include, but are not limited to, malate dehydrogenase. The amino acid sequences of malate dehydrogenase known in the art are set out in SEQ ID NOs: 109 and 111. The exemplary DNA sequences encoding malate dehydrogenase known in the art are set out in SEQ ID NOs: 110 and 112.

The dehydratase catalyzes a reaction to convert malate to fumarate. Dehydratases include, but are not limited to, fumarase. In some embodiments, the dehydratase is a fumarase. The amino acid sequences of fumases known in the art are set out in SEQ ID NOs: 113, 115, 117, 119 and 121. The exemplary DNA sequences encoding fumarases known in the art are set out in SEQ ID NOs: 114, 116, 118, 120 and 122.

The dehydrogenase or reductase catalyzes a reaction to convert fumarate to succinate. Dehydrogenases include, but are not limited to, succinate dehydrogenase. In some embodiments, the dehydrogenase is a succinate dehydrogenase. The amino acid sequences of succinate dehydrogenases known in the art are set out in SEQ ID NOs: 123, 125, 127, 129, 131, 133, 135, 137, 139 and 141. The exemplary DNA sequences encoding succinate dehydrogenase known in the are are set out in SEQ ID NOs: 124, 126, 128, 130, 132, 134, 136, 138, 140 and 142. Reductases include, but are not limited to, fumarate reductase. In some embodiments, the reductase is a fumurate reductase. The amino acid sequences of fumurate reductase known in the art are set out in SEQ ID NOs: 167, 169, 171 and 173. The exemplary DNA sequences encoding fumurate reductase known in the are are set out in SEQ ID NOs: 168, 170, 172, and 174.

The acyl-CoA transferase or acyl-CoA synthetase catalyzes a reaction to convert succinate to succinyl-CoA. Acyl-CoA transferases or acyl-CoA synthetases include, but are not limited to, succinyl-CoA transferase or succinyl-CoA synthetase. The amino acid sequence of an acyl-CoA transferase known in the art is set out in SEQ ID NO: 11. An exemplary DNA sequence encoding the acyl-CoA transferase is set out in SEQ ID NO: 12. The amino acid sequences of a succinyl-CoA synthetase known in the art are set out in SEQ ID NOs: 143 and 145. Exemplary DNA sequences encoding the succinyl-CoA synthetase known in the art are set out in SEQ ID NOs: 144 and 146.

The mutase catalyzes a reaction to convert succinyl-CoA to methylmalonyl-CoA. In some embodiments, the mutase is a methylmalonyl-CoA mutase. Mutases include, but are not limited to, methylmalonyl-CoA mutase. Amino acid sequences of the methylmalonyl-CoA mutase subunits A and B from Janibacter sp. HTCC2649 known in the art are set out in SEQ ID NOs: 39 and 41. Exemplary DNA sequences encoding the methylmalonyl-CoA mutase subunits A and B from Janibacter sp. HTCC2649 are respectively set out in SEQ ID NOs: 40 and 42.

The epimerase catalyzes a reaction to convert R-methylmalonyl-CoA to S-methylmalonyl-CoA. In some embodiments, the mutase is a methylmalonyl-CoA epimerase. Epimerases include, but are not limited to, R-methylmalonyl-CoA epimerase. The amino acid sequence of a methylmalonyl-CoA epimerase known in the art is set out in SEQ ID NO: 175. An exemplary DNA sequence encoding the—methylmalonyl-CoA epimerase is set out in SEQ ID NO: 176.

The acyl-CoA decarboxylase catalyzes a reaction to convert methylmalonyl-CoA to propionyl-CoA. In some embodiments, the acyl-CoA decarboxylase is a methylmalonyl-CoA decarboxylase. The acyl-CoA decarboxylases include, but are not limited to, the Escherichia coli methylmalonyl-CoA decarboxylase YgfG set out in SEQ ID NO: 43 and its derivatives. An exemplary DNA sequence encoding the Escherichia coli methylmalonyl-CoA decarboxylase YgfG is set out in SEQ ID NO: 44. The acyl-CoA transcarboxylase catalyzes a reaction to convert methylmalonyl-CoA to propionyl-CoA. In some embodiments, the transcarboxylase is a methylmalonyl-CoA transcarboxylase. Transcarboxylases include, but are not limited to, methylmalonyl-CoA transcarboxylase. Amino acid sequences of methylmalonyl-CoA transcarboxylases known in the art are set out in SEQ ID NOs: 93, 95, 97, 99, 101, 103 and 105. Exemplary DNA sequences encoding methylmalonyl-CoA transcarboxylases are respectively set out in SEQ ID NOs: 94, 96, 98, 100, 102, 104 and 106.

The thioesterase, the phosphate acyltransferase/kinase or the acyl-CoA transferase catalyzes a reaction to convert propionyl-CoA to propionate. In some embodiments, the thioesterase is a propionyl-CoA thioesterase. Propionyl-CoA thioesterases include, but are not limited to Escherichia coli TesB set out in amino acid SEQ ID NO: 1, the Clostridium propionicum-derived thioesterase including an E324D substitution set out in SEQ ID NO: 3 and the Megasphaera elsdenii-derived thioesterase including an E325D substitution set out in SEQ ID NO: 5. Exemplary DNA sequences encoding these propionyl-CoA thioesterases are respectively set out in SEQ ID NOs: 2, 4 and 6. The amino acid sequence of a phosphate acyltransferase known in the art is set out in SEQ ID NO: 7. An exemplary DNA sequence encoding the phosphate acyltransferase is SEQ ID NO: 8. The amino acid sequence of a kinase known in the art is set out in SEQ ID NO: 9. An exemplary DNA sequence encoding the kinase is set out in SEQ ID NO: 10. The amino acid sequence of an acyl-CoA transferase known in the art is set out in SEQ ID NO: 11. An exemplary DNA sequence encoding the acyl-CoA transferase is set out in SEQ ID NO: 12.

The synthase catalyzes a reaction to convert oxaloacetate and acetyl-CoA to citrate. In some embodiments, the synthase is a citrate synthase. An amino acid sequence of a citrate synthase known in the art is set out in SEQ ID NO: 153. An exemplary DNA sequence encoding the citrate synthase is respectively set out in SEQ ID NO: 154.

The aconitase catalyzes a reaction to convert citrate to aconitate, and aconitate to isocitrate. Amino acid sequences of aconitase known in the art are set out in SEQ ID NOs: 155 and 157. Exemplary DNA sequences encoding the aconitases are respectively set out in SEQ ID NOs: 156 and 158.

The dehydrogenase catalyzes a reaction to convert isocitrate to α-ketoglutarate. In some embodiments, the dehydrogenase is an isocitrate dehydrogenase. The amino acid sequence of an isocitrate dehydrogenase known in the art is set out in SEQ ID NO: 159. The exemplary DNA sequence encoding isocitrate dehydrogenase known in the art is set out in SEQ ID NO: 160.

The dehydrogenase catalyzes a reaction to convert α-ketoglutarate to succinyl-CoA. In some embodiments, the dehydrogenase is an α-ketoglutarate dehydrogenase. Dehydrogenases include, but are not limited to, α-ketoglutarate dehydrogenase. The amino acid sequences of α-ketoglutarate dehydrogenase known in the art are set out in SEQ ID NOs: 161, 163 and 165. The exemplary DNA sequences encoding α-ketoglutarate dehydrogenase known in the art are set out in SEQ ID NOs: 162, 164 and 166

In an fourteenth embodiment, the invention provides a sixth type of method, one for producing propionate in which the eighth type of microorganism is cultured to produce propionate. The sixth type of method for producing propionate converts phsophoenolpyruvate to oxaloacetate, oxaloacetate to malate and citrate, malate to fumarate, fumarate to succinate, succinate to succinyl-CoA, citrate to isocitrate, isocitrate to α-ketoglutarate, α-ketoglutarate to succinyl-CoA, succinyl-CoA to methylmalonyl-CoA, methylmalonyl-CoA to propionyl-CoA and propionyl-CoA to propionate.

Use of Isolated Enzymes

In a fifteenth embodiment, the invention provides for a seventh method using isolated enzymes or from a cell lysate, one that converts threonine to propionate, wherein the enzymes include at least one selected from a group comprising a dehydratase, a dehydrogenase or lyase, and a thioesterase, phosphate acyltransferase/kinase or acyl-CoA transferase.

The dehydratase or deaminase catalyzes a reaction to convert threonine to 2-keto-butyrate. In some embodiments, the dehydratase is an L-amino acid dehydratase. Dehydratases include, but are not limited to, Klebsiella pneumoniae or Escherichia coli threonine dehydratase TdcB. The amino acid sequences of Klebsiella pneumoniae and Escherichia coli threonine dehydratase TdcB are known in the art and are set out in SEQ ID NOs: 13 and 15. Exemplary DNA sequences encoding Klebsiella pneumoniae and Escherichia coli threonine dehydratase TdcB are set out in SEQ ID NOs: 14 and 16. In some embodiments, the deaminase is an L-amino acid deaminase. Deaminases include, but are not limited to, Escherichia coli threonine deaminase IlvA. The amino acid sequence of an Escherichia coli threonine deaminase IlvA is known in the art and is set out in SEQ ID NO: 21. An exemplary DNA sequence encoding Escherichia coli threonine deaminase IlvA is set out in SEQ ID NO: 22.

The dehydrogenase or combination of 2-ketoacid decarboxylase and Coenzyme-A acylating propionaldehyde dehydrogenase, or lyase catalyzes a reaction to convert 2-ketobutyrate to propionyl-CoA. In some embodiments, the dehydrogenase is a 2-ketoacid dehydrogenase. The 2-ketoacid dehydrogenases include, but are not limited to, pyruvate dehydrogenase PDH and branched chain keto acid dehydrogenase BKD. The pyruvate dehydrogenase is an enzyme complex containing 3 kinds of peptides set out in SEQ ID NOs: 19, 21 and 23. Exemplary DNA sequences encoding pyruvate dehydrogenase are set out in SEQ ID NOs: 20, 22 and 24. The branched chain keto acid dehydrogenase BKD is set out in SEQ ID NOs: 25, 27, 29 and 31. Exemplary DNA sequences encoding branched chain keto acid dehydrogenase BKD are set out in SEQ ID NOs: 26, 28, 30 and 32. The 2-ketoacid decarboxylase KdcA is set out in SEQ ID NO: 33 and its derivatives. An exemplary DNA sequence encoding KdcA is set out in SEQ ID NO: 34. A Coenzyme-A acylating propionaldehyde dehydrogenase PduP is set out in SEQ ID NO: 35. An exemplary DNA sequence encoding PduP is set out in SEQ ID NO: 36 (codon optimized for Escherichia coli). In some embodiments, the lyase is a 2-ketoacid lyase. The 2-ketoacid lyases include, but are not limited to the 2-ketobutyrate formate lyase is set out in SEQ ID NO: 37 and its derivatives. An exemplary DNA sequence encoding 2-ketobutyrate formate lyase is set out in SEQ ID NO: 38.

The thioesterase, the phosphate acyltransferase/kinase or the acyl-CoA transferase catalyzes a reaction to convert propionyl-CoA to propionate. In some embodiments, the thioesterase is a propionyl-CoA thioesterase. Propionyl-CoA thioesterases include, but are not limited to Escherichia coli TesB set out in amino acid SEQ ID NO: 1, the Clostridium propionicum-derived thioesterase including an E324D substitution set out in SEQ ID NO: 3 and the Megasphaera elsdenii-derived thioesterase including an E325D substitution set out in SEQ ID NO: 5. Exemplary DNA sequences encoding these propionyl-CoA thioesterases are respectively set out in SEQ ID NOs: 2, 4 and 6. The amino acid sequence of a phosphate acyltransferase known in the art is set out in SEQ ID NO: 7. An exemplary DNA sequence encoding the phosphate acyltransferase is SEQ ID NO: 8. The amino acid sequence of a kinase known in the art is set out in SEQ ID NO: 9. An exemplary DNA sequence encoding the kinase is set out in SEQ ID NO: 10. The amino acid sequence of an acyl-CoA transferase known in the art is set out in SEQ ID NO: 11. An exemplary DNA sequence encoding the acyl-CoA transferase is set out in SEQ ID NO: 12.

In an sixteenth embodiment, the invention provides for a eighth method using isolated enzymes or from a cell lysate, one that converts threonine to proprionate, wherein the enzymes can include at least one selected from a group comprising a dehydratase or deaminase, a 2-ketoacid decarboxylase, and an aldehyde dehydrogenase.

The dehydratase or deaminase catalyzes a reaction to convert threonine to 2-keto-butyrate. In some embodiments, the dehydratase is an L-amino acid dehydratase. Dehydratases include, but are not limited to, Klebsiella pneumoniae or Escherichia coli threonine dehydratase TdcB. The amino acid sequences of Klebsiella pneumoniae and Escherichia coli threonine dehydratase TdcB are known in the art and are set out in SEQ ID NOs: 13 and 15. Exemplary DNA sequences encoding Klebsiella pneumoniae and Escherichia coli threonine dehydratase TdcB are set out in SEQ ID NOs: 14 and 16. In some embodiments, the deaminase is an L-amino acid deaminase. Deaminases include, but are not limited to, Escherichia coli threonine deaminase IlvA. The amino acid sequence of an Escherichia coli threonine deaminase IlvA is known in the art and is set out in SEQ ID NO: 21. An exemplary DNA sequence encoding Escherichia coli threonine deaminase IlvA is set out in SEQ ID NO: 22.

The 2-ketoacid decarboxylase catalyzes a reaction to convert 2-ketobutyrate to propionaldehyde. The 2-ketoacid decarboxylase KdcA is set out in SEQ ID NO: 33 and its derivatives. An exemplary DNA sequence encoding KdcA is set out in SEQ ID NO: 34.

The aldehyde dehydrogenase catalyzes a reaction to convert propionaldehyde to propionic acid. Aldehyde dehydrogenases include, but are not limited to Pseudomonas sp. KIE171 aldehyde dehydrogenases IpuH and Ipul. Aldehyde dehydrogenases IpuH and Ipul are known in the art and are set out in SEQ ID NOs: 89 and 91. Exemplary DNA sequences encoding Pseudomonas sp. KIE171 aldehyde dehydrogenases IpuH and Ipul are set out in SEQ ID NOs: 90 and 92.

In a seventeenth embodiment, the invention provides for a ninth method using isolated purified enzymes or from a cell lysate, one that converts succinate to propionate, wherein the enzymes can include at least one selected from a group comprising an acyl-CoA mutase, an acyl-CoA decarboxylase, and a thioesterase, phosphate acyltransferase/kinase or acyl-CoA transferase.

The mutase catalyzes a reaction to convert succinyl-CoA to methylmalonyl-CoA. In some embodiments, the mutase is a methylmalonyl-CoA mutase. Mutases include, but are not limited to, methylmalonyl-CoA mutase. Amino acid sequences of the methylmalonyl-CoA mutase subunits A and B from Janibacter sp. HTCC2649 known in the art are set out in SEQ ID NOs: 39 and 41. Exemplary DNA sequences encoding the methylmalonyl-CoA mutase subunits A and B from Janibacter sp. HTCC2649 are respectively set out in SEQ ID NOs: 40 and 42.

The acyl-CoA decarboxylase catalyzes a reaction to convert methylmalonyl-CoA to propionyl-CoA. In some embodiments, the acyl-CoA decarboxylase is a methylmalonyl-CoA decarboxylase. The acyl-CoA decarboxylases include, but are not limited to, the Escherichia coli methylmalonyl-CoA decarboxylase YgfG set out in SEQ ID NO: 43 and its derivatives. An exemplary DNA sequence encoding the Escherichia coli methylmalonyl-CoA decarboxylase YgfG is set out in SEQ ID NO: 44.

The thioesterase, the phosphate acyltransferase/kinase or the acyl-CoA transferase catalyzes a reaction to convert propionyl-CoA to propionate. In some embodiments, the thioesterase is a propionyl-CoA thioesterase. Propionyl-CoA thioesterases include, but are not limited to Escherichia coli TesB set out in amino acid SEQ ID NO: 1, the Clostridium propionicum-derived thioesterase including an E324D substitution set out in SEQ ID NO: 3 and the Megasphaera elsdenii-derived thioesterase including an E325D substitution set out in SEQ ID NO: 5. Exemplary DNA sequences encoding these propionyl-CoA thioesterases are respectively set out in SEQ ID NOs: 2, 4 and 6. The amino acid sequence of a phosphate acyltransferase known in the art is set out in SEQ ID NO: 7. An exemplary DNA sequence encoding the phosphate acyltransferase is SEQ ID NO: 8. The amino acid sequence of a kinase known in the art is set out in SEQ ID NO: 9. An exemplary DNA sequence encoding the kinase is set out in SEQ ID NO: 10. The amino acid sequence of an acyl-CoA transferase known in the art is set out in SEQ ID NO: 11. An exemplary DNA sequence encoding the acyl-CoA transferase is set out in SEQ ID NO: 12.

In an eighteenth embodiment, the invention provides for a tenth method using isolated enzymes or from a cell lysate, one that converts pyruvate, citramalate, citraconate, β-methyl-D-malate or 2-ketobutyrate to propionate, wherein the enzymes can include at least one selected from a group comprising a synthase, a hydrolase, a dehydratase or isomerase, a dehydrogenase and a ketoacid dehydrogenase, and a thioesterase, phosphate transferase/kinase or acyl-CoA transferase.

The synthase catalyzes a reaction to convert pyruvate to citramalate. In some embodiments, the synthase is a citramalate synthase. Synthases include, but are not limited to, citramalate synthase CimA from Methanobrevibacter ruminantium and Leptospira interrogans. Amino acid sequences of some synthases known in the art are set out in SEQ ID NOs: 45 and 47. Exemplary DNA sequences encoding those synthases are respectively set out in SEQ ID NOs: 46 and 48.

The hydrolase catalyzes a reaction to convert citramalate to citraconate. In some embodiments, the hydrolase is an isopropylmalate isomerase. Isomerases include, but are not limited to, isopropylmalate isomerase LeuC (large subunit) from Salmonella typhimurium. Amino acid sequences of an isopropylmalate isomerase LeuC from Salmonella typhimurium known in the art is set out in SEQ ID NO: 49. An exemplary DNA sequence encoding isopropylmalate isomerase (large subunit) LeuC from Salmonella typhimurium is respectively set out in SEQ ID NO: 50

The dehydratase, or isomerase, catalyzes a reaction to convert citraconate to β-methyl-D-malate. In some embodiments, the isomerase is an isopropylmalate isomerase. Amino acid sequences of an isopropylmalate isomerase (small subunit) LeuD from Salmonella typhimurium known in the art is set out in SEQ ID NO: 51. An exemplary DNA sequence encoding isopropylmalate isomerase LeuD from Salmonella typhimurium is respectively set out in SEQ ID NO: 52.

The dehydrogenase catalyzes a reaction to convert β-methyl-D-malate to 2-ketobutyrate. In some embodiments, dehydrogenase is a methylmalate dehydrogenase. In other embodiments, the dehydrogenase is a β-isopropylmalate dehydrogenase. Dehydrogenases include, but are not limited to, methylmalate dehydrogenase or Shigella boydii LeuB β-isopropylmalate dehydrogenase. The amino acid sequence of a LeuB β-isopropylmalate dehydrogenase is known in the art and set out in SEQ ID NO: 53. An exemplary DNA sequence encoding this LeuB β-isopropylmalate dehydrogenase is set out in SEQ ID NO: 54.

The dehydrogenase or combination of 2-ketoacid decarboxylase and Coenzyme-A acylating propionaldehyde dehydrogenase, or lyase catalyzes a reaction to convert 2-ketobutyrate to propionyl-CoA. In some embodiments, the dehydrogenase is a 2-ketoacid dehydrogenase. The 2-ketoacid dehydrogenases include, but are not limited to, pyruvate dehydrogenase PDH and branched chain keto acid dehydrogenase BKD. The pyruvate dehydrogenase is an enzyme complex containing 3 kinds of peptides set out in SEQ ID NOs: 19, 21 and 23. Exemplary DNA sequences encoding pyruvate dehydrogenase are set out in SEQ ID NOs: 20, 22 and 24. The branched chain keto acid dehydrogenase BKD is set out in SEQ ID NOs: 25, 27, 29 and 31. Exemplary DNA sequences encoding branched chain keto acid dehydrogenase BKD are set out in SEQ ID NOs: 26, 28, 30 and 32. The 2-ketoacid decarboxylase KdcA is set out in SEQ ID NO: 33 and its derivatives. An exemplary DNA sequence encoding KdcA is set out in SEQ ID NO: 34. A Coenzyme-A acylating propionaldehyde dehydrogenase PduP is set out in SEQ ID NO: 35. An exemplary DNA sequence encoding PduP is set out in SEQ ID NO: 36 (codon optimized for Escherichia coli). In some embodiments, the lyase is a 2-ketoacid lyase. The 2-ketoacid lyases include, but are not limited to the 2-ketobutyrate formate lyase is set out in SEQ ID NO: 37 and its derivatives. An exemplary DNA sequence encoding 2-ketobutyrate formate lyase is set out in SEQ ID NO: 38.

The thioesterase, the phosphate acyltransferase/kinase or the acyl-CoA transferase catalyzes a reaction to convert propionyl-CoA to propionate. In some embodiments, the thioesterase is a propionyl-CoA thioesterase. Propionyl-CoA thioesterases include, but are not limited to Escherichia coli TesB set out in amino acid SEQ ID NO: 1, the Clostridium propionicum-derived thioesterase including an E324D substitution set out in SEQ ID NO: 3 and the Megasphaera elsdenii-derived thioesterase including an E325D substitution set out in SEQ ID NO: 5. Exemplary DNA sequences encoding these propionyl-CoA thioesterases are respectively set out in SEQ ID NOs: 2, 4 and 6. The amino acid sequence of a phosphate acyltransferase known in the art is set out in SEQ ID NO: 7. An exemplary DNA sequence encoding the phosphate acyltransferase is SEQ ID NO: 8. The amino acid sequence of a kinase known in the art is set out in SEQ ID NO: 9. An exemplary DNA sequence encoding the kinase is set out in SEQ ID NO: 10. The amino acid sequence of an acyl-CoA transferase known in the art is set out in SEQ ID NO: 11. An exemplary DNA sequence encoding the acyl-CoA transferase is set out in SEQ ID NO: 12.

In an nineteenth embodiment, the invention provides for a eleventh method using isolated purified enzymes or from a cell lysate, one that converts pyruvate, citramalate, citraconate, β-methyl-D-malate or 2-ketobutyrate to propionate, wherein the enzymes can include at least one selected from a group comprising a synthase, a hydrolase, a dehydratase or isomerase, a 2-ketoacid decarboxylase, and an aldehyde dehydrogenase.

The synthase catalyzes a reaction to convert pyruvate to citramalate. In some embodiments, the synthase is a citramalate synthase. Synthases include, but are not limited to, citramalate synthase CimA from Methanobrevibacter ruminantium and Leptospira interrogans. Amino acid sequences of some synthases known in the art are set out in SEQ ID NOs: 45 and 47. Exemplary DNA sequences encoding those synthases are respectively set out in SEQ ID NOs: 46 and 48.

The hydrolase catalyzes a reaction to convert citramalate to citraconate. In some embodiments, the hydrolase is an isopropylmalate isomerase. Isomerases include, but are not limited to, isopropylmalate isomerase LeuC (large subunit) from Salmonella typhimurium. Amino acid sequences of an isopropylmalate isomerase LeuC from Salmonella typhimurium known in the art is set out in SEQ ID NO: 49. An exemplary DNA sequence encoding isopropylmalate isomerase (large subunit) LeuC from Salmonella typhimurium is respectively set out in SEQ ID NO: 50

The dehydratase, or isomerase, catalyzes a reaction to convert citraconate to β-methyl-D-malate. In some embodiments, the isomerase is an isopropylmalate isomerase. Amino acid sequences of an isopropylmalate isomerase (small subunit) LeuD from Salmonella typhimurium known in the art is set out in SEQ ID NO: 51. An exemplary DNA sequence encoding isopropylmalate isomerase LeuD from Salmonella typhimurium is respectively set out in SEQ ID NO: 52.

The dehydrogenase catalyzes a reaction to convert β-methyl-D-malate to 2-ketobutyrate. In some embodiments, dehydrogenase is a methylmalate dehydrogenase. In other embodiments, the dehydrogenase is a β-isopropylmalate dehydrogenase. Dehydrogenases include, but are not limited to, methylmalate dehydrogenase or Shigella boydii LeuB β-isopropylmalate dehydrogenase. The amino acid sequence of a LeuB β-isopropylmalate dehydrogenase is known in the art and set out in SEQ ID NO: 53. An exemplary DNA

The 2-ketoacid decarboxylase catalyzes a reaction to convert 2-ketobutyrate to propionaldehyde. The 2-ketoacid decarboxylase KdcA is set out in SEQ ID NO: 33 and its derivatives. An exemplary DNA sequence encoding KdcA is set out in SEQ ID NO: 34.

The aldehyde dehydrogenase catalyzes a reaction to convert propionaldehyde to propionic acid. Aldehyde dehydrogenases include, but are not limited to Pseudomonas sp. KIE171 aldehyde dehydrogenases ipuH and ipul. Aldehyde dehydrogenases IpuH and Ipul are known in the art and are set out in SEQ ID NOs: 89 and 91. Exemplary DNA sequences encoding Pseudomonas sp. KIE171 aldehyde dehydrogenases IpuH and Ipulare set out in SEQ ID NOs: 90 and 92.

Increasing the Carbon Flow to Threonine

In a twentieth embodiment, the invention provides microorganisms that include further genetic modifications in order to increase the carbon flow to threonine which, in turn, increases the production of propionate. The microorganisms exhibit one or more of the following characteristics.

In some embodiments, the microorganism exhibits increased carbon flow to oxaloacetate in comparison to a corresponding wild-type microorganism. The microorganism expresses a recombinant gene encoding, for example, phosphoenolpyruvate carboxylase or pyruvate carboxylase (or both). The phosphoenolpyruvate carboxylases include, but are not limited to, the phosphoenolpyruvate carboxylase set out in SEQ ID NO: 55. An exemplary DNA sequence encoding the phosphoenolpyruvate carboxylase is set out in SEQ ID NO: 56. The pyruvate carboxylases include, but are not limited to, the pyruvate carboxylases set out in SEQ ID NOs: 57 and 59. Exemplary DNA sequences encoding the pyruvate carboxylases are set out in SEQ ID NOS: 58 and 60.

In some embodiments, the microorganism exhibits reduced aspartate kinase feedback inhibition in comparison to a corresponding wild-type microorganism. The microorganism expresses one or more of the genes encoding the polypeptides including, but not limited to, S345F ThrA (SEQ ID NO: 61), T352I LysC (SEQ ID NO: 63) and MetL (SEQ ID NO: 65). Exemplary coding sequences encoding the polypeptides are respectively set out in SEQ ID NO: 62, SEQ ID NO: 64 and SEQ ID NO: 66.

In some embodiments, the microorganism exhibits reduced lysA gene expression or diaminopimelate decarboxylase activity in comparison to a corresponding wild-type microorganism. In some embodiments, the microorganism exhibits reduced dapA expression or dihydropicolinate synthase activity in comparison to a corresponding wild type organism. An exemplary DNA sequence of a lysA coding sequence known in the art is set out in SEQ ID NO: 68. It encodes the amino acid sequence set out in SEQ ID NO: 67. An exemplary DNA sequence of a dapA coding sequence known in the art is set out in SEQ ID NO: 70. It encodes the amino acid sequence set out in SEQ ID NO: 69.

In some embodiments, the microorganism exhibits reduced metA gene expression or homoserine succinyltransferase activity in comparison to a corresponding wild-type microorganism. An exemplary DNA sequence of a metA coding sequence known in the art is set out in SEQ ID NO: 72. It encodes the amino acid sequence set out in SEQ ID NO: 71.

In some embodiments, the microorganism exhibits increased thrB gene expression or homoserine kinase activity in comparison to a corresponding wild-type microorganism. An exemplary DNA sequence of a thrB coding sequence known in the art is set out in SEQ ID NO: 74. It encodes the amino acid sequence set out in SEQ ID NO: 73.

In some embodiments, the microorganism exhibits increased thrC gene expression or threonine synthase activity in comparison to a corresponding wild-type microorganism. An exemplary DNA sequence of a thrC coding sequence known in the art is set out in SEQ ID NO: 76. It encodes the amino acid sequence set out in SEQ ID NO: 75.

In a twenty-first embodiment, the invention provides a method of culturing the further modified microorganisms to produce products of the invention.

### BRIEF DESCRIPTION OF THE DRAWINGS

FIG. 1 shows steps in the conversion of glucose to propionic acid via the threonine pathway.

FIG. 2 shows steps in the conversion of glucose to propionic acid via the citramalate pathway.

FIG. 3A shows steps in the conversion of glucose to propionic acid using the reductive portion of the tricarboxylic acid cycle. FIG. 3B illustrates the relative flux between the oxidative and reductive branch of the TCA cycle that is calculated to maximize the yield of propionic acid.

FIG. 4 shows LC-MS analysis of samples of propionyl-CoA after incubation of 2-ketobutyric acid with pyruvate dehydrogenase or 2-ketoglutarate dehydrogenase and the proper cofactors.

FIG. 5 shows production of propionic acid in cultured Escherichia coli engineered to convert threonine to propionic acid.

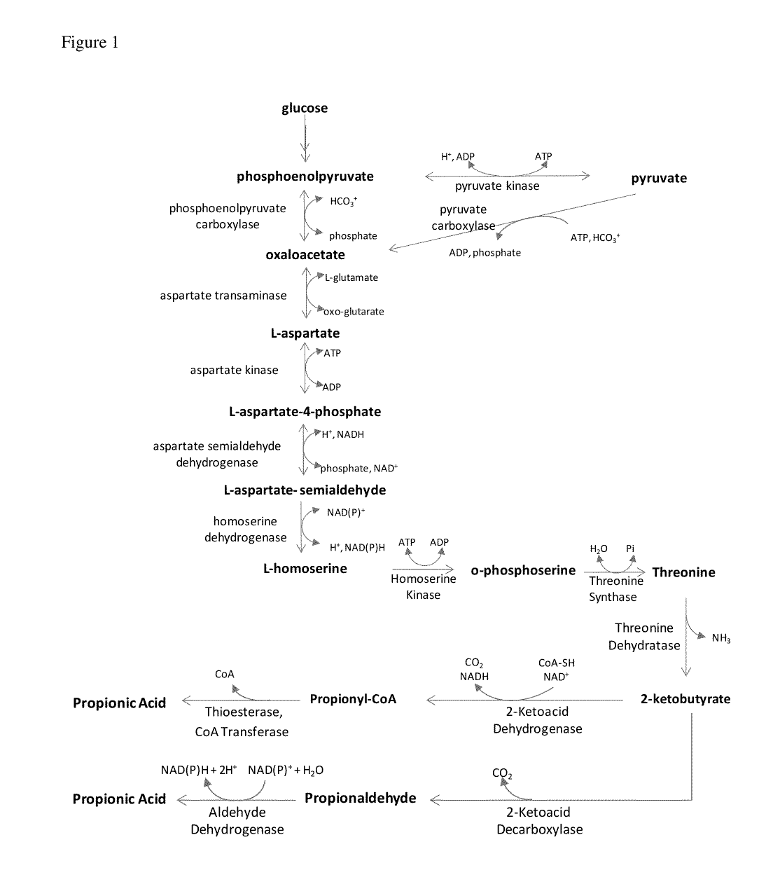

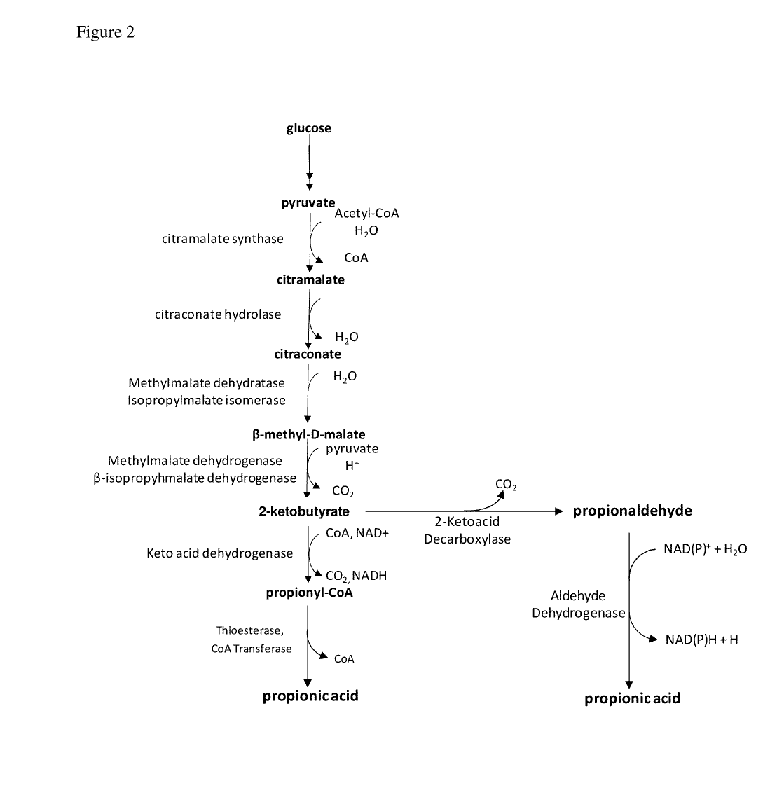

### DETAILED DESCRIPTION OF THE INVENTION

### Definitions

The invention provides the products propionic (propanoic) acid and propionate (propanoate). As is understood in the art propionate is the carboxylate anion (i.e., conjugate base) of propionic acid. The pH of the product solution determines the relative amount of propionate versus propionic acid in a preparation according to the Henderson-Hasselbalch equation {pH=pKa+log([A⁻][HA]}, where pKa is −log(Ka). Ka is the acid dissociation constant of propionic acid. The pKa of propionic acid in water is about 4.87. Thus, at or near neutral pH, propionic acid will exist primarily as the carboxylate anion. As used herein, “propionic (propanoic) acid” and “propionate (propanoate)” are both meant to encompass the other.

As used herein, “amplify,” “amplified,” or “amplification” refers to any process or protocol for copying a polynucleotide sequence into a larger number of polynucleotide molecules, e.g., by reverse transcription, polymerase chain reaction, and ligase chain reaction.

As used herein, an “antisense sequence” refers to a sequence that specifically hybridizes with a second polynucleotide sequence. For instance, an antisense sequence is a DNA sequence that is inverted relative to its normal orientation for transcription. Antisense sequences can express an

RNA transcript that is complementary to a target mRNA molecule expressed within the host cell (e.g., it can hybridize to target mRNA molecule through Watson-Crick base pairing).

As used herein, “cDNA” refers to a DNA that is complementary or identical to an mRNA, in either single stranded or double stranded form.

As used herein, “complementary” refers to a polynucleotide that base pairs with a second polynucleotide. Put another way, “complementary” describes the relationship between two single-stranded nucleic acid sequences that anneal by base-pairing. For example, a polynucleotide having the sequence 5′-GTCCGA-3′ is complementary to a polynucleotide with the sequence 5′-TCGGAC-3′.

As used herein, a “conservative substitution” refers to the substitution in a polypeptide of an amino acid with a functionally similar amino acid. Put another way, a conservative substitution involves replacement of an amino acid residue with an amino acid residue having a similar side chain. Families of amino acid residues having similar side chains have been defined within the art, and include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine) and aromatic side chains (e.g., tyrosine, phenylalanine, tryptophan, and histidine).

As used herein, “converting” means a chemical transformation from one molecule to another, primarily catalyzed by an enzyme or enzymes, although other organic or inorganic catalysts may be used, or the transformation can be spontaneous.

As used herein, a “corresponding wild-type microorganism” is the naturally-occurring microorganism that would be the same as the microorganism of the invention except that the naturally-occurring microorganism has not been genetically engineered to express any recombinant genes.

As used herein, “encoding” refers to the inherent property of nucleotides to serve as templates for synthesis of other polymers and macromolecules. Unless otherwise specified, a “nucleotide sequence encoding an amino acid sequence” includes all nucleotide sequences that are degenerate versions of each other and that encode the same amino acid sequence.

As used herein, “endogenous” refers to polynucleotides, polypeptides, or other compounds that are expressed naturally or originate within an organism or cell. That is, endogenous polynucleotides, polypeptides, or other compounds are not exogenous. For instance, an “endogenous” polynucleotide or peptide is present in the cell when the cell was originally isolated from nature.

As used herein, “expression vector” refers to a vector comprising a recombinant polynucleotide comprising expression control sequences operatively linked to a nucleotide sequence to be expressed. For example, suitable expression vectors can be an autonomously replicating plasmid or integrated into the chromosome.

As used herein, “exogenous” refers to any polynucleotide or polypeptide that is not naturally found or expressed in the particular cell or organism where expression is desired. Exogenous polynucleotides, polypeptides, or other compounds are not endogenous.

As used herein “threonine” includes enantiomers such as L-threonine ine and D-threonine.

As used herein, “hybridization” includes any process by which a strand of a nucleic acid joins with a complementary nucleic acid strand through base-pairing. Thus, the term refers to the ability of the complement of the target sequence to bind to a test (i.e., target) sequence, or vice-versa.

As used herein, “hybridization conditions” are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm−5° C. (5° below the Tm of the probe); “high stringency” at about 5-10° below the Tm; “intermediate stringency” at about 10-20° below the Tm of the probe; and “low stringency” at about 20-25° below the Tm.

Alternatively, or in addition, hybridization conditions can be based upon the salt or ionic strength conditions of hybridization and/or one or more stringency washes. For example, 6×SSC=very low stringency; 3×SSC=low to medium stringency; 1×SSC=medium stringency; and 0.5×SSC=high stringency. Functionally, maximum stringency conditions may be used to identify nucleic acid sequences having strict (i.e., about 100%) identity or near-strict identity with the hybridization probe; while high stringency conditions are used to identify nucleic acid sequences having about 80% or more sequence identity with the probe.

As used herein, “identical” or percent “identity,” in the context of two or more polynucleotide or polypeptide sequences, refers to two or more sequences that are the same or have a specified percentage of nucleotides or amino acid residues that are the same, when compared and aligned for maximum correspondence, as measured using sequence comparison algorithms or by visual inspection.

As used herein, “isolated enzyme” refers to enzymes free of a living organism. Isolated enzymes of the invention may be suspended in solution following lysing of the cell they were expressed in, partially or highly purified, soluble or bound to an insoluble matrix.

“Microorganisms” of the invention expressing recombinant genes are not naturally-occurring. In other words, the microorganisms are man-made and have been genetically engineered to express recombinant genes. The microorganisms of the invention have been genetically engineered to express the recombinant genes encoding the enzymes necessary to carry out the conversion of homoserine to the desired product. Microorganisms of the invention are bacteria, yeast or fungus. Bacteria include, but not limited to, Escherichia coli strains K, B or C. Microorganisms that are more resistant to propionate are preferred. Plant cells that are not naturally-occurring (are man-made) and have been genetically engineered to express recombinant genes carrying out the conversions detailed herein are contemplated by the invention to be alternative cells to microorganisms, for example in the production of poly-3-hydroxypropionate.

As used herein, “naturally-occurring” refers to an object that can be found in nature. For example, a polypeptide or polynucleotide sequence that is present in an organism (including viruses) that can be isolated from a source in nature and which has not been intentionally modified by man in the laboratory is naturally-occurring. As used herein, “naturally-occurring” and “wild-type” are synonyms.

As used herein, “operably linked,” when describing the relationship between two DNA regions or two polypeptide regions, means that the regions are functionally related to each other. For example, a promoter is operably linked to a coding sequence if it controls the transcription of the sequence; a ribosome binding site is operably linked to a coding sequence if it is positioned so as to permit translation; and a sequence is operably linked to a peptide if it functions as a signal sequence, such as by participating in the secretion of the mature form of the protein.

As used herein, a recombinant gene that is “over-expressed” produces more RNA and/or protein than a corresponding naturally-occurring gene in the microorganism. Methods of measuring amounts of RNA and protein are known in the art. Over-expression can also be determined by measuring protein activity such as enzyme activity. Depending on the embodiment of the invention, “over-expression” is an amount at least 3%, at least 5%, at least 10%, at least 20%, at least 25%, or at least 50% more. An over-expressed polynucleotide is generally a polynucleotide native to the host cell, the product of which is generated in a greater amount than that normally found in the host cell. Over-expression is achieved by, for instance and without limitation, operably linking the polynucleotide to a different promoter than the polynucleotide's native promoter or introducing additional copies of the polynucleotide into the host cell.

As used herein, “polynucleotide” refers to a polymer composed of nucleotides. The polynucleotide may be in the form of a separate fragment or as a component of a larger nucleotide sequence construct, which has been derived from a nucleotide sequence isolated at least once in a quantity or concentration enabling identification, manipulation, and recovery of the sequence and its component nucleotide sequences by standard molecular biology methods, for example, using a cloning vector. When a nucleotide sequence is represented by a DNA sequence (i.e., A, T, G, C), this also includes an RNA sequence (i.e., A, U, G, C) in which “U” replaces “T.” Put another way, “polynucleotide” refers to a polymer of nucleotides removed from other nucleotides (a separate fragment or entity) or can be a component or element of a larger nucleotide construct, such as an expression vector or a polycistronic sequence.

Polynucleotides include DNA, RNA and cDNA sequences.

As used herein, “polypeptide” refers to a polymer composed of amino acid residues which may or may not contain modifications such as phosphates and formyl groups.

As used herein, “primer” refers to a polynucleotide that is capable of specifically hybridizing to a designated polynucleotide template and providing a point of initiation for synthesis of a complementary polynucleotide when the polynucleotide primer is placed under conditions in which synthesis is induced.

As used herein, “recombinant polynucleotide” refers to a polynucleotide having sequences that are not joined together in nature. A recombinant polynucleotide may be included in a suitable vector, and the vector can be used to transform a suitable host cell. A host cell that comprises the recombinant polynucleotide is referred to as a “recombinant host cell.” The polynucleotide is then expressed in the recombinant host cell to produce, e.g., a “recombinant polypeptide.”

As used herein, “recombinant expression vector” refers to a DNA construct used to express a polynucleotide that, e.g., encodes a desired polypeptide. A recombinant expression vector can include, for example, a transcriptional subunit comprising (i) an assembly of genetic elements having a regulatory role in gene expression, for example, promoters and enhancers, (ii) a structural or coding sequence which is transcribed into mRNA and translated into protein, and (iii) appropriate transcription and translation initiation and termination sequences. Recombinant expression vectors are constructed in any suitable manner. The nature of the vector is not critical, and any vector may be used, including plasmid, virus, bacteriophage, and transposon. Possible vectors for use in the invention include, but are not limited to, chromosomal, nonchromosomal and synthetic DNA sequences, e.g., bacterial plasmids; phage DNA; yeast plasmids; and vectors derived from combinations of plasmids and phage DNA, DNA from viruses such as vaccinia, adenovirus, fowl pox, baculovirus, SV40, and pseudorabies.

As used herein, a “recombinant gene” is not a naturally-occurring gene. A recombinant gene is man-made. A recombinant gene includes a protein coding sequence operably linked to expression control sequences. Embodiments include, but are not limited to, an exogenous gene introduced into a microorganism, an endogenous protein coding sequence operably linked to a heterologous promoter (i.e., a promoter not naturally linked to the protein coding sequence) and a gene with a modified protein coding sequence (e.g., a protein coding sequence encoding an amino acid change or a protein coding sequence optimized for expression in the microorganism). The recombinant gene is maintained in the genome of the microorganism, on a plasmid in the microorganism or on a phage in the microorganism.

As used herein, “reduced” expression is expression of less RNA or protein than the corresponding natural level of expression. Methods of measuring amounts of RNA and protein are known in the art. Reduced expression can also be determined by measuring protein activity such as enzyme activity. Depending on the embodiment of the invention, “reduced” is an amount at least 3%, at least 5%, at least 10%, at least 20%, at least 25%, or at least 50% less.

As used herein, “specific hybridization” refers to the binding, duplexing, or hybridizing of a polynucleotide preferentially to a particular nucleotide sequence under stringent conditions.

As used herein, “stringent conditions” refers to conditions under which a probe will hybridize preferentially to its target subsequence, and to a lesser extent to, or not at all to, other sequences.

As used herein, “substantially homologous” or “substantially identical” in the context of two nucleic acids or polypeptides, generally refers to two or more sequences or subsequences that have at least 40%, 60%, 80%, 90%, 95%, 96%, 97%, 98% or 99% nucleotide or amino acid residue identity, when compared and aligned for maximum correspondence, as measured using sequence comparison algorithms or by visual inspection. The substantial identity can exist over any suitable region of the sequences, such as, for example, a region that is at least about 50 residues in length, a region that is at least about 100 residues, or a region that is at least about 150 residues. In certain embodiments, the sequences are substantially identical over the entire length of either or both comparison biopolymers.

Polynucleotides

The polynucleotide(s) encoding one or more enzyme activities for steps in the pathways of the invention may be derived from any source. Depending on the embodiment of the invention, the polynucleotide is isolated from a natural source such as bacteria, algae, fungi, plants, or animals; produced via a semi-synthetic route (e.g., the nucleic acid sequence of a polynucleotide is codon optimized for expression in a particular host cell, such as Escherichia coli); or synthesized de novo. In certain embodiments, it is advantageous to select an enzyme from a particular source based on, e.g., the substrate specificity of the enzyme or the level of enzyme activity in a given host cell. In some embodiments of the invention, the enzyme and corresponding polynucleotide are naturally found in the host cell and over-expression of the polynucleotide is desired. In this regard, in some embodiments, additional copies of the polynucleotide are introduced in the host cell to increase the amount of enzyme. In some embodiments, over-expression of an endogenous polynucleotide may be achieved by upregulating endogenous promoter activity, or operably linking the polynucleotide to a more robust heterologous promoter.

Exogenous enzymes and their corresponding polynucleotides also are suitable for use in the context of the invention, and the features of the biosynthesis pathway or end product can be tailored depending on the particular enzyme used.

The invention contemplates that polynucleotides of the invention may be engineered to include alternative degenerate codons to optimize expression of the polynucleotide in a particular microorganism. For example, a polynucleotide may be engineered to include codons preferred in Escherichia coli if the DNA sequence will be expressed in Escherichia coli. Methods for codon-optimization are known in the art.

Enzyme Variants

In certain embodiments, the microorganism produces an analog or variant of the polypeptide encoding an enzyme activity. Amino acid sequence variants of the polypeptide include substitution, insertion, or deletion variants, and variants may be substantially homologous or substantially identical to the unmodified polypeptides. In certain embodiments, the variants retain at least some of the biological activity, e.g., catalytic activity, of the polypeptide. Other variants include variants of the polypeptide that retain at least about 50%, preferably at least about 75%, more preferably at least about 90%, of the biological activity. Substitutional variants typically exchange one amino acid for another at one or more sites within the protein. Substitutions of this kind can be conservative, that is, one amino acid is replaced with one of similar shape and charge. Conservative substitutions include, for example, the changes of: alanine to serine; arginine to lysine; asparagine to glutamine; aspartate to glutamate; cysteine to serine; glutamine to asparagine; glutamate to aspartate; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. An example of the nomenclature used herein to indicate an amino acid substitution is “S345F ThrA” wherein the naturally occurring serine occurring at position 345 of the naturally occurring ThrA enzyme which has been substituted with a phenylalanine.

In some instances, the microorganism comprises an analog or variant of the exogenous or over-expressed polynucleotide(s) described herein. Nucleic acid sequence variants include one or more substitutions, insertions, or deletions, and variants may be substantially homologous or substantially identical to the unmodified polynucleotide. Polynucleotide variants or analogs encode mutant enzymes having at least partial activity of the unmodified enzyme. Alternatively, polynucleotide variants or analogs encode the same amino acid sequence as the unmodified polynucleotide. Codon optimized sequences, for example, generally encode the same amino acid sequence as the parent/native sequence but contain codons that are preferentially expressed in a particular host organism.

A polypeptide or polynucleotide “derived from” an organism contains one or more modifications to the naturally-occurring amino acid sequence or nucleotide sequence and exhibits similar, if not better, activity compared to the native enzyme (e.g., at least 70%, at least 80%, at least 90%, at least 95%, at least 100%, or at least 110% the level of activity of the native enzyme). For example, enzyme activity is improved in some contexts by directed evolution of a parent/naturally-occurring sequence. Additionally or alternatively, an enzyme coding sequence is mutated to achieve feedback resistance.

In some instances, the selected microorganism is modified to increase carbon flux through the metabolic pathway from glucose to propionyl-CoA, and example being the high flux through the threonine pathway engineered in Escherichia coli (Lee, et. al, Molecular Systems Biology, 3: article 149 (2007).

Expression Vectors/Transfer into Microorganisms

Expression vectors for recombinant genes can be produced in any suitable manner to establish expression of the genes in a microorganism. Expression vectors include, but are not limited to, plasmids and phage. The expression vector can include the exogenous polynucleotide operably linked to expression elements, such as, for example, promoters, enhancers, ribosome binding sites, operators and activating sequences. Such expression elements may be regulatable, for example, inducible (via the addition of an inducer). Alternatively or in addition, the expression vector can include additional copies of a polynucleotide encoding a native gene product operably linked to expression elements. Representative examples of useful heterologous promoters include, but are not limited to: the LTR (long terminal 35 repeat from a retrovirus) or SV40 promoter, the Escherichia coli lac, tet, or trp promoter, the phage Lambda PL promoter, and other promoters known to control expression of genes in prokaryotic or eukaryotic cells or their viruses. In one aspect, the expression vector also includes appropriate sequences for amplifying expression. The expression vector can comprise elements to facilitate incorporation of polynucleotides into the cellular genome.

Introduction of the expression vector or other polynucleotides into cells can be performed using any suitable method, such as, for example, transformation, electroporation, microinjection, microprojectile bombardment, calcium phosphate precipitation, modified calcium phosphate precipitation, cationic lipid treatment, photoporation, fusion methodologies, receptor mediated transfer, or polybrene precipitation. Alternatively, the expression vector or other polynucleotides can be introduced by infection with a viral vector, by conjugation, by transduction, or by other suitable methods.

Culture

Microorganisms of the invention comprising recombinant genes are cultured under conditions appropriate for growth of the cells and expression of the gene(s). Microorganisms expressing the polypeptide(s) can be identified by any suitable methods, such as, for example, by PCR screening, screening by Southern blot analysis, or screening for the expression of the protein. In some embodiments, microorganisms that contain the polynucleotide can be selected by including a selectable marker in the DNA construct, with subsequent culturing of microorganisms containing a selectable marker gene, under conditions appropriate for survival of only those cells that express the selectable marker gene. The introduced DNA construct can be further amplified by culturing genetically modified microorganisms under appropriate conditions (e.g., culturing genetically modified microorganisms containing an amplifiable marker gene in the presence of a concentration of a drug at which only microorganisms containing multiple copies of the amplifiable marker gene can survive).

In some embodiments, the microorganisms (such as genetically modified bacterial cells) have an optimal temperature for growth, such as, for example, a lower temperature than normally encountered for growth and/or fermentation. In addition, in certain embodiments, cells of the invention exhibit a decline in growth at higher temperatures as compared to normal growth and/or fermentation temperatures as typically found in cells of the type.

Any cell culture condition appropriate for growing a microorganism and synthesizing a product of interest is suitable for use in the inventive method.

Recovery

The methods of the invention optionally comprise a step of product recovery. Recovery of propionate can be carried out by methods known in the art. For example, propionate can be recovered by distillation methods, extraction methods, crystallization methods, or combinations thereof.

### EXAMPLES

The following examples further describe and demonstrate embodiments within the scope of the present invention. The examples are given solely for the purpose of illustration and are not to be construed as limiting the present invention. Example 1 describes expression vectors for branched-chain alpha-ketoacid decarboxylase (KdcA); Example 2 describes expression vectors for Coenzyme-A acylating propionaldehyde dehydrogenase (PduP); Example 3 describes expression vectors for Acyl-CoA Thioesterase (TesB); Example 4 describes the transformation of E. coli; Example 5 describes the culturing of the Escherichia coli; Example 6 describes the isolation of expressed proteins; Example 7 describes in vitro production of propionyl-CoA with 2-ketoacid dehydrogenases; Example 8 describes increasing propionyl-CoA production by increasing carbon flow through the threonine-dependent pathway; Example 9 describes increasing 2-keto butyrate production by increasing carbon flow through the citramalate-dependent pathway; Example 10 describes the analytical procedures for the measurement of 2-ketobutyric acid, propionyl-CoA and propionic acid; Example 11 describes the production of propionic acid in engineered Escherichia coli.

### Example 1

### Expression Vector for Branched-Chain Alpha-Ketoacid Decarboxylase (KdcA)

An Escherichia coli expression vector was constructed for production of a recombinant branched-chain alpha-ketoacid decarboxylase (KdcA) gene. A common cloning strategy was established utilizing the modified pET30a-BB vector providing for T7 promoter control and His-tagged recombinant proteins. Lactococcus lactis branched-chain alpha-ketoacid decarboxylase gene was codon-optimized for expression in Escherichia coli and synthesized (GenScript, Piscataway, N.J.). To facilitate cloning and expression, the synthesis design included the addition of EcoRI, NotI, XbaI restriction sites and a Ribosomal Binding Site (RBS) 5′ to the ATG start codon, and SpeI, NotI and PstI restriction sites 3′ to the stop codon. The branched-chain alpha-ketoacid decarboxylase gene sequence was further optimized by the removal of the common restriction sites: AvrII; BamHI; BglII; BstBI; EagI; EcoRI; EcoRV; HindIII; KpnI; NcoI; NheI; NotI; NspV; PstI; PvuII; SacI; SalI; SapI; SfuI; SpeI; XbaI; XhoI (SEQ ID NO: 34). The optimized sequence was cloned into the pET30a-BB vector at the EcoRI and PstI sites. The resulting expression vector was designated pET30a-BB Ll KDCA and the enzyme encoded (SEQ ID NO: 33).

### Example 2

### Expression Vector for Coenzyme-A Acylating Propionaldehyde Dehydrogenase (PduP)

An Escherichia coli expression vector was constructed for production of a recombinant Coenzyme-A acylating propionaldehyde dehydrogenase (PduP) gene. A common cloning strategy was established utilizing the modified pET30a-BB vector providing for T7 promoter control and His-tagged recombinant proteins. Salmonella enterica Coenzyme-A acylating propionaldehyde dehydrogenase gene was codon-optimized for expression in Escherichia coli and synthesized (GenScript, Piscataway, N.J.). To facilitate cloning and expression, the synthesis design included the addition of EcoRI, Nod, XbaI restriction sites and a Ribosomal Binding Site (RBS) 5′ to the ATG start codon, and SpeI, NotI and PstI restriction sites 3′ to the stop codon. The Coenzyme-A acylating propionaldehyde dehydrogenase gene sequence was further optimized by the removal of the common restriction sites: AvrII; BamHI; BglII; BstBI; EagI; EcoRI; EcoRV; HindIII; KpnI; NcoI; NheI; NotI; NspV; PstI; PvuII; SacI; SalI; SapI; SfuI; SpeI; XbaI; XhoI (SEQ ID NO: 36). The optimized sequence was cloned into the pET30a-BB vector at the EcoRI and PstI sites. The resulting expression vector was designated pET30a-BB Se PDUP and the enzyme encoded (SEQ ID NO: 35).

### Example 3

### Expression Vectors for Acyl-CoA Thioesterase Gene (tesB)

An Escherichia coli expression vector was constructed for production of a recombinant short to medium-chain acyl-CoA thioesterase gene. A common cloning strategy was established utilizing the pET30a vector (Novagen [EMD Chemicals, Gibbstown, N.J.] #69909-30) providing for T7 promoter control and His-tagged recombinant proteins. Escherichia coli acyl-CoA thioesterase II (TesB) gene was codon optimized for expression in Escherichia coli and synthesized (GenScript, Piscataway, N.J.). To facilitate cloning, the synthesis design included the addition of BamHI and XbaI restriction sites 5′ to the ATG start codon, and SacI and HindIII restriction sites 3′ to the stop codon. The thioesterase gene sequences were further optimized by the removal of the common restriction sites: BamHI, BglII, BstBI, EcoRI, HindIII, KpnI, PstI, NcoI, NotI, SacI, SalI, XbaI, and XhoI (SEQ ID NO: 2). The optimized sequences were cloned into the pET30a vector at the BamHI and SacI sites. The resulting expression vector was designated pET30a Ec TesB and the enzyme encoded (SEQ ID NO: 1).

### Example 4

### Transformation of E. coli

The recombinant plasmids were then used to transform chemically competent One ShotBL21 (DE3) pLysS Escherichia coli cells (Invitrogen, Carlsbad, Calif.). Individual vials of cells were thawed on ice and gently mixed with 10 μs of plasmid DNA. The vials were incubated on ice for 30 minutes. The vials were briefly incubated at 42° C. for 30 seconds and quickly replaced back on ice for an additional 2 minutes. 250 μl of 37° C. SOC medium was added and the vials were secured horizontally on a shaking incubator platform and incubated for 1 hour at 37° C., 225 rpm. Aliquots of 20 μl and 200 μl cells were plated onto selective LB agar (50 μg/ml kanamycin; 34 μg/ml chloramphenicol) plates to select for cells carrying the recombinant and pLysS plasmids respectively and incubated overnight at 37° C. Single colony isolates were isolated, cultured in 5 ml of selective LB broth and recombinant plasmids were isolated using a QIAPrep® Spin Miniprep Kit (Qiagen, Valencia, Calif.) spin plasmid miniprep kit. Plasmid DNAs were characterized by gel electrophoresis of restriction digests with AflIII.

### Example 5

### Culture of Escherichia coli

Overnight cultures of transformed strains (15 ml of LB broth; 34 μg/ml chloramphenicol; 50 μg/ml kanamycin) in 50 ml conical tubes were inoculated from a loop full of frozen glycerol stocks. Cultures were incubated overnight at 25° C. with 250 rpm shaking. LB broth (500 ml, containing 34 μg/ml chloramphenicol, 50 μg/ml kanamycin; equilibrated to 25° C.) in 2.8 L fluted Erlenmeyer flasks was inoculated from the overnight cultures at an optical density (OD) at 600 nm of ˜0.1. Cultures were continued at 25° C. with 250 rpm shaking and optical density monitored until A₆₀₀ of ˜0.4. Plasmid recombinant gene protein expression was then induced by addition of 500 μL of 1M IPTG (Teknova, Hollister, Calif.; 1 mM final concentration). Cultures were further incubated for 24 hours at 25° C. with 250 rpm shaking before the cells were collected by centrifugationn and the pellets stored at −80° C.

### Example 6

### Recombinant Protein Isolation

His-tagged recombinant proteins were isolated by metal chelate affinity/gravity-flow chromatography utilizing nickel-nitrilotriacetic acid coupled Sepharose CL-6B resin (Ni-NTA, Qiagen, Valencia, Calif.) as follows: Cell pellets were thawed on ice and suspended in 20 ml of a 20 mM sodium phosphate, 500 mM NaCl, 20 mM imidazole (pH 7.4) binding buffer (with 1 mg/mL lysozyme and 1 Complete EDTA-free protease inhibitor pellet [Roche Applied Science, Indianapolis, Ind.]. Samples were incubated at 4° C. with 30 rpm rotation for 30 minutes. Cell lysates were disrupted 2× in a Thermo French Press; 1 inch cylinder; 1000 psi. Cell debris was pelleted by centrifugation for 1 hour at 15,000×g, 4° C. The supernatant was transferred to a 5 ml column bed of Ni-NTA equilibrated in binding buffer (20 mM sodium phosphate, 500 mM NaCl, 20 mM imidazole, pH 7.4). The Ni-NTA was suspended in the supernatant and incubated for 60 minutes with slow rocker mixing at 4° C. The bound media was then washed by gravity flow of 20× bed volumes (100 ml) of binding buffer followed by 10× bed volumes (50 ml) of rinse buffer (20 mM sodium phosphate, 500 mM NaCl, 100 mM imidazole, pH 7.4). Bound proteins were eluted by gravity-flow in 10× bed volumes (50 ml) of elution buffer (20 mM sodium phosphate, 500 mM NaCl, 500 mM imidazole, pH 7.4) and collected in fractions. Fraction samples were assayed for protein by SDS-PAGE analysis, pooled, and concentrated with Amicon Ultra-15 Centrifugal Filter Devices (EMD Millipore, Billerica, Mass.) with a 30K nominal molecular weight limit. The concentrated protein isolates were desalted and eluted into 3.5 ml of storage buffer (50 mM HEPES (pH 7.3-7.5); 300 mM NaCl; 20% glycerol) using PD-10 Desalting Columns (GE Healthcare Biosciences, Pittsburgh, Pa.)

### Example 7

### In Vitro Production of Propionyl-CoA with 2-Ketoacid Dehydrogenases

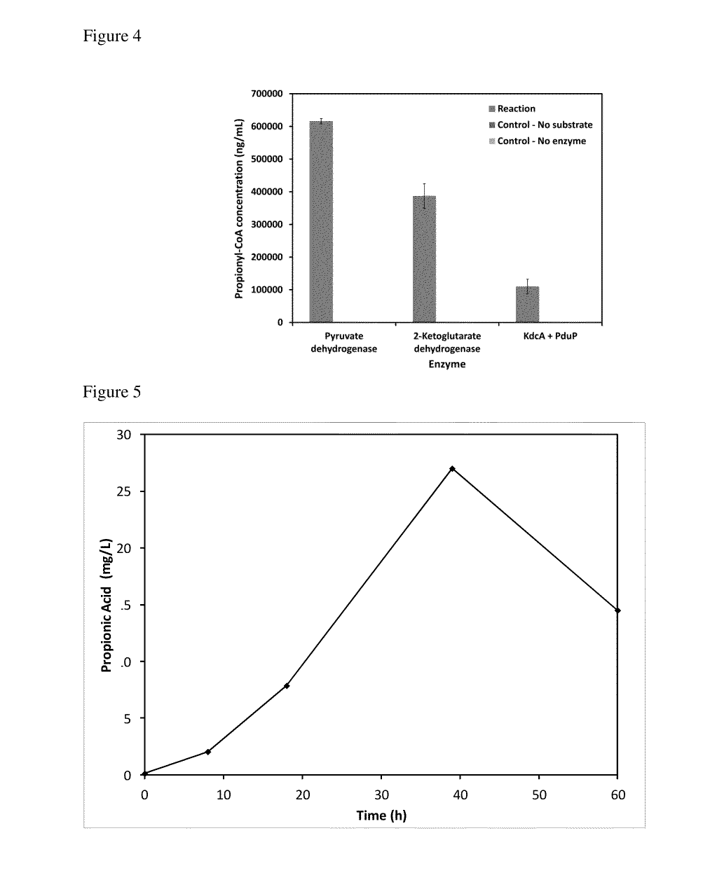

In a first assay, 2-ketobutyric acid (2 mM) was incubated with or without commercial porcine heart pyruvate dehydrogenase (1.4 mg/mL, Sigma) in the presence of coenzyme A (2 mM), β-NAD⁺ (2 mM), thiamine pyrophosphate (0.2 mM), MgCl₂ (2 mM), and HEPES buffer (50 mM, pH 7.3). In a second assay, pyruvate dehydrogenase was substituted for porcine heart 2-ketoglutarate dehydrogenase (1.0 mg/mL, Sigma) while keeping the other components. In a third assay, purified 2-ketoacid decarboxylase KdcA (1.8 μm) and propionaldehyde dehydrogenase PduP (1.8 μm) were used. The samples were incubated at room temperature for 17 h, followed by LC-MS analysis to determine concentrations of propionyl-CoA. Only when the dehydrogenases (and decarboxylase) were present, the product was detected in significant amounts (FIG. 4).

### Example 8

### Increasing Propionyl-CoA Production by Increasing Carbon Flow Through the Threonine-Dependent Pathway

This example demonstrates that increasing carbon flow through a pathway utilizing threonine increases propionyl-CoA production in host cells. An Escherichia coli strain was modified to increase production of threonine deaminase. Threonine deaminase promotes the conversion of threonine to 2-ketobutyrate. An expression vector comprising an Escherichia coli threonine deaminase coding sequence, tdcB, operably linked to a trc promoter was constructed. To isolate tdcB, genomic DNA was prepared from Escherichia coli BW25113 (Escherichia coli Genetic Stock Center, Yale University, New Haven, Conn.) by picking an isolated colony from a Luria agar plate, suspending the colony in 100 μl Tris (1 mM; pH 8.0), 0.1 mM EDTA, boiling the sample for five minutes, and removing the insoluble debris by centrifugation. tdcB was amplified from the genomic DNA sample by PCR using primers GTGCCATGGCTCATA TTACATACGATCTGCCGGTTGC (SEQ ID NO: 77) and GATCGAATTCATCCTTAGGCGTCAACGAAACCGGTGATTTG (SEQ ID NO: 78). PCR was performed on samples having 1 μl of Escherichia coli BW25113 genomic DNA, 1 μl of a 10 μM stock of each primer, 25 μl of Pfu Ultra II Hotstart 2× master mix (Agilent Technologies, Santa Clara, Calif.), and 22 μl of water. PCR conditions were as follows: the samples were initially incubated at 95° C. for two minutes, followed by three cycles at 95° C. for 20 seconds (strand separation), 56° C. for 20 seconds (primer annealing), and 72° C. primer extension for 30 seconds. In addition, 27 cycles were run at 95° C. for 20 seconds, 60° C. for 20 seconds, and 72° C. primer extension for 30 seconds. There was a three minute incubation at 72° C., and the samples were held at 4° C.

The PCR products were purified using a QIAquick® PCR Purification Kit (Qiagen), double digested with restriction enzymes HindIII and NcoI, and ligated (Fast-Link Epicentre Biotechnologies, Madison, Wis.) with HindIII/NcoI-digested pTrcHisA vector (Invitrogen, Carlsbad, Calif.). The ligation mix was used to transform OneShot Top10™ Escherichia coli cells (Invitrogen, Carlsbad, Calif.). Individual vials of cells were thawed on ice and gently mixed with 2 μl of ligation mix. The vials were incubated on ice for 30 minutes. The vials were briefly incubated at 42° C. for 30 seconds and quickly replaced back on ice for an additional 2 minutes. 250 μl of 37° C. SOC medium was added and the vials were secured horizontally on a shaking incubator platform and incubated for 1 hour at 37° C., 225 rpm. Aliquots of 20 μl and 200 μl cells were plated onto selective LB agar (100 μg/ml ampicillin). Single colony isolates were isolated, cultured in 50 ml of selective LB broth and the recombinant plasmid was isolated using a Qiagen HiSpeed Plasmid Midi Kit and characterized by gel electrophoresis of restriction digests with HindIII and NcoI. DNA sequencing confirmed that the tdcB insert had been cloned and that the insert encoded the published amino acid sequence (Genbank number U00096.2) (SEQ ID NOs: 16 and 15). The resulting plasmid was designated pTrcHisA Ec tdcB.

### Example 9

### Increasing 2-Keto Butyrate Production by Increasing Carbon Flow Through the Citramalate-Dependent Pathway

This example describes the generation of a recombinant microbe that produces exogenous citramalate synthase to further increase 2-keto butyrate production. A Methanococcus jannaschii citramalate synthase gene was codon optimized for enzyme activity in Escherichia coli (Atsumi et al., Applied and Environmental Microbiology 74: 7802-8 (2008)). The native M. jannaschii citramalate synthase coding sequence also was mutated through directed evolution to improve enzyme activity and feedback resistance. Escherichia coli is not known to have citramalate synthase activity, and a strain was engineered to produce exogenous citramalate synthase while overproducing three native Escherichia coli enzymes: LeuB, LeuC, and LeuD. Citramalate synthase, LeuB, LeuC, and LeuD mediate the first four chemical conversions in the citramalate pathway to produce 2-keto butyrate.

To generate a synthetic CimA3.7 gene codon-optimized for Escherichia coli expression, a DNA fragment (SEQ ID NO: 79) coding for the amino acid sequence (SEQ ID NO: 80) containing a restriction site BspHI (bases 1-6), codon-optimized cimA3. 7 fragment (bases 3-1118), stop codon TGA (bases 1119-1121), a fragment of 52 bases from the start of the Escherichia coli leuB gene (bases 1121-1173), and a linker sequence (bases 1174-1209) containing NotI, PacI, PmeI, XbaI and EcoRI sites was synthesized (GenScript, Piscataway, N.J.). The stop codon of cimA3.7 (TGA) and start codon (ATG) of leuB overlaps one base (A), presumably to enable translational coupling. This overlap mimics the native leuA and leuB coupling in Escherichia coli. The synthesized fragment was digested with BspHI and EcoRI and cloned into pTricHisA (Invitrogen) at the NcoI and EcoRI sites, using the compatible ends generated by BspHI and NcoI. The end of the leuB fragment (bases 1168-1173) also contains a BspEI site for cloning for leuBCD. This vector was designated as pTrcHisA Mj cimA.

To fuse the three-gene complex leuBCD behind M. jannaschii cimA, E. coli leuBCD cDNA was amplified from an Escherichia coli BW25113 genomic DNA sample using PCR primers (SEQ ID NO: 81 and SEQ ID NO: 82), which included a BspEI restriction site in leuB and incorporated a NotI restriction site 3′ of the stop codon of leuD during the PCR reaction. The PCR was performed with 50 μl of Pfu Ultra II Hotstart 2× master mix (Agilent Technologies, Santa Clara, Calif.), 1 μl of a mix of the two primers (10 μmoles of each), 1 μl of E. coli BW25113 genomic DNA, and 48 μl of water. The PCR began with a two minute incubation at 95° C., followed by 30 cycles of 20 seconds at 95° C. for denaturation, 20 seconds for annealing at 64° C., and two minutes at 72° C. for extension. The sample was incubated at 72° C. for three minutes and then held at 4° C. The PCR product (leuBCD insert, SEQ ID NO: 83) was purified using a QIAquick® PCR Purification Kit (Qiagen, Valencia, Calif.).

The leuBCD insert and the bacterial expression vector pTrcHisA Mj cimA were digestedwith BspEI. The digested vector and leuBCD insert were again purified using a QIAquick® PCR purification columns prior to being restriction digested with NotI. Following final column purification, the digested vector and insert were ligated using Fast-Link (Epicentre Biotechnologies, Madison, Wis.). The ligation mix was then used to transform Escherichia coli TOP10 cells (Invitrogen, Carlsbad, Calif.). Individual vials of cells were thawed on ice and gently mixed with 2 μl of ligation mix. The vials were incubated on ice for 30 minutes. The vials were briefly incubated at 42° C. for 30 seconds and quickly replaced back on ice for an additional 2 minutes. 250 μl of 37° C. SOC medium was added and the vials were secured horizontally on a shaking incubator platform and incubated for 1 hour at 37° C., 225 rpm. Aliquotes of 20 μl and 200 μl cells were plated onto selective LB agar (100 μg/ml ampicillin). Single colony isolates were isolated, cultured in 5 ml of selective LB broth and the recombinant plasmids were isolated using a QIAPrep® Spin Miniprep Kit (Qiagen) and characterized by gel electrophoresis of restriction digests with AflIII. DNA sequencing confirmed that the leuBCD insert had been cloned and that the insert encoded the published amino acid sequences (GenBank Accession No. AAC73184 (Ec leuB) (SEQ ID NO: 84); GenBank Accession No. AAC73183 (Ec leuC) (SEQ ID NO: 85); and GenBank Accession No. AAC73182 (Ec leuD) (SEQ ID NO: 86). The resulting plasmid was designated pTrc Mj cimA Ec leuBCD.

### Example 10

### Acyl-CoA and Organic Acid Assays for Cell Cultures

Coenzyme-A Analysis Sample Processing

Samples were prepared for CoA analysis. A stable-labeled (deuterium) internal standard containing master mix is prepared, comprising d₃-3-hydroxymethylglutaryl-CoA (200 μl of 60 μg/ml stock in 10 ml of 15% trichloroacetic acid). An aliquot (500 μl) of the master mix is added to a 2-ml microcentrifuge tube. Silicone oil (AR200; Sigma catalog number 85419; 700 μl) is layered onto the master mix. An Escherichia coli culture (700 μl) is layered gently on top of the silicone oil. The sample is subject to centrifugation at 20,000 g for five minutes at 4° in an Eppendorf 5417C centrifuge. A portion (˜240 μl) of the master mix-containing layer (lower layer) is transferred to an empty tube and frozen on dry ice for 30 minutes prior to storage at at −80° C.

Culture Broth Processing for 2-Ketobyric Acid and Propanoic Acid Analyses

Culture samples were processed for metabolite analysis as follows: Cells were pelleted by centrifugation at 5000×g; 4° C. Supernatants were filtered through Acrodisc Syringe Filters (0.2 μm HT Tuffryn membrane; low protein binding; Pall Corporation, Ann Arbor, Mich.) and frozen on dry ice prior to storage at at −80° C.

Measurement of Acyl-CoA Levels

The following method was used to prepare samples for acyl-CoA analysis. A stable-labeled (deuterium) internal standard-containing master mix was prepared, comprising d₃-3-hydroxymethylglutaryl-CoA (Cayman Chemical Co., 200 μl of 50 μg/ml stock in 10 ml of 15% trichloroacetic acid). An aliquot (500 μl) of the master mix was added to a 2-ml tube. Silicone oil (AR200; Sigma catalog number 85419; 800 μl) was layered onto the master mix. Clarified E. coli culture broth (800 μl) was layered gently on top of the silicone oil. The sample was subjected to centrifugation at 20,000 g for five minutes at 4° in an Eppendorf 5417C centrifuge. A portion (300 μl) of the master mix-containing layer was transferred to an empty tube and frozen on dry ice for 30 minutes.

The acyl-CoA content of samples was determined using LC/MS/MS. Individual CoA standards (CoA and acetyl-CoA) were purchased from Sigma Chemical Company (St. Louis, Mo.) and prepared as 500 μg/ml stocks in methanol. Acryloyl-CoA was synthesized and prepared similarly. The analytes were pooled, and standards with all of the analytes were prepared by dilution with 15% trichloroacetic acid. Standards for regression were prepared by transferring 500 μl of the working standards to an autosampler vial containing 10 μL of the 50 μg/ml internal standard. Sample peak areas (or heights) were normalized to the stable-labeled internal standard (d₃-3-hydroxymethylglutaryl-CoA,). Samples were assayed by HPLC/MS/MS on a Sciex API5000 mass spectrometer in positive ion Turbo Ion Spray. Separation was carried out by reversed-phase high performance liquid chromatography using a Phenomenex Onyx Monolithic C18 column (2×100 mm) and mobile phases of 1) 5 mM ammonium acetate, 5 mM dimethylbutylamine, 6.5 mM acetic acid and 2) acetonitrile with 0.1% formic acid, with the following gradient at a flow rate of 0.6 ml/min:

| | Mobile | Mobile |
|---|---|---|
| | Phase A | Phase B |
| Time | (%) | (%) |
| 0 min | 97.5 | 2.5 |
| 1.0 min | 97.5 | 2.5 |
| 2.5 min | 91.0 | 9.0 |
| 5.5 min | 45 | 55 |
| 6.0 min | 45 | 55 |
| 6.1 min | 97.5 | 2.5 |
| 7.5 min | — | — |
| 9.5 min | End Run | |

The conditions on the mass spectrometer were: DP 160, CUR 30, GS1 65, GS2 65, IS 4500, CAD 7, TEMP 650 C. The following transitions were used for the multiple reaction monitoring (MRM):

| | Precursor | Product | Collision | |
|---|---|---|---|---|
| Compound | Ion* | Ion* | Energy | CXP |
| n-Propionyl-CoA | 824.3 | 317.2 | 41 | 32 |
| Succinyl-CoA | 868.2 | 361.1 | 49 | 38 |
| Iso-Butyrl-CoA | 838.3 | 331.2 | 43 | 21 |
| Lactoyl-CoA | 840.3 | 333.2 | 45 | 38 |
| Acroyl-CoA | 822.4 | 315.4 | 45 | 36 |
| CoA | 768.3 | 261.2 | 45 | 34 |
| Isovaleryl-CoA | 852.2 | 345.2 | 45 | 34 |
| Malonyl-Coa | 854.2 | 347.2 | 41 | 36 |
| Acetyl-CoA | 810.3 | 303.2 | 43 | 30 |
| d3-3- | 915.2 | 408.2 | 49 | 13 |
| Hydroxymethyl- | | | | |
| glutaryl-CoA | | | | |

*Energies, in volts, for the MS/MS analysis

2-Ketobutyric Acid and Threonine Determination by Liquid Chromatography/Mass Spectometry

The 2-ketobutyrate and threonine content of samples was determined using LC/MS/MS. threonine standard was purchased from Sigma Chemical Company (St. Louis, Mo.) and a 2-ketobutryate standard obtained from Sigma-Aldrich. Stocks were prepared at 1.0 mg/ml in 50/50 methanol/water then standards of individual analtyes were prepared by dilution with 50/50 acetonitrile/water. Standards for regression were prepared by transferring 1.0 ml of the working standards to an autosampler vial containing 25 μL of the 20 μg/ml internal standard (L-threonine U13C4 UD5 15N and 2-ketobutyric Acid 13C4 3,3-D2) Samples were prepared by a 1:10 diultion was prepared by taking 100 μL of sample to a vial with 25 μL IS and 900 μL of 50:50 acetonitrile/water, cap and vortex to mix.

Sample peak areas were normalized to the stable-labeled internal standard for each analyte. Samples were assayed by HPLC/MS/MS on a Sciex API5000 mass spectrometer in positive ion Turbo Ion Spray. Separation was carried out by reversed-phase high performance liquid chromatography using a ZIC-HILIC, 2.1×50 mm, 5-μm particles and mobile phases of 1) 0.754% formic acid in water and 2) acetonitrile with 0.754% formic acid, with the following gradient at a flow rate of 0.35 ml/min:

| | Mobile | Mobile |
|---|---|---|
| | Phase A | Phase B |
| Time | (%) | (%) |
| 0 min | 97.5 | 95 |
| 1.0 min | 97.5 | 95 |
| 4.0 min | 91.0 | 5 |
| 5.0 min | 45 | 5 |
| 5.1 min | 45 | 95 |
| 9.0 min | End Run | |

The mass spectrometer was run in a two period mode with the first period configured in negative ionization to determine 2-ketobutryate and corresponding internal standard. The conditions on the mass spectrometer were: DP −60, CUR 30, GS1 60, GS2 60, IS −3500, CAD 12, TEMP 500 C. The following transitions were used for the multiple reaction monitoring (MRM):

| | Precursor | Product | Collision | |
|---|---|---|---|---|
| Compound | Ion* | Ion* | Energy | CXP |
| 2-Ketobutyric Acid | 101.1 | 56.9 | −12 | −23 |
| 2-Ketobutyric Acid | 107.1 | 60.9 | −12 | −23 |
| ¹³C₄ 3,3-D₂ | | | | |

*Energies, in volts, for the MS/MS analysis

The second period was configured in positive ionization to determine threonine and corresponding internal standard. The conditions on the mass spectrometer were: DP 30, CUR 30, GS1 60, GS2 60, IS 3500, CAD 12, TEMP 500 C. The following transitions were used for the multiple reaction monitoring (MRM):

| | Precursor | Product | Collision | |
|---|---|---|---|---|
| Compound | Ion* | Ion* | Energy | CXP |
| Threonine | 120.1 | 57.0 | 17 | 15 |
| L-Threonine | 125.1 | 60.1 | 17 | 15 |
| U¹³C₄ UD₅ ¹⁵N | | | | |

*Energies, in volts, for the MS/MS analysis

### Example 11

### Production of Propanoic Acid in Engineered Escherichia coli

This example demonstrates that increasing carbon flow through a pathway utilizing threonine increases propionyl-CoA production in host cells which can then be converted to propanoic acid. An Escherichia coli strain was established to overexpress Escherichia coli threonine deaminase (SEQ ID NO: 15), Lactococcus lactis branched-chain 2-ketoacid decarboxylase (KdcA) set out in SEQ ID NO: 33), Salmonella enterica Coenzyme-A acylating propionaldehyde dehydrogenase (PduP) set out in SEQ ID NO: 35, Arabidopsis thaliana acryloyl-CoA oxidase set out in amino acid SEQ ID NO: 1, and the Escherichia coli thioesterase II (TesB), set out in amino acid SEQ ID NO: 1.

In this example, threonine deaminase (SEQ ID NO: 56) promotes the conversion of threonine to 2-ketobutyrate. The Lactococcus lactis branched-chain 2-ketoacid decarboxylase (KdcA) set out in SEQ ID NO: 33) and a Salmonella enterica Coenzyme-A acylating propionaldehyde dehydrogenase (PduP) set out in SEQ ID NO: 35 catalyzes a reaction to convert 2-ketobutyrate to propionyl-CoA. The Arabidopsis thaliana acryl-CoA oxidase catalyzes a reaction to convert propionyl-CoA to acryloyl-CoA. The Escherichia coli thioesterase II (TesB), set out in amino acid SEQ ID NO: 1 catalyzes a reaction to convert acryloyl-CoA to acrylate or propionyl-CoA to propionate.

Vector Constructs

An Escherichia coli expression vector was constructed for overexpression of a recombinant Escherichia coli threonine dehydratase (TdcB). The Escherichia coli tdcB was PCR amplified from the vector pTrcHisA Ec tdcB (SEQ ID NOs: 15 and 16) using the following primers:

Ec tdcB-BB fwd [5′→3′]:
(SEQ ID NO: 87)
TCGAATTCGCGGCCGCTTCTAGAAGGAGATATACATATGGCTCATATTAC
ATACGATCTGCCG;
and
Ec tdcB-BB rev [5′→3′]:
(SEQ ID NO: 88)
AGCTGCAGCGGCCGCTACTAGTATTAGGCGTCAACGAAACCGGTG.

PCR was performed on samples having 30 ng of pTrcHisA Ec tdcB plasmid DNA, 1 μl of a 10 μM stock of each primer, 50 μl of Pfu Ultra II Hotstart 2× master mix (Agilent Technologies, Santa Clara, Calif.), and 47 μl of water. PCR conditions were as follows: the samples were initially incubated at 95° C. for two minutes, followed by thirty cycles at 95° C. for 20 seconds (strand separation), 58° C. for 20 seconds (primer annealing), and 72° C. primer extension for 90 seconds. There was a three minute incubation at 72° C., and the samples were held at 10° C.

The PCR product was purified using a QIAquick® PCR Purification Kit (Qiagen), double digested with restriction enzymes Xba I and Pst I, and ligated (Fast-Link Epicentre Biotechnologies, Madison, Wis.) with SpeI/IPstI-digested pET30a-BB At ACO vector (SEQ ID NO: 1 and 2). The ligation mix was used to transform OneShot Top 10™ Escherichia coli cells (Invitrogen, Carlsbad, Calif.). Individual vials of cells were thawed on ice and gently mixed with 2 μl of ligation mix. The vials were incubated on ice for 30 minutes. The vials were briefly incubated at 42° C. for 30 seconds and quickly replaced back on ice for an additional 2 minutes. 250 μl of 37° C. SOC medium was added and the vials were secured horizontally on a shaking incubator platform and incubated for 1 hour at 37° C., 225 rpm. Aliquotes of 20 μl and 200 μl cells were plated onto selective LB agar (50 μg/ml kanamycin). Single colony isolates were isolated, cultured in 5 ml of selective LB broth and the recombinant plasmid was isolated using a Qiagen Plasmid Mini Kit and characterized by gel electrophoresis of restriction digests with AflIII. DNA sequencing confirmed that the tdcB insert had been cloned and that the insert encoded the published amino acid sequence (Genbank number U00096.2) (SEQ ID NOs: 15 and 16). The resulting plasmid was designated pET30a-BB At ACO_Ec TdcB.

An Escherichia coli expression vector was constructed for overexpression of a recombinant Salmonella enterica Coenzyme-A acylating propionaldehyde dehydrogenase (PduP) and Lactococcus lactis branched-chain 2-ketoacid decarboxylase (KdcA). The codon optimized Lactococcus lactis branched-chain 2-ketoacid decarboxylase (kdcA) from pET30a-BB Ll KDCA was cloned into pET30a-BB Se PDUP (Example 3) by double digestion of pET30a-BB Ll KDCA with restriction enzymes Xba I and Pst I. The Ll KDCA fragment was band isolated, purified using a QIAquick Gel Extraction Kit (Qiagen, Carlsbad, Calif.) and ligated (Fast-Link Epicentre Biotechnologies, Madison, Wis.) with SpeI/PstI-digested pET30a-BB Se PDUP vector. The ligation mix was used to transform OneShot Top 10™ Escherichia coli cells (Invitrogen, Carlsbad, Calif.). Individual vials of cells were thawed on ice and gently mixed with 2 μl of ligation mix. The vials were incubated on ice for 30 minutes. The vials were briefly incubated at 42° C. for 30 seconds and quickly replaced back on ice for an additional 2 minutes. 250 μl of 37° C. SOC medium was added and the vials were secured horizontally on a shaking incubator platform and incubated for 1 hour at 37° C., 225 rpm. Aliquotes of 20 μl and 200 μl cells were plated onto selective LB agar (50 μg/ml kanamycin). Single colony isolates were isolated, cultured in 5 ml of selective LB broth and the recombinant plasmid was isolated using a Qiagen Plasmid Mini Kit and characterized by gel electrophoresis of restriction digests with AflIII. The resulting plasmid was designated pET30a-BB Se PDUP_Ll KDCA.

To facilitate cotransformation with pET30a-BB At ACO_Ec TdcB_Ec TesB the codon optimized Salmonella enterica Coenzyme-A acylating propionaldehyde dehydrogenase (PduP) and L. lactis Branched-chain 2-ketoacid decarboxylase (KdcA) gene pair was subcloned from pET30a-BB Se PDUP_Ll KDCA into the pCDFDuet-1 vector (Novagen [EMD Chemicals, Gibbstown, N.J.] #71340-3) by double digestion of pET30a-BB Se PDUP_Ll KDCA with restriction enzymes EcoRI and Pst I. The Se PDUP_Ll KDCA fragment was band isolated, purified using a QIAquick Gel Extraction Kit (Qiagen, Carlsbad, Calif.) and ligated (Fast-Link Epicentre Biotechnologies, Madison, Wis.) with EcoRI/PstI-digested pCDFDuet-1. The ligation mix was used to transform OneShot Top 10™ Escherichia coli cells (Invitrogen, Carlsbad, Calif.). Individual vials of cells were thawed on ice and gently mixed with 2 μl of ligation mix. The vials were incubated on ice for 30 minutes. The vials were briefly incubated at 42° C. for 30 seconds and quickly replaced back on ice for an additional 2 minutes. 250 μl of 37° C. SOC medium was added and the vials were secured horizontally on a shaking incubator platform and incubated for 1 hour at 37° C., 225 rpm. Aliquots of 20 μl and 200 μl cells were plated onto selective LB agar (50 μg/ml spectinomycin). Single colony isolates were isolated, cultured in 5 ml of selective LB broth and the recombinant plasmid was isolated using a Qiagen Plasmid Mini Kit and characterized by gel electrophoresis of restriction digests with AflIII. The resulting plasmid was designated pCDFDuet-1 Se PDUP_Ll KDCA.

Co-Transformation of Escherichia coli

The recombinant plasmids and empty parent vectors were used to co-transform chemically competent BL21 (DE3) pLysS Escherichia coli cells (Invitrogen, Carlsbad, Calif.) in the following combinations:

pET30a-BB AtACO_Ec TdcB_Ec TesB and pCDFDuet-1 Se PDUP_Ll KDCA

pET30a-BB AtACO_Ec TdcB and pCDFDuet-1 Se PDUP_Ll KDCA

pET30a-BB and pCDFDuet-1

Individual vials of cells were thawed on ice and gently mixed with 50 μs of plasmid DNA. The vials were incubated on ice for 30 minutes. The vials were briefly incubated at 42° C. for 30 seconds and quickly replaced back on ice for an additional 2 minutes. 250 μl of 37° C. SOC medium was added and the vials were secured horizontally on a shaking incubator platform and incubated for 1 hour at 37° C., 225 rpm. Aliquotes of 20 μl and 200 μl cells were plated onto selective LB agar (50 μg/ml kanamycin; 50 μg/ml spectinomycin; 34 μg/ml chloramphenicol) plates to select for cells carrying the recombinant pET30a-BB, pCDFDuet-1 and pLysS plasmids respectively and incubated overnight at 37° C. Single colony isolates were isolated, cultured in 5 ml of selective LB broth and the recombinant plasmids were isolated using a QIAPrep® Spin Miniprep Kit (Qiagen, Valencia, Calif.) and characterized by gel electrophoresis of restriction digests with AvaI.

Strain Culture

Overnight cultures of the co-transformed BL21 (DE3) pLysS strains (10 ml of minimal M9 media; 34 μg/ml chloramphenicol; 50 μg/ml kanamycin and 50 μg/ml spectinomycin) in 50 ml conical tubes were inoculated from single colony forming units from minimal M9 agar plates. Cultures were incubated overnight at 37° C. with 250 rpm shaking. Fresh cultures (30 ml of minimal M9 media; 34 μg/ml chloramphenicol; 50 μg/ml kanamycin and 50 μg/ml spectinomycin) in 250 ml Erlenmeyer flasks were inoculated from the overnight cultures at an optical density at 600 nm (OD₆₀₀) of ˜0.01. The second cultures were incubated at 37° C. with 250 rpm shaking overnight. Two sets of test cultures (50 ml of minimal M9 media; 34 μg/ml chloramphenicol; 50 μg/ml kanamycin and 50 μg/ml spectinomycin) in 500 ml Erlenmeyer flasks were inoculated from the second overnight cultures at an OD₆₀₀ of ˜0.2. One set of these cultures was further supplemented with 1 g/L L-threonine (Sigma-Adrich). All cultures were incubated at 25° C. with 250 rpm shaking and optical density monitored until OD₆₀₀ of ˜0.4. All cultures were then supplemented with 100×BME vitamins (Sigma-Aldrich) at a 10× final concentration and plasmid recombinant gene protein expression was then induced by addition of 50 μL of 1M IPTG (Teknova, Hollister, Calif.; 1 mM final concentration). Cultures were further incubated for 18 hours at 25° C. with 250 rpm shaking before the cells were processed for analysis and stored at −80° C.

| | 1X Base Recipe | |
|---|---|---|
| Minimal M9 Media Component | | |
| Na₂HPO₄ | 6 | g/L |
| KH₂PO₄ | 3 | g/L |
| NaCl | 0.5 | g/L |
| NH₄Cl | 1 | g/L |
| CaCl₂ * 2H₂O | 0.1 | mM |
| MgSO₄ | 1 | mM |
| Dextrose | 80 | mM |
| Thiamine | 1 | mg/L |
| Chloramphenicol | 34 | μg/mL |
| Kanamycin | 50 | μg/mL |
| Spectinomycin | 50 | μg/mL |
| 100X BME Vitamins (added as 10X; | | |
| Sigma-Aldrich, St. Louis, MO) | | |
| D-Biotin (0.1 g/L) | 10 | mg/L |
| Choline Chloride (0.1 g/L) | 10 | mg/L |
| Folic Acid (0.1 g/L) | 10 | mg/L |
| myo-Inositol (0.2 g/L) | 20 | mg/L |
| Niacinamide (0.1 g/L) | 10 | mg/L |
| p-Amino Benzoic Acid (0.1 g/L) | 10 | mg/L |
| D-Pantothenic Acid • ½Ca (0.1 g/L) | 10 | mg/L |
| Pyridoxal · HCl (0.1 g/L) | 10 | mg/L |
| Riboflavin (0.01 g/L) | 1 | mg/L |
| Thiamine • HCl (0.1 g/L) | 10 | mg/L |
| NaCl (8.5 g/L) | 0.85 | g/L |

Production of Propionic Acid by Engineered Escherichia coli

The data shows that the presence of intermediates and propionic acid in the threonine to propionic acid pathway are dependent upon the expression of the genes. Endogenous threonine likely supports production when no exogenous threonine was added to the culture medium.

When threonine is added, an increase in 2-ketobutyrate and propionic acid was observed.

| | | | | Propionic |
|---|---|---|---|---|
| Expressed | Threonine | 2-Ketobutyrate | Propionyl- | Acid in |
| Heterologous | Addition | in Broth | CoA | Broth |
| Genes | (g/L) | (μg/ml) | (ng/mL) | (μg/ml) |
| tdcB, kdcA, | 0 | <0.25 | 204 | 16 |
| pduP, ACO, | | | | |
| tesB | | | | |
| tdcB, kdcA, | 0 | 5.1 | 415 | 7.5 |
| pduP, ACO | | | | |
| None | 0 | <0.25 | 9.3 | 0.9 |
| tdcB, kdcA, | 1 | 14.7 | 317 | 35 |
| pduP, ACO, | | | | |
| tesB | | | | |
| tdcB, kdcA, | 1 | 31 | 425 | 17 |
| pduP, ACO | | | | |
| None | 1 | 1.0 | 8.8 | 2.0 |

### Example 12

### Engineering the TCA cycle in E. coli for the high yield production of propionic acid

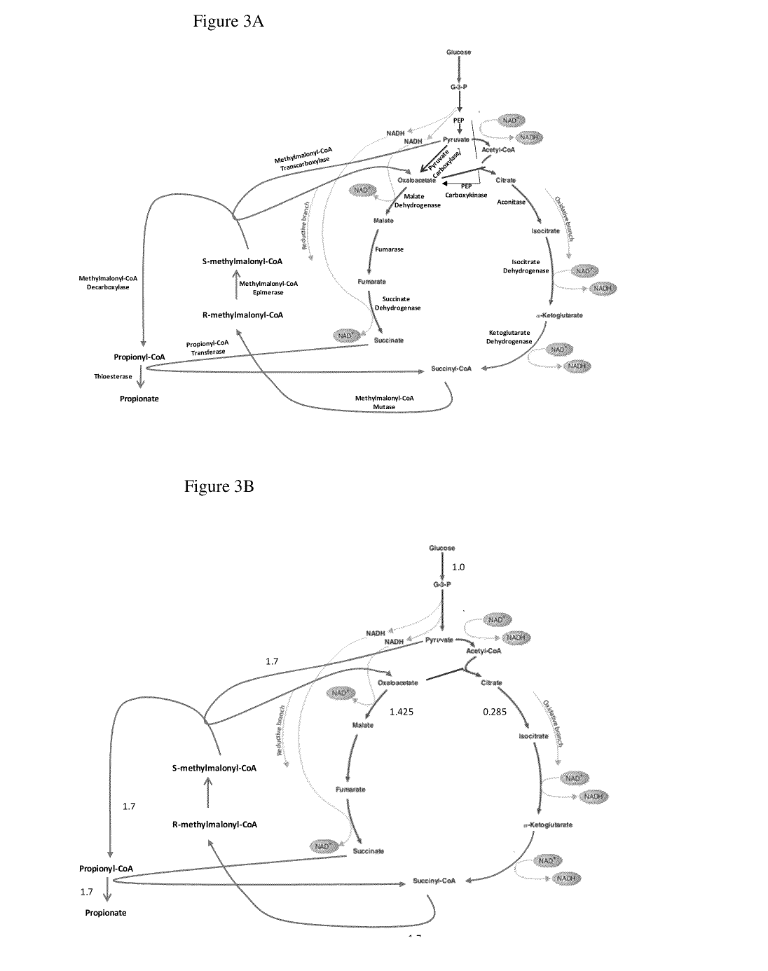

To improve the yield of propionic acid and minimize the production of acetic acid, the pathway (illustrated in FIG. 3) that combines the oxidative branch and reductive branch of the TCA cycle maximizes the production of succinyl-CoA and eventually the propionate. The oxidative branch of the TCA cycle will produce additional reduction power needed to reduce pyruvate to priopionic acid and resulted in a theoretical maximum mass yield of 70%. Each of the steps proposed in the pathway should not have thermodynamic barriers as the shown in the table.

| | | ΔG°¹ |
|---|---|---|
| Reaction | Enzyme | (kJ · mol⁻¹) |
| 1 | Citrate synthase | −31.5 |
| 2 | Aconitase | ~5 |
| 3 | Isocitrate dehydrogenase | −21 |
| 4 | α-Ketoglutarate | −33 |
| | dehydrogenase | |
| | multienzyme complex | |
| 5 | Succinyl-CoA synthetase | −2.1 |
| 6 | Succinate dehydrogenase | +6 |
| 7 | Fumarase | −3.4 |
| 8 | Malate dehydrogenase | +29.7 |

Running reaction 8 in reverse is thermodynamically favored. The reductive branch of the TCA cycle is already used in the Wood-Werkman Cycle, the oxidative branch is normal part of the TCA cycle. No new enzymes need to be discovered to achieve the pathway.

The fluxes to the oxidative branch and reductive branch are balanced as illustrated in FIG. 3B to achieve the maximum yield. To achieve this balance, the activity of key enzymes is tuned by adjusting expression or introducing mutations that reduce or enhance activity. The activities of enzymes at two critical branch points, malate dehydrogenase and citrate synthase, and methylmalonyl-CoA transcarboxylase and pyruvate dehydrogenase, are adjusted. Other critical enzymes at branch points are adjusted, including phosphoenolpyruvate carboxykinase and pyruvate kinase, and pyruvate dehydrogenase and pyruvate carboxylase. Because any step in the pathway (and even those not in the pathway) may affect the flux balance, other enzymes in the pathway and their regulators are tuned as well. A methylmalonyl-CoA transcarboxylase (Seq ID NOs: 93, 95, 97, 99, 101, 103 and 105) is cloned and expressed. The methylmalonyl-CoA transcarboxylase is a complex enzyme. If it cannot easily be expressed, its activity is replaced using a methyl malonyl-CoA decarboxylase (Seq ID NO: 43) and the other a phosphoenolpyruvate (PEP) carboxylase (or PEP carboxykinase (Seq ID NO: 147). A PEP carboxykinase has the advantage to generate a ATP in the process. Alternatively, pyruvate kinase (Seq ID NOs: 149 and 151) and pyruvate carboxylase (SEQ ID NO: 107) are expressed to generate oxaloacetate.

Production under anaerobic conditions may not generate enough energy. In the original Wood-Werkman Cycle acetic acid production step, the high energy thioester bond of acetyl-CoA is recovered as ATP. The proposed new pathway eliminates the acetate production, and hence reduced the amount of ATP produced. However, since none of the enzymes appear to be oxygen sensitive, the pathway may be run under microaerobic conditions to generate the small amount of energy that may be required.

To avoid succinic acid accumulation, the activity of B12 dependent methylmalonyl-CoA transcarboxylase (Seq ID NOs: 39 and 41) that converts succinyl-CoA to methylmalonyl-CoA is improved by overexpressing the enzyme, or is replaced with more active enzymes.

The foregoing description is given for clearness of understanding only, and no unnecessary limitations should be understood therefrom, as modifications within the scope of the invention may be apparent to those having ordinary skill in the art.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. A method for producing propionic acid comprising the steps of:
a. converting threonine to 2-ketobutyrate;
b. converting 2-ketobutyrate to propionyl-CoA; and
c. converting propionyl-CoA to propionic acid.

2. The method of claim 1 wherein a microorganism expresses at least one heterologous gene encoding an enzyme, wherein the enzyme is selected from the group consisting of a dehydratase, a deaminase, a dehydrogenase, a ketoacid decarboxylase, a lyase, and a thioesterase, acyltransferase/kinase or CoA transferase.

3. The method of claim 1 wherein at least one isolated enzyme selected from the group consisting of a dehydratase, a deaminase, a dehydrogenase, a ketoacid decarboxylase, a lyase, a thioesterase, an acyltransferase/kinase and CoA transferase is used.

4. A method for producing propionic acid comprising the steps of:
a. converting threonine to 2-ketobutyrate;
b. converting 2-ketobutyrate to propionaldehyde; and
c. converting propionaldehyde to propionic acid.

5. The method of claim 4 wherein a microorganism expresses at least one heterologous gene encoding an enzyme, wherein the enzyme is selected from the group consisting of a dehydratase or deaminase, a ketoacid decarboxylase, and a dehydrogenase.

6. The method of claim 4 wherein at least one isolated enzyme selected from the group consisting of a dehydratase, a deaminase, a ketoacid decarboxylase and a dehydrogenase is used.

7. The propionic acid produced by the method of claim 1.

8. The propionic acid produced by the method of claim 4.

9. A method for producing propionic acid comprising the steps of:
a. converting pyruvate to oxaloacetate;
b. converting oxaloacetate to malate;
c. converting malate to fumarate;
d. converting fumarate to succinate;
e. converting succinate to succinyl-CoA; and
f. converting succinyl-CoA to propionic acid;

10. The method of claim 9 wherein oxaloacetate is also reacted with acetyl-CoA to form citrate, citrate is converted to aconitate, aconitate is converted to isocitrate, isocitrate is converted to α-ketoglutarate, α-ketoglutarate is converted to succinyl-CoA, and wherein succinyl-CoA is converted to propionic acid.

11. The method of claim 10 wherein a microorganism expresses at least one heterologous gene encoding an enzyme, wherein the enzyme is selected from the group consisting of a transcarboxylase, a carboxylase, a dehydrogenase, a hydratase, a dehydrogenase, an acyl-CoA transferase, an acyl-CoA synthetase, a mutase, an epimerase, a decarboxylase, a synthase, an aconitase, a isocitrate dehydrogenase, and an α-ketoglutarate dehydrogenase.

12. A method for producing propionic acid comprising the steps of:
a. converting phosphoenolpyruvate to oxaloacetate;
b. converting oxaloacetate to malate;
c. converting malate to fumarate;
d. converting fumarate to succinate;
e. converting succinate to succinyl-CoA;
f. converting succinyl-CoA to R-methymalonyl-CoA
g. converting R-methylmalonyl-CoA to S-methylmalonyl-CoA, and
h. converting S-methylmalonyl-CoA to propionic acid.

13. The method of claim 12 wherein oxaloacetate is also reacted with acetyl-CoA to form citrate, citrate is converted to aconitate, aconitate is converted to isocitrate, isocitrate is converted to α-ketoglutarate, α-ketoglutarate is converted to succinyl-CoA, succinyl-CoA is converted to R-methylmalonyl-CoA, R-methylmalonyl-CoA is converted to S-methylmalonyl-CoA, and wherein S-methylmalonyl-CoA is converted to propionic acid.

14. The method of claim 12 wherein a microorganism expresses at least one heterologous gene encoding an enzyme, wherein the enzyme is selected from the group consisting of a carboxykinase, a dehydrogenase, a hydratase, a dehydrogenase, an acyl-CoA transferase, an acyl-CoA synthetase, a mutase, an epimerase and a decarboxylase, a synthase, an aconitase, a isocitrate dehydrogenase, and an α-ketoglutarate dehydrogenase.

15. The propionic acid produced by the method of claim 9.

16. The propionic acid produced by the method of claim 12.

17. A method for producing propionic acid wherein pyruvate is converted to propionic acid.

18. The method of claim 17 comprising the steps of:
a. converting pyruvate to citramalate;
b. converting citramalate to citraconate;
c. converting citraconate to β-methyl-D-malate;
d. converting β-methyl-D-malate to 2-ketobutyrate;
e. converting 2-ketobutyrate to propionyl-CoA; and
f. converting propionyl-CoA to propionic acid.

19. The method of claim 18 wherein a microorganism expresses at least one heterologous gene encoding an enzyme, wherein the enzyme is selected from the group consisting of a synthase, a hydrolase, a dehydratase or isomerase, a dehydrogenase, a ketoacid dehydrogenase, a ketoacid decarboxylase, a lyase, a thioesterase, phosphate acyltransferase/kinase or acyl-CoA transferase.

20. The method of claim 18 wherein at least one isolated enzyme selected from the group consisting of a synthase, a hydrolase, a dehydratase or isomerase, a dehydrogenase, a ketoacid dehydrogenase, a ketoacid decarboxylase, a lyase, and a thioesterase, phosphate acyltransferase/kinase or acyl-CoA transferase is used.

21. The method of claim 17 comprising the steps of:
a. converting pyruvate to citramalate;
b. converting citramalate to citraconate;
c. converting citraconate to β-methyl-D-malate;
d. converting β-methyl-D-malate to 2-ketobutyrate;
e. converting 2-ketobutyrate to propionaldehyde; and
f. converting propionaldehyde to propionic acid.

22. The method of claim 21 wherein a microorganism expresses at least one heterologous gene encoding an enzyme, wherein the enzyme is selected from the group consisting of a synthase, a hydrolase, a dehydratase or isomerase, a dehydrogenase, a ketoacid decarboxylase, and a dehydrogenase.

23. The method of claim 21 wherein at least one isolated enzyme selected from the group consisting of a synthase, a hydrolase, a dehydratase or isomerase, a dehydrogenase, a ketoacid decarboxylase, and a dehydrogenase is used.

24. The propionic acid produced by the method of claim 17.


SEQUENCE LISTING (176 sequences)

SEQ ID NO: 1 - protein, 270 aa, E. coli
MSQALKNLLTLLNLEKIEEGLFRGQSEDLGLRQVFGGQVVGQALYAAKETVPEERLVHSFHSYFLRPGDSKKPIIYDVETNGKPIFYMTASFQAPEAGFEHQKTMPSAPAPDGLPSETQIAQSLAHLLPPVLKDKFICDRPLEVRPVEFHNPLKGHVAEPHRQVWIRANGSVPDDLRVHQYLLGYASDLNFLPVALQPHGIGFLEPGIQIATIDHSMWFHRPFNLNEWLLYSVESTSASSARGFVRGEFYTQDGVLVASTVQEGVMRNHN

SEQ ID NO: 2 - DNA, 888 bp, E. coli
ggatccatgtctagaatgagccaagccctgaaaaacctgctgacgctgctgaatctggaaaaaatcgaagaaggcctgttccgtggtcaatctgaagacctgggcctgcgtcaggtgtttggcggtcaggtggttggtcaagcgctgtatgcggccaaagaaaccgttccggaagaacgtctggtccatagctttcactcttatttcctgcgcccgggcgatagcaaaaaaccgattatctacgatgtggaaaccctgcgcgacggcaacagtttttccgcccgtcgcgttgcagctattcagaatggtaaaccgatcttttacatgacggcatcattccaggcaccggaagctggctttgaacatcaaaaaaccatgccgagcgccccggcaccggatggtctgccgagtgaaacgcagattgcacaatccctggctcatctgctgccgccggtcctgaaagataaatttatctgtgaccgtccgctggaagtccgtccggtggaatttcacaacccgctgaaaggccatgtcgcagaaccgcaccgtcaagtgtggattcgcgctaatggcagcgtgccggatgacctgcgtgttcatcaatatctgctgggttacgcgtctgatctgaactttctgccggttgccctgcaaccgcacggcattggtttcctggaaccgggtattcaaatcgccacgatcgaccattcaatgtggtttcaccgcccgttcaacctgaatgaatggctgctgtattccgttgaatcaaccagcgcgagcagcgcccgtggctttgtccgtggtgaattttacacgcaagatggtgtcctggtggcgtctaccgttcaagaaggcgttatgcgtaatcacaactaagagctcaagctt

SEQ ID NO: 3 - protein, 524 aa, Clostridium propionicum
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTADSGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

SEQ ID NO: 4 - DNA, 1602 bp, Clostridium propionicum
ggatccatgtctagaatgcgcaaagtcccgattattacggcagatgaagcggctaaactgattaaagacggcgatacggtcaccaccagcggtttcgttggcaacgcaattccggaagctctggatcgtgcggttgaaaaacgctttctggaaaccggcgaaccgaaaaacatcacgtatgtctactgcggcagtcagggtaatcgtgatggccgcggtgccgaacatttcgcacacgaaggcctgctgaaacgttatattgctggtcattgggccaccgtcccggcactgggtaaaatggcaatggaaaacaaaatggaagcgtataatgtgtcacagggcgcgctgtgtcacctgtttcgtgatattgcctcgcacaaaccgggtgtctttaccaaagtgggcattggtacgtttatcgacccgcgcaacggcggtggcaaagtgaatgatattaccaaagaagacatcgtcgaactggtggaaattaaaggccaggaatacctgttttatccggcgttcccgattcatgttgccctgatccgcggcacctatgccgatgaatctggtaacattacgtttgaaaaagaagtggcaccgctggaaggcaccagcgtgtgccaggcagtcaaaaattctggtggcatcgtggttgtccaagttgaacgtgtggttaaagcgggcaccctggacccgcgccacgttaaagtcccgggtatttatgtggactacgtcgtggttgctgatccggaagaccatcagcaaagtctggattgtgaatatgatccggcactgtccggtgaacaccgtcgcccggaagttgtgggtgaaccgctgccgctgagtgctaaaaaagttattggccgtcgcggtgcgatcgaactggaaaaagatgtggccgttaacctgggcgtgggtgcaccggaatacgttgcgtccgtcgccgatgaagaaggcattgttgactttatgaccctgacggcagatagcggtgctattggcggcgtgccggcgggcggcgttcgttttggcgcgtcttataatgcggatgccctgatcgaccagggttaccaattcgattattacgacggtggcggtctggatctgtgctatctgggcctggcggaatgtgacgaaaagggtaacattaatgtgtcacgttttggtccgcgtattgcgggttgtggtggtttcattaacatcacccagaatacgccgaaagtctttttctgtggcacctttacggcaggcggtctgaaagtgaaaattgaagatggcaaagtgattatcgttcaggaaggtaaacagaaaaaattcctgaaagcggttgaacaaatcaccttcaacggtgatgtcgcactggctaataaacagcaagtgacctatatcacggaacgttgcgtttttctgctgaaagaagatggcctgcacctgtcggaaattgcgccgggtattgatctgcaaacccaaattctggatgtgatggacttcgccccgattatcgatcgcgacgcaaatggccagatcaaactgatggatgcggcactgtttgcggaaggtctgatgggtctgaaagaaatgaaatcgtaagagctcaagctt

SEQ ID NO: 5 - protein, 517 aa, Megasphaera elsdenii
MRKVEIITAEQAAQLVKDNDTITSIGFVSSAHPEALTKALEKRFLDTNTPQNLTYIYAGSQGKRDGRAAEHLAHTGLLKRAIIGHWQTVPAIGKLAVENKIEAYNFSQGTLVHWFRALAGHKLGVFTDIGLETFLDPRQLGGKLNDVTKEDLVKLIEVDGHEQLFYPTFPVNVAFLRGTYADESGNITMDEEIGPFESTSVAQAVHNCGGKVVVQVKDVVAHGSLDPRMVKIPGIYVDYVVVAAPEDHQQTYDCEYDPSLSGEHRAPEGAADAALPMSAKKIIGRRGALELTENAVVNLGVGAPEYVASVAGEEGIADTITLTVDGGAIGGVPQGGARFGSSRNADAIIDHTYQFDFYDGGGLDIAYLGLAQCDGSGNINVSKFGTNVAGCGGFPNISQQTPNVYFCGTFTAGGLKIAVEDGKVKILQEGKAKKFIKAVDQITFNGSYAARNGKHVLYITERCVFELTKEGLKLIEVAPGIDIEKDILAHMDFKPIIDNPKLMDARLFQDGPMGLKR

SEQ ID NO: 6 - DNA, 1581 bp, Megasphaera elsdenii
ggatccatgtctagaatgcgtaaagttgaaattattaccgcagaacaggcagcacagctggttaaagataatgataccattaccagcattggctttgttagcagcgcacatccggaagcactgaccaaagcactggaaaaacgttttctggataccaatacaccgcagaatctgacctatatttatgcaggtagccagggtaaacgtgatggtcgtgcagcagaacatctggcacatacaggtctgctgaaacgtgcaattattggtcattggcagaccgttccggcaattggtaaactggcagtggaaaataaaattgaagcctataattttagccagggcaccctggttcattggtttcgtgcactggcaggtcataaactgggtgtttttaccgatattggcctggaaacctttctggacccgcgtcagctgggtggtaaactgaatgatgttaccaaagaggatctggttaaactgattgaagtggatggtcatgaacagctgttttatccgacctttccggttaatgttgcatttctgcgtggcacctatgcagatgaaagcggtaatattacaatggatgaagaaattggtccgtttgaaagcaccagcgttgcacaggcagttcataattgtggtggtaaagttgtggttcaggttaaagatgttgttgcacatggtagcctggacccgcgtatggttaaaattccgggtatttatgtggattatgttgttgttgcagcaccggaagatcatcagcagacctatgattgtgaatatgatccgagcctgagcggtgaacatcgtgcaccggaaggtgcagcagatgcagcactgccgatgagcgcaaaaaaaattattggtcgtcgtggtgcactggaactgaccgaaaatgcagttgttaatctgggtgttggtgcaccggaatatgttgcaagcgttgcgggtgaagaaggtattgcagataccattacactgaccgttgatggtggtgcaattggtggtgttccgcagggtggtgcacgttttggtagcagccgtaatgcagatgccattattgatcatacctatcagtttgatttttatgatggtggtggtctggatattgcatatctgggtctggcacagtgtgatggtagtggtaatattaatgtgagcaaatttggcaccaatgttgcaggttgtggtggttttccgaatattagccagcagaccccgaatgtttatttttgtggcacctttaccgcaggcggtctgaaaattgcagttgaagatggcaaagtgaaaattctgcaagaaggcaaagccaaaaaatttattaaagccgtggatcagattacctttaatggtagctatgcagcccgtaatggtaaacatgttctgtatattaccgaacgctgcgtttttgaactgacaaaagaaggtctgaaactgatcgaagttgcaccgggtattgatattgaaaaagatattctggcccacatggattttaaaccgattattgataatccgaaactgatggatgcccgtctgtttcaggatggtccgatgggtctgaaacgttaagagctcaagctt

SEQ ID NO: 7 - protein, 714 aa, Escherichia coli
VSRIIMLIPTGTSVGLTSVSLGVIRAMERKGVRLSVFKPIAQPRTGGDAPDQTTTIVRANSSTTTAAEPLKMSYVEGLLSSNQKDVLMEEIVANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSSKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIESLTATSERSRRLSPPAFRYQLTELARKAGKRIVLPEGDEPRTVKAAAICAERGIATCVLLGNPAEINRVAASQGVELGAGIEIVDPEVVRESYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 8 - DNA, 2145 bp, Escherichia coli
gtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcactgacaccccggaacagctgaaagagcgtatcgaactgacccgcaacagcttcggcggtgccaaaaacaccaacatcaccggcgttatcgttaacaaactgaacgcaccggttgatgaacagggtcgtactcgcccggatctgtccgagattttcgacgactcttccaaagctaaagtaaacaatgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgtcctgacgtgctggtggccgcttgcctggcagccatgaacggcgtagaaatcggtgccctgctgctgactggcggttacgaaatggacgcgcgcatttctaaactgtgcgaacgtgctttcgctaccggcctgccggtatttatggtgaacaccaacacctggcagacctctctgagcctgcagagcttcaacctggaagttccggttgacgatcacgaacgtatcgagaaagttcaggaatacgttgctaactacatcaacgctgactggatcgaatctctgactgccacttctgagcgcagccgtcgtctgtctccgcctgcgttccgttatcagctgactgaacttgcgcgcaaagcgggcaaacgtatcgtactgccggaaggtgacgaaccgcgtaccgttaaagcagccgctatctgtgctgaacgtggtatcgcaacttgcgtactgctgggtaatccggcagagatcaaccgtgttgcagcgtctcagggtgtagaactgggtgcagggattgaaatcgttgatccagaagtggttcgcgaaagctatgttggtcgtctggtcgaactgcgtaagaacaaaggcatgaccgaaaccgttgcccgcgaacagctggaagacaacgtggtgctcggtacgctgatgctggaacaggatgaagttgatggtctggtttccggtgctgttcacactaccgcaaacaccatccgtccgccgctgcagctgatcaaaactgcaccgggcagctccctggtatcttccgtgttcttcatgctgctgccggaacaggtttacgtttacggtgactgtgcgatcaacccggatccgaccgctgaacagctggcagaaatcgcgattcagtccgctgattccgctgcggccttcggtatcgaaccgcgcgttgctatgctctcctactccaccggtacttctggtgcaggtagcgacgtagaaaaagttcgcgaagcaactcgtctggcgcaggaaaaacgtcctgacctgatgatcgacggtccgctgcagtacgacgctgcggtaatggctgacgttgcgaaatccaaagcgccgaactctccggttgcaggtcgcgctaccgtgttcatcttcccggatctgaacaccggtaacaccacctacaaagcggtacagcgttctgccgacctgatctccatcgggccgatgctgcagggtatgcgcaagccggttaacgacctgtcccgtggcgcactggttgacgatatcgtctacaccatcgcgctgactgcgattcagtctgcacagcagcagtaa

SEQ ID NO: 9 - protein, 450 aa, Escherichia coli
MNEFPVVLVINCGSSSIKFSVLDASDCEVLMSGIADGINSENAFLSVNGGEPAPLAHHSYEGALKAIAFELEKRNLNDSVALIGHRIAHGGSIFTESAIITDEVIDNIRRVSPLAPLHNYANLSGIESAQQLFPGVTQVAVFDTSFHQTMAPEAYLYGLPWKYYEELGVRRYGFHGTSHRYVSQRAHSLLNLAEDDSGLVVAHLGNGASICAVRNGQSVDTSMGMTPLEGLMMGTRSGDVDFGAMSWVASQTNQSLGDLERVVNKESGLLGISGLSSDLRVLEKAWHEGHERAQLAIKTFVHRIARHIAGHAASLRRLDGIIFTGGIGENSSLIRRLVMEHLAVLGLEIDTELVMEHLAVLGLEIDTEMNNRSNSCGERIVSSEMNNRSNSCGERIVSSENARVICAVIPTNEEKMNARVICAVIPTNEEKMIALDAIHLGKVNAPAEFA

SEQ ID NO: 10 - DNA, 1209 bp, Escherichia coli
atgaatgaatttccggttgttttggttattaactgtggttcgtcttcgattaagttttccgtgctcgatgccagcgactgtgaagtattaatgtcaggtattgccgacggtattaactcggaaaatgcattcttatccgtaaatgggggagagccagcaccgctggctcaccacagctacgaaggtgcattgaaggcaattgcatttgaactggaaaaacggaatttaaatgacagtgtggccttaattggccaccgcatcgctcacggcggcagtatttttaccgagtccgccattattaccgatgaagtcattgataatatccgtcgcgtttctccactggcacccctgcataattacgccaatttaagtggtattgaatcggcgcagcaattatttccgggcgtaactcaggtggcggtatttgataccagtttccaccagacgatggctccggaagcttatttatacggcctgccgtggaaatattatgaagagttaggtgtacgccgttatggtttccacggcacgtcgcaccgctatgtttcccagcgcgcacattcgctgctgaatctggcggaagatgactccggcctggttgtggcgcatcttggcaatggcgcgtcaatctgcgcggttcgcaacggtcagagtgttgatacctcaatgggaatgacgccgctggaaggcttgatgatgggtacccgcagtggcgatgtcgactttggtgcgatgtcctgggtcgccagccaaaccaaccagagcctgggtgacctggaacgcgtagtgaataaagagtcgggattattaggtatttccggtctttcttcggatttacgtgttctggaaaaagcctggcatgaaggtcacgaacgcgcgcaactggcaattaaaacctttgttcaccgaattgcccgtcatattgccggacacgcagcttcattacgtcgcctggatggaattatattcaccggcggaataggagagaattcaagcttaattcgtcgtctggtcatggaacatttggctgtattaggcttagagattgatacagaaatgaataatcgctctaactcctgtggtgagcgaattgtttccagtgaaaatgcgcgtgtcatttgtgccgttattccgactaacgaagaaaaaatgattgctttggatgccattcatttaggcaaagttaacgcgcccgcagaatttgcataa

SEQ ID NO: 11 - protein, 524 aa, Clostridium propionicum
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTAESGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

SEQ ID NO: 12 - DNA, 1575 bp, Clostridium propionicum
atgagaaaggttcccattattaccgcagatgaggctgcaaagcttattaaagacggtgatacagttacaacaagtggtttcgttggaaatgcaatccctgaggctcttgatagagctgtagaaaaaagattcttagaaacaggcgaacccaaaaacattacatatgtttattgtggttctcaaggtaacagagacggaagaggtgctgagcactttgctcatgaaggccttttaaaacgttacatcgctggtcactgggctacagttcctgctttgggtaaaatggctatggaaaataaaatggaagcatataatgtatctcagggtgcattgtgtcatttgttccgtgatatagcttctcataagccaggcgtatttacaaaggtaggtatcggtactttcattgaccccagaaatggcggcggtaaagtaaatgatattaccaaagaagatattgttgaattggtagagattaagggtcaggaatatttattctaccctgcttttcctattcatgtagctcttattcgtggtacttacgctgatgaaagcggaaatatcacatttgagaaagaagttgctcctctggaaggaacttcagtatgccaggctgttaaaaacagtggcggtatcgttgtagttcaggttgaaagagtagtaaaagctggtactcttgaccctcgtcatgtaaaagttccaggaatttatgttgactatgttgttgttgctgacccagaagatcatcagcaatctttagattgtgaatatgatcctgcattatcaggcgagcatagaagacctgaagttgttggagaaccacttcctttgagtgcaaagaaagttattggtcgtcgtggtgccattgaattagaaaaagatgttgctgtaaatttaggtgttggtgcgcctgaatatgtagcaagtgttgctgatgaagaaggtatcgttgattttatgactttaactgctgaaagtggtgctattggtggtgttcctgctggtggcgttcgctttggtgcttcttataatgcggatgcattgatcgatcaaggttatcaattcgattactatgatggcggcggcttagacctttgctatttaggcttagctgaatgcgatgaaaaaggcaatatcaacgtttcaagatttggccctcgtatcgctggttgtggtggtttcatcaacattacacagaatacacctaaggtattcttctgtggtactttcacagcaggtggcttaaaggttaaaattgaagatggcaaggttattattgttcaagaaggcaagcagaaaaaattcttgaaagctgttgagcagattacattcaatggtgacgttgcacttgctaataagcaacaagtaacttatattacagaaagatgcgtattccttttgaaggaagatggtttgcacttatctgaaattgcacctggtattgatttgcagacacagattcttgacgttatggattttgcacctattattgacagagatgcaaacggccaaatcaaattgatggacgctgctttgtttgcagaaggcttaatgggtctgaaggaaatgaagtcctga

SEQ ID NO: 13 - protein, 329 aa, Klebsiella pneumonia
MHITYDLPVSIDDILEAKQRLAGKIYKTGMPRSNYFSEHCQGEIFLKFENMQRTGSFKIRGAFNKLCGLTAAEKRKGVVACSAGNHAQGVSLSCAMLGIDGKVVMPKGAPKSKVAATCDYSAEVVLHGDNFNDTLAKASDIVELEGRIFIPPYDDPQVIAGQGTIGLEILEDLYDVDNVIVPIGGGGLIAGIAIAIKSINPTIRIIGVQSENVHGMAASWYAGEITSHRHAGTLADGCDVARPGKLTYEIARQLVDDIVLVSEDDIRQSMVALIQRNKVITEGAGALACAALLSGKLDSYIQNRKTVSLISGGNIDLSRVSQITGFVDA

SEQ ID NO: 14 - DNA, 990 bp, Klebsiella pneumonia
atgcatattacctacgatcttccggtatccattgacgatattctcgaggcgaagcaacgcctggcgggaaaaatatataaaacgggcatgccccgctcgaattattttagcgaacactgccagggggaaatattccttaaattcgaaaatatgcagcgcacgggctcatttaaaattcgcggcgcgtttaataagctctgcggtttaaccgcggcggaaaaacgcaaaggggtggtggcctgttcggcgggcaaccatgcgcagggggtctcgctctcctgcgccatgctcggcattgacgggaaagtggtgatgccgaaaggggcgccgaaatcgaaagtcgccgccacctgcgattattcggcagaggtagtcctgcatggcgataactttaacgataccctcgccaaagccagcgatattgttgaacttgagggccgtatttttattcccccctatgacgacccgcaggttattgccgggcagggaacgattggtctcgaaatattagaagatctgtatgacgtggataatgtcatcgtgccgattggcggcgggggattaattgccggcatcgcgattgcgattaaatccattaacccgacgatccgcattattggcgtgcagtcagaaaatgttcacgggatggccgcctcctggtatgccggggagatcaccagccatcgccacgccggcaccttagccgatggttgcgatgtcgcccggccagggaaactgacttatgaaatcgcccgccagctggtggatgacatcgtcctggtcagtgaggacgacattcgccagagcatggtcgccttaattcagcgcaataaagtgatcaccgaaggggccggggcgttggcctgcgccgcgttattaagcggcaaactagacagctatatccagaaccgcaaaacggtcagcctgatttccgggggcaatatcgatctctcgcgggtatcgcaaattacgggttttgttgacgcttaa

SEQ ID NO: 15 - protein, 330 aa, Escherichia coli
MAHITYDLPVAIDDIIEAKQRLAGRIYKTGMPRSNYFSERCKGEIFLKFENMQRTGSFKIRGAFNKLSSLTDAEKRKGVVACSAGNHAQGVSLSCAMLGIDGKVVMPKGAPKSKVAATCDYSAEVVLHGDNFNDTIAKVSEIVEMEGRIFIPPYDDPKVIAGQGTIGLEIMEDLYDVDNVIVPIGGGGLIAGIAVAIKSINPTIRVIGVQSENVHGMAASFHSGEITTHRTTGTLADGCDVSRPGNLTYEIVRELVDDIVLVSEDEIRNSMIALIQRNKVVTEGAGALACAALLSGKLDQYIQNRKTVSIISGGNIDLSRVSQITGFVDA

SEQ ID NO: 16 - DNA, 993 bp, Escherichia coli
atggctcatattacatacgatctgccggttgctattgatgacattattgaagcgaaacaacgactggctgggcgaatttataaaacaggcatgcctcgctccaactattttagtgaacgttgcaaaggtgaaatattcctgaagtttgaaaatatgcagcgtacgggttcatttaaaattcgtggcgcatttaataaattaagttcactgaccgatgcggaaaaacgcaaaggcgtggtggcctgttctgcgggcaaccatgcgcaaggggtttccctctcctgcgcgatgctgggtatcgacggtaaagtggtgatgccaaaaggtgcgccaaaatccaaagtagcggcaacgtgcgactactccgcagaagtcgttctgcatggtgataacttcaacgacactatcgctaaagtgagcgaaattgtcgaaatggaaggccgtatttttatcccaccttacgatgatccgaaagtgattgctggccagggaacgattggtctggaaattatggaagatctctatgatgtcgataacgtgattgtgccaattggtggtggcggtttaattgctggtattgcggtggcaattaaatctattaacccgaccattcgtgttattggcgtacagtctgaaaacgttcacggcatggcggcttctttccactccggagaaataaccacgcaccgaactaccggcaccctggcggatggttgtgatgtctcccgcccgggtaatttaacttacgaaatcgttcgtgaattagtcgatgacatcgtgctggtcagcgaagacgaaatcagaaacagtatgattgccttaattcagcgcaataaagtcgtcaccgaaggcgcaggcgctctggcatgtgctgcattattaagcggtaaattagaccaatatattcaaaacagaaaaaccgtcagtattatttccggcggcaatatcgatctttctcgcgtctctcaaatcaccggtttcgttgacgcctaa

SEQ ID NO: 17 - protein, 514 aa, Escherichia coli [sequence omitted]

SEQ ID NO: 18 - DNA, 1545 bp, Escherichia coli
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 19 - protein, 887 aa, Escherichia coli
MSERFPNDVDPIETRDWLQAIESVIREEGVERAQYLIDQLLAEARKGGVNVAAGTGISNYINTIPVEEQPEYPGNLELERRIRSAIRWNAIMTVLRASKKDLELGGHMASFQSSATIYDVCFNHFFRARNEQDGGDLVYFQGHISPGVYARAFLEGRLTQEQLDNFRQEVHGNGLSSYPHPKLMPEFWQFPTVSMGLGPIGAIYQAKFLKYLEHRGLKDTSKQTVYAFLGDGEMDEPESKGAITIATREKLDNLVFVINCNLQRLDGPVTGNGKIINELEGIFEGAGWNVIKVMWGSRWDELLRKDTSGKLIQLMNETVDGDYQTFKSKDGAYVREHFFGKYPETAALVADWTDEQIWALNRGGHDPKKIYAAFKKAQETKGKATVILAHTIKGYGMGDAAEGKNIAHQVKKMNMDGVRHIRDRFNVPVSDADIEKLPYITFPEGSEEHTYLHAQRQKLHGYLPSRQPNFTEKLELPSLQDFGALLEEQSKEISTTIAFVRALNVMLKNKSIKDRLVPIIADEARTFGMEGLFRQIGIYSPNGQQYTPQDREQVAYYKEDEKGQILQEGINELGAGCSWLAAATSYSTNNLPMIPFYIYYSMFGFQRIGDLCWAAGDQQARGFLIGGTSGRTTLNGEGLQHEDGHSHIQSLTIPNCISYDPAYAYEVAVIMHDGLERMYGEKQENVYYYITTLNENYHMPAMPEGAEEGIRKGIYKLETIEGSKGKVQLLGSGSILRHVREAAEILAKDYGVGSDVYSVTSFTELARDGQDCERWNMLHPLETPRVPYIAQVMNDAPAVASTDYMKLFAEQVRTYVPADDYRVLGTDGFGRSDSRENLRHHFEVDASYVVVAALGELAKRGEIDKKVVADAIAKFNIDADKVNPRLA

SEQ ID NO: 20 - DNA, 2664 bp, Escherichia coli
atgtcagaacgtttcccaaatgacgtggatccgatcgaaactcgcgactggctccaggcgatcgaatcggtcatccgtgaagaaggtgttgagcgtgctcagtatctgatcgaccaactgcttgctgaagcccgcaaaggcggtgtaaacgtagccgcaggcacaggtatcagcaactacatcaacaccatccccgttgaagaacaaccggagtatccgggtaatctggaactggaacgccgtattcgttcagctatccgctggaacgccatcatgacggtgctgcgtgcgtcgaaaaaagacctcgaactgggcggccatatggcgtccttccagtcttccgcaaccatttatgatgtgtgctttaaccacttcttccgtgcacgcaacgagcaggatggcggcgacctggtttacttccagggccacatctccccgggcgtgtacgctcgtgctttcctggaaggtcgtctgactcaggagcagctggataacttccgtcaggaagttcacggcaatggcctctcttcctatccgcacccgaaactgatgccggaattctggcagttcccgaccgtatctatgggtctgggtccgattggtgctatttaccaggctaaattcctgaaatatctggaacaccgtggcctgaaagatacctctaaacaaaccgtttacgcgttcctcggtgacggtgaaatggacgaaccggaatccaaaggtgcgatcaccatcgctacccgtgaaaaactggataacctggtcttcgttatcaactgtaacctgcagcgtcttgacggcccggtcaccggtaacggcaagatcatcaacgaactggaaggcatcttcgaaggtgctggctggaacgtgatcaaagtgatgtggggtagccgttgggatgaactgctgcgtaaggataccagcggtaaactgatccagctgatgaacgaaaccgttgacggcgactaccagaccttcaaatcgaaagatggtgcgtacgttcgtgaacacttcttcggtaaatatcctgaaaccgcagcactggttgcagactggactgacgagcagatctgggcactgaaccgtggtggtcacgatccgaagaaaatctacgctgcattcaagaaagcgcaggaaaccaaaggcaaagcgacagtaatccttgctcataccattaaaggttacggcatgggcgacgcggctgaaggtaaaaacatcgcgcaccaggttaagaaaatgaacatggacggtgtgcgtcatatccgcgaccgtttcaatgtgccggtgtctgatgcagatatcgaaaaactgccgtacatcaccttcccggaaggttctgaagagcatacctatctgcacgctcagcgtcagaaactgcacggttatctgccaagccgtcagccgaacttcaccgagaagcttgagctgccgagcctgcaagacttcggcgcgctgttggaagagcagagcaaagagatctctaccactatcgctttcgttcgtgctctgaacgtgatgctgaagaacaagtcgatcaaagatcgtctggtaccgatcatcgccgacgaagcgcgtactttcggtatggaaggtctgttccgtcagattggtatttacagcccgaacggtcagcagtacaccccgcaggaccgcgagcaggttgcttactataaagaagacgagaaaggtcagattctgcaggaagggatcaacgagctgggcgcaggttgttcctggctggcagcggcgacctcttacagcaccaacaatctgccgatgatcccgttctacatctattactcgatgttcggcttccagcgtattggcgatctgtgctgggcggctggcgaccagcaagcgcgtggcttcctgatcggcggtacttccggtcgtaccaccctgaacggcgaaggtctgcagcacgaagatggtcacagccacattcagtcgctgactatcccgaactgtatctcttacgacccggcttacgcttacgaagttgctgtcatcatgcatgacggtctggagcgtatgtacggtgaaaaacaagagaacgtttactactacatcactacgctgaacgaaaactaccacatgccggcaatgccggaaggtgctgaggaaggtatccgtaaaggtatctacaaactcgaaactattgaaggtagcaaaggtaaagttcagctgctcggctccggttctatcctgcgtcacgtccgtgaagcagctgagatcctggcgaaagattacggcgtaggttctgacgtttatagcgtgacctccttcaccgagctggcgcgtgatggtcaggattgtgaacgctggaacatgctgcacccgctggaaactccgcgcgttccgtatatcgctcaggtgatgaacgacgctccggcagtggcatctaccgactatatgaaactgttcgctgagcaggtccgtacttacgtaccggctgacgactaccgcgtactgggtactgatggcttcggtcgttccgacagccgtgagaacctgcgtcaccacttcgaagttgatgcttcttatgtcgtggttgcggcgctgggcgaactggctaaacgtggcgaaatcgataagaaagtggttgctgacgcaatcgccaaattcaacatcgatgcagataaagttaacccgcgtctggcgtaa

SEQ ID NO: 21 - protein, 630 aa, Escherichia coli
MAIEIKVPDIGADEVEITEILVKVGDKVEAEQSLITVEGDKASMEVPSPQAGIVKEIKVSVGDKTQTGALIMIFDSADGAADAAPAQAEEKKEAAPAAAPAAAAAKDVNVPDIGSDEVEVTEILVKVGDKVEAEQSLITVEGDKASMEVPAPFAGTVKEIKVNVGDKVSTGSLIMVFEVAGEAGAAAPAAKQEAAPAAAPAPAAGVKEVNVPDIGGDEVEVTEVMVKVGDKVAAEQSLITVEGDKASMEVPAPFAGVVKELKVNVGDKVKTGSLIMIFEVEGAAPAAAPAKQEAAAPAPAAKAEAPAAAPAAKAEGKSEFAENDAYVHATPLIRRLAREFGVNLAKVKGTGRKGRILREDVQAYVKEAIKRAEAAPAATGGGIPGMLPWPKVDFSKFGEIEEVELGRIQKISGANLSRNWVMIPHVTHFDKTDITELEAFRKQQNEEAAKRKLDVKITPVVFIMKAVAAALEQMPRFNSSLSEDGQRLTLKKYINIGVAVDTPNGLVVPVFKDVNKKGIIELSRELMTISKKARDGKLTAGEMQGGCFTISSIGGLGTTHFAPIVNAPEVAILGVSKSAMEPVWNGKEFVPRLMLPISLSFDHRVIDGADGARFITIINNTLSDIRRLVM

SEQ ID NO: 22 - DNA, 1893 bp, Eshcerichia coli
atggctatcgaaatcaaagtaccggacatcggggctgatgaagttgaaatcaccgagatcctggtcaaagtgggcgacaaagttgaagccgaacagtcgctgatcaccgtagaaggcgacaaagcctctatggaagttccgtctccgcaggcgggtatcgttaaagagatcaaagtctctgttggcgataaaacccagaccggcgcactgattatgattttcgattccgccgacggtgcagcagacgctgcacctgctcaggcagaagagaagaaagaagcagctccggcagcagcaccagcggctgcggcggcaaaagacgttaacgttccggatatcggcagcgacgaagttgaagtgaccgaaatcctggtgaaagttggcgataaagttgaagctgaacagtcgctgatcaccgtagaaggcgacaaggcttctatggaagttccggctccgtttgctggcaccgtgaaagagatcaaagtgaacgtgggtgacaaagtgtctaccggctcgctgattatggtcttcgaagtcgcgggtgaagcaggcgcggcagctccggccgctaaacaggaagcagctccggcagcggcccctgcaccagcggctggcgtgaaagaagttaacgttccggatatcggcggtgacgaagttgaagtgactgaagtgatggtgaaagtgggcgacaaagttgccgctgaacagtcactgatcaccgtagaaggcgacaaagcttctatggaagttccggcgccgtttgcaggcgtcgtgaaggaactgaaagtcaacgttggcgataaagtgaaaactggctcgctgattatgatcttcgaagttgaaggcgcagcgcctgcggcagctcctgcgaaacaggaagcggcagcgccggcaccggcagcaaaagctgaagccccggcagcagcaccagctgcgaaagcggaaggcaaatctgaatttgctgaaaacgacgcttatgttcacgcgactccgctgatccgccgtctggcacgcgagtttggtgttaaccttgcgaaagtgaagggcactggccgtaaaggtcgtatcctgcgcgaagacgttcaggcttacgtgaaagaagctatcaaacgtgcagaagcagctccggcagcgactggcggtggtatccctggcatgctgccgtggccgaaggtggacttcagcaagtttggtgaaatcgaagaagtggaactgggccgcatccagaaaatctctggtgcgaacctgagccgtaactgggtaatgatcccgcatgttactcacttcgacaaaaccgatatcaccgagttggaagcgttccgtaaacagcagaacgaagaagcggcgaaacgtaagctggatgtgaagatcaccccggttgtcttcatcatgaaagccgttgctgcagctcttgagcagatgcctcgcttcaatagttcgctgtcggaagacggtcagcgtctgaccctgaagaaatacatcaacatcggtgtggcggtggataccccgaacggtctggttgttccggtattcaaagacgtcaacaagaaaggcatcatcgagctgtctcgcgagctgatgactatttctaagaaagcgcgtgacggtaagctgactgcgggcgaaatgcagggcggttgcttcaccatctccagcatcggcggcctgggtactacccacttcgcgccgattgtgaacgcgccggaagtggctatcctcggcgtttccaagtccgcgatggagccggtgtggaatggtaaagagttcgtgccgcgtctgatgctgccgatttctctctccttcgaccaccgcgtgatcgacggtgctgatggtgcccgtttcattaccatcattaacaacacgctgtctgacattcgccgtctggtgatgtaa

SEQ ID NO: 23 - protein, 474 aa, Escherichia coli [sequence omitted]

SEQ ID NO: 24 - DNA, 1425 bp, Eshcerichia coli
atgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaa

SEQ ID NO: 25 - protein, 330 aa, Bacillus subtilis
MSTNRHQALGLTDQEAVDMYRTMLLARKIDERMWLLNRSGKIPFVISCQGQEAAQVGAAFALDREMDYVLPYYRDMGVVLAFGMTAKDLMMSGFAKAADPNSGGRQMPGHFGQKKNRIVTGSSPVTTQVPHAVGIALAGRMEKKDIAAFVTFGEGSSNQGDFHEGANFAAVHKLPVIFMCENNKYAISVPYDKQVACENISDRAIGYGMPGVTVNGNDPLEVYQAVKEARERARRGEGPTLIETISYRLTPHSSDDDDSSYRGREEVEEAKKSDPLLTYQAYLKETGLLSDEIEQTMLDEIMAIVNEATDEAENAPYAAPESALDYVYAK

SEQ ID NO: 26 - DNA, 993 bp, Bacillus subtilis
atgagtacaaaccgacatcaagcactagggctgactgatcaggaagccgttgatatgtatagaaccatgctgttagcaagaaaaatcgatgaaagaatgtggctgttaaaccgttctggcaaaattccatttgtaatctcttgtcaaggacaggaagcagcacaggtaggagcggctttcgcacttgaccgtgaaatggattatgtattgccgtactacagagacatgggtgtcgtgctcgcgtttggcatgacagcaaaggacttaatgatgtccgggtttgcaaaagcagcagatccgaactcaggaggccgccagatgccgggacatttcggacaaaagaaaaaccgcattgtgacgggatcatctccggttacaacgcaagtgccgcacgcagtcggtattgcgcttgcgggacgtatggagaaaaaggatatcgcagcctttgttacattcggggaagggtcttcaaaccaaggcgatttccatgaaggggcaaactttgccgctgtccataagctgccggttattttcatgtgtgaaaacaacaaatacgcaatctcagtgccttacgataagcaagtcgcatgtgagaacatttccgaccgtgccataggctatgggatgcctggcgtaactgtgaatggaaatgatccgctggaagtttatcaagcggttaaagaagcacgcgaaagggcacgcagaggagaaggcccgacattaattgaaacgatttcttaccgccttacaccacattccagtgatgacgatgacagcagctacagaggccgtgaagaagtagaggaagcgaaaaaaagtgatcccctgcttacttatcaagcttacttaaaggaaacaggcctgctgtccgatgagatagaacaaaccatgctggatgaaattatggcaatcgtaaatgaagcgacggatgaagcggagaacgccccatatgcagctcctgagtcagcgcttgattatgtttatgcgaagtag

SEQ ID NO: 27 - protein, 392 aa, Bacillus subtilis
MSVMSYIDAINLAMKEEMERDSRVFVLGEDVGRKGGVFKATAGLYEQFGEERVMDTPLAESAIAGVGIGAAMYGMRPIAEMQFADFIMPAVNQIISEAAKIRYRSNNDWLLNRSGKIPFVISCPIVVRAPYGGGVHGALYHSQSVEAIFANQPGLKIVMPSTPYDAKGLLKAAVRDEDPVLAFFEHKDLMMSGFAKAADPNSGGRAYRLIKGEVPADDYVLPIGKYAISVPYDKQVACENISDVKREGDDIGYGMPGVTVITYGLCVHFALQAAERLEKDGISAHVVDPLRTVYPLDKEAIIEAASKTGKVLTYQAYLVTEDTKETGSIMSEVAAIISEHCLFDLSDAPIKRLDEIMAGPDIPAMPYAPTMEKYFMVNPDKVEAAMRELAEF

SEQ ID NO: 28 - DNA, 984 bp, Bacillus subtilis
atgtcagtaatgtcatatattgatgcaatcaatttggcgatgaaagaagaaatggaacgagattctcgcgttttcgtccttggggaagatgtaggaagaaaaggcggtgtgtttaaagcgacagcgggactctatgaacaatttggggaagagcgcgttatggatacgccgcttgctgaatctgcaatcgcaggagtcggtatcggagcggcaatgtacggaatgagaccgattgctgaaatgcagtttgctgatttcattatgccggcagtcaaccaaattatttctgaagcggctaaaatccgctaccgcagcaacaatgactggagctgtccgattgtcgtcagagcgccatacggcggaggcgtgcacggagccctgtatcattctcaatcagtcgaagcaattttcgccaaccagcccggactgaaaattgtcatgccatcaacaccatatgacgcgaaagggctcttaaaagccgcagttcgtgacgaagaccccgtgctgttttttgagcacaagcgggcataccgtctgataaagggcgaggttccggctgatgattatgtcctgccaatcggcaaggcggacgtaaaaagggaaggcgacgacatcacagtgatcacatacggcctgtgtgtccacttcgccttacaagctgcagaacgtctcgaaaaagatggcatttcagcgcatgtggtggatttaagaacagtttacccgcttgataaagaagccatcatcgaagctgcgtccaaaactggaaaggttcttttggtcacagaagatacaaaagaaggcagcatcatgagcgaagtagccgcaattatatccgagcattgtctgttcgacttagacgcgccgatcaaacggcttgcaggtcctgatattccggctatgccttatgcgccgacaatggaaaaatactttatggtcaaccctgataaagtggaagcggcgatgagagaattagcggagttttaa

SEQ ID NO: 29 - protein, 424 aa, Bacillus subtilis
MAIEQMTMPQLGESVTEGTISKWLVAPGDKVNKYDPIAEVMTDKVNAEVPSSFTGTITELVGEEGQTLQVGEMICKIETEGANPAEQKQEQPAASEAAENPVAKSAGAADQPNKKRYSPAVLRLAGEHGIDLDQVTGTGAGGRITRKDIQRLIETGGVQEQNPEELKTAAPAPKSASKPEPKEETSYPASAAGDKEIPVTGVRKAIASNMKRSKTEIPHAWTMMEVDVTNMVAYRNSIKDSFKKTEGFNLTFFAFFVKAVAQALKEFPQMNSMWAGDKIIQKKDINISIAVATEDSLFVPVIKNADEKTIKGIAKDITGLAKKVRDGKLTADDMQGGTFTVNNTGSFGSVQSMGIINYPQAAILQVESIVKRPVVMDNGMIAVRDMVNLCLSLDHRVLDGLVCGRFLGRVKQILESIDEKTSVY

SEQ ID NO: 30 - DNA, 1275 bp, Bacillus subtilis
atggcaattgaacaaatgacgatgccgcagcttggagaaagcgtaacagaggggacgatcagcaaatggcttgtcgcccccggtgataaagtgaacaaatacgatccgatcgcggaagtcatgacagataaggtaaatgcagaggttccgtcttcttttactggtacgataacagagcttgtgggagaagaaggccaaaccctgcaagtcggagaaatgatttgcaaaattgaaacagaaggcgcgaatccggctgaacaaaaacaagaacagccagcagcatcagaagccgctgagaaccctgttgcaaaaagtgctggagcagccgatcagcccaataaaaagcgctactcgccagctgttctccgtttggccggagagcacggcattgacctcgatcaagtgacaggaactggtgccggcgggcgcatcacacgaaaagatattcagcgcttaattgaaacaggcggcgtgcaagaacagaatcctgaggagctgaaaacagcagctcctgcaccgaagtctgcatcaaaacctgagccaaaagaagagacgtcatatcctgcgtctgcagccggtgataaagaaatccctgtcacaggtgtaagaaaagcaattgcttccaatatgaagcgaagcaaaacagaaattccgcatgcttggacgatgatggaagtcgacgtcacaaatatggttgcatatcgcaacagtataaaagattcttttaagaagacagaaggctttaatttaacgttcttcgccttttttgtaaaagcggtcgctcaggcgttaaaagaattcccgcaaatgaatagcatgtgggcgggggacaaaattattcagaaaaaggatatcaatatttcaattgcagttgccacagaggattctttatttgttccggtgattaaaaacgctgatgaaaaaacaattaaaggcattgcgaaagacattaccggcctagctaaaaaagtaagagacggaaaactcactgcagatgacatgcagggaggcacgtttaccgtcaacaacacaggttcgttcgggtctgttcagtcgatgggcattatcaactaccctcaggctgcgattcttcaagtagaatccatcgtcaaacgcccggttgtcatggacaatggcatgattgctgtcagagacatggttaatctgtgcctgtcattagatcacagagtgcttgacggtctcgtgtgcggacgattcctcggacgagtgaaacaaattttagaatcgattgacgagaagacatctgtttactaa

SEQ ID NO: 31 - protein, 474 aa, Bacillus subtilis
MATEYDVVILGGGTGGYVAAIRAAQLGLKTAVVEKEKLGGTCLHKGCIPSKALLRSAEVYRTAREADQFGVETAGVSLNFEKVQQRKQAVVDKLAAGVNHLMKKGKIDVYTGYGRILGPSIFSPLPGTISVERGNGEENDMLIPKQVIIATGSRPRMLPGLEVDGKSVLTSDEALQMEELPQSIIIVGGGVIGIEWASMLHDFGVKVTVIEYADRILPTEDLEISKEMESLLKKKGIQFITGAKVLPDTMTKTSDDISIQAEKDGETVTYSAEKMLVSIGRQANIEGIGLENTDIVTENGMISVNESCQTKESHIYAIGDVIGGLQLAHVASHEGIIAVEHFAGLNPHPLDPTLVPKCIYSSPEAASVGLTEDEAKANGHNVKIGKFPFMAIGKALVYGESDGFVKIVADRDTDDILGVHMIGPHVTDMISEAGLAKVLDATPWEVGQTIHPHPTLSEAIGEAALAADGKAIHF

SEQ ID NO: 32 - DNA, 1425 bp, Bacillus subtilis
atggcaactgagtatgacgtagtcattctgggcggcggtaccggcggttatgttgcggccatcagagccgctcagctcggcttaaaaacagccgttgtggaaaaggaaaaactcgggggaacatgtctgcataaaggctgtatcccgagtaaagcgctgcttagaagcgcagaggtataccggacagctcgtgaagccgatcaattcggagtggaaacggctggcgtgtccctcaactttgaaaaagtgcagcagcgtaagcaagccgttgttgataagcttgcagcgggtgtaaatcatttaatgaaaaaaggaaaaattgacgtgtacaccggatatggacgtatccttggaccgtcaatcttctctccgctgccgggaacaatttctgttgagcggggaaatggcgaagaaaatgacatgctgatcccgaaacaagtgatcattgcaacaggatcaagaccgagaatgcttccgggtcttgaagtggacggtaagtctgtactgacttcagatgaggcgctccaaatggaggagctgccacagtcaatcatcattgtcggcggaggggttatcggtatcgaatgggcgtctatgcttcatgattttggcgttaaggtaacggttattgaatacgcggatcgcatattgccgactgaagatctagagatttcaaaagaaatggaaagtcttcttaagaaaaaaggcatccagttcataacaggggcaaaagtgctgcctgacacaatgacaaaaacatcagacgatatcagcatacaagcggaaaaagacggagaaaccgttacctattctgctgagaaaatgcttgtttccatcggcagacaggcaaatatcgaaggcatcggcctagagaacaccgatattgttactgaaaatggcatgatttcagtcaatgaaagctgccaaacgaaggaatctcatatttatgcaatcggagacgtaatcggtggcctgcagttagctcacgttgcttcacatgagggaattattgctgttgagcattttgcaggtctcaatccgcatccgcttgatccgacgcttgtgccgaagtgcatttactcaagccctgaagctgccagtgtcggcttaaccgaagacgaagcaaaggcgaacgggcataatgtcaaaatcggcaagttcccatttatggcgattggaaaagcgcttgtatacggtgaaagcgacggttttgtcaaaatcgtggctgaccgagatacagatgatattctcggcgttcatatgattggcccgcatgtcaccgacatgatttctgaagcgggtcttgccaaagtgctggacgcaacaccgtgggaggtcgggcaaacgattcacccgcatccaacgctttctgaagcaattggagaagctgcgcttgccgcagatggcaaagccattcatttttaa

SEQ ID NO: 33 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 34 - DNA, 1699 bp, Lactococcus lactis
gaattcgcggccgcttctagaaggagatatacatatgtataccgtgggtgactacctgctggaccgtctgcatgaactgggcattgaagaaatctttggtgttccgggtgactacaacctgcaatttctggatcaaattatctcacgtgaagacatgaaatggattggtaacgcaaatgaactgaacgcatcgtatatggctgatggctacgcgcgcaccaaaaaagcggcggcgtttctgaccacgttcggcgttggtgaactgagcgcgattaacggcctggccggttcttatgcagaaaatctgccggtggttgaaatcgttggctcaccgacgtcgaaagtccagaatgatggtaaatttgtgcatcacaccctggcggatggcgactttaaacatttcatgaaaatgcacgaaccggtgacggctgcgcgtaccctgctgacggcggaaaacgccacctatgaaattgatcgtgtgctgagtcaactgctgaaagaacgcaaaccggtttacatcaatctgccggttgacgtcgccgcagctaaagctgaaaaaccggcgctgtccctggaaaaagaaagctctaccacgaacaccacggaacaggttattctgagcaaaatcgaagaatctctgaaaaatgcccaaaaaccggtcgtgattgcaggccatgaagtgatcagttttggtctggaaaaaaccgtcacgcagttcgtgtccgaaaccaaactgccgattaccacgctgaactttggtaaaagcgccgtggatgaaagcctgccgtctttcctgggcatttataacggtaaactgagtgaaatctccctgaaaaacttcgtcgaatctgctgatttcatcctgatgctgggcgtgaaactgaccgacagttccacgggtgcctttacccatcacctggatgaaaacaaaatgattagcctgaatatcgacgaaggcatcatcttcaacaaagttgtcgaagatttcgacttccgtgcggtggtttcatcgctgtctgaactgaaaggcattgaatatgaaggccagtacatcgataaacaatacgaagaatttatcccgagcagcgcaccgctgagtcaggaccgtctgtggcaagcagttgaatcactgacgcagtcgaacgaaaccattgtcgctgaacaaggcaccagctttttcggtgcgtccaccatctttctgaaaagtaattcccgtttcattggtcagccgctgtggggcagcatcggttatacctttccggcggccctgggctcacaaattgccgataaagaatcgcgccatctgctgttcatcggcgacggcagcctgcaactgaccgttcaagaactgggtctgtcgattcgtgaaaaactgaacccgatctgctttattatcaacaatgatggctacacggtggaacgcgaaattcacggtccgacccagagttataacgacatcccgatgtggaattactccaaactgccggaaacgtttggcgcaaccgaagatcgtgtcgtgagcaaaattgtgcgcaccgaaaacgaatttgtgtctgttatgaaagaagcacaggctgatgttaatcgcatgtattggatcgaactggtcctggaaaaagaagatgctccgaaactgctgaaaaaaatgggtaaactgttcgctgaacaaaataaataatactagtagcggccgctgcag

SEQ ID NO: 35 - protein, 464 aa, Salmonella enterica
MNTSELETLIRTILSEQLTTPAQTPVQPQGKGIFQSVSEAIDAAHQAFLRYQQCPLKTRSAIISAMRQELTPLLAPLAEESANETGMGNKEDKFLKNKAALDNTPGVEDLTTTALTGDGGMVLFEYSPFGVIGSVAPSTNPTETIINNSISMLAAGNSIYFSPHPGAKKVSLKLISLIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAVCLPEGQANKKLVGKSPSAMLEAAGIAVPAKAPRLLIALVNADDPWVTSEQLMPMLPVVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 36 - DNA, 1450 bp, Salmonella enterica
gaattcgcggccgcttctagaaggagatatacatatgaacacctcggaactggaaaccctgattcgcaccatcctgtcggaacaactgaccaccccggctcaaaccccggtccaaccgcagggcaaaggtatctttcagagcgtttctgaagcaattgatgcggcccatcaggcgtttctgcgttatcagcaatgcccgctgaaaacgcgtagcgctattatctctgcgatgcgtcaggaactgaccccgctgctggctccgctggcggaagaaagtgcgaacgaaaccggcatgggtaacaaagaagataaattcctgaagaacaaggcagctctggataatacgccgggtgtcgaagacctgaccacgaccgcactgaccggtgatggtggtatggtgctgtttgaatatagcccgttcggtgtgattggcagtgttgcaccgtccaccaacccgacggaaaccattatcaacaatagtatctccatgctggcggcgggcaacagcatttacttttcgccgcatccgggcgcgaaaaaggtttcactgaaactgatttcgctgatcgaagaaattgcctttcgttgctgtggtatccgcaacctggtggttacggtggccgaaccgacgtttgaagcaacccagcaaatgatggctcacccgcgtatcgcagtcctggcaattaccggcggtccgggcattgtggcgatgggtatgaaaagcggcaaaaaggttatcggtgcaggtgcaggtaatccgccgtgcattgttgatgaaaccgccgacctggtcaaagcggcggaagatattatcaacggtgcctcttttgactataatctgccgtgtatcgcagaaaagagcctgattgtcgtggaatctgtcgcggaacgtctggtgcagcaaatgcagacgttcggcgcgctgctgctgtccccggcggataccgacaaactgcgtgcagtttgcctgccggagggtcaggccaacaaaaagctggtcggcaaatcaccgtcggcaatgctggaagcggcgggtatcgctgtgccggcaaaggctccgcgtctgctgattgccctggtgaatgcagatgacccgtgggttacctctgaacaactgatgccgatgctgccggttgtcaaagtgagcgattttgactctgcgctggccctggcactgaaggttgaagaaggcctgcatcacaccgcgattatgcacagtcagaacgtttcccgtctgaatctggcagctcgcacgctgcaaacctcaatcttcgtcaaaaacggtccgtcgtacgcaggtattggcgtgggcggtgaaggctttacgaccttcaccatcgcaacgccgaccggtgaaggcacgaccagtgctcgtacgtttgcgcgctcccgtcgctgtgtgctgaccaatggtttcagcattcgctaatactagtagcggccgctgcag

SEQ ID NO: 37 - protein, 764 aa, Escherichia coli
MKVDIDTSDKLYADAWLGFKGTDWKNEINVRDFIQHNYTPYEGDESFLAEATPATTELWEKVMEGIRIENATHAPVDFDTNIATTITAHDAGYINQPLEKIVGLQTDAPLKRALHPFGGINMIKSSFHAYGREMDSEFEYLFTDLRKTHNQGVFDVYSPDMLRCRKSGVLTGLPDGYGRGRIIGDYRRVALYGISYLVRERELQFADLQSRLEKGEDLEATIRLREELAEHRHALLQIQEMAAKYGFDISRPAQNAQEAVQWLYFAYLAAVKSQNGGAMSLGRTASFLDIYIERDFKAGVLNEQQAQELIDHFIMKIRMVRFLRTPEFDSLFSGDPIWATEVIGGMGLDGRTLVTKNSFRYLHTLHTMGPAPEPNLTILWSEELPIAFKKYAAQVSIVTSSLQYENDDLMRTDFNSDDYAIACCVSPMVIGKQMQFFGARANLAKTLLYAINGGVDEKLKIQVGPKTAPLMDDVLDYDKVMDSLDHFMDWLAVQYISALNIIHYMHDKYSYEASLMALHDRDVYRTMACGIAGLSVATDSLSAIKYARVKPIRDENGLAVDFEIDGEYPQYGNNDERVDSIACDLVERFMKKIKALPTYRNAVPTQSILTITSNVVYGQKTGNTPDGRRAGTPFAPGANPMHGRDRKGAVASLTSVAKLPFTYAKDGISYTFSIVPAALGKEDPVRKTNLVGLLDGYFHHEADVEGGQHLNVNVMNREMLLDAIEHPEKYPNLTIRVSGYAVRFNALTREQQQDVISRTFTQAL

SEQ ID NO: 38 - DNA, 2295 bp, Escherichia coli
atgaaggtagatattgataccagcgataagctgtacgccgacgcatggcttggctttaaaggtacggactggaaaaacgaaattaatgtccgcgattttattcaacataactatacaccgtatgaaggcgatgaatctttcctcgccgaagcgacgcctgccaccacggaattgtgggaaaaagtaatggaaggcatccgtatcgaaaatgcaacccacgcgccggttgatttcgataccaatattgccaccacaattaccgctcatgatgcgggatatattaaccagccgctggaaaaaattgttggcctgcaaacggatgcgccgttgaaacgtgcgctacacccgttcggtggcattaatatgattaaaagttcattccacgcctatggccgagaaatggacagtgaatttgaatatctgtttaccgatctgcgtaaaacccataaccagggcgtatttgatgtttactcaccggatatgctgcgctgccgtaaatctggcgtgctgaccggtttaccagatggctatggccgtgggcgcattatcggtgactatcgccgcgtagcgctgtatggcatcagttatctggtacgtgaacgcgaactgcaatttgccgatctccagtctcgtctggaaaaaggcgaggatctggaagccaccatccgtctgcgtgaggagctggcagagcatcgtcatgcgctgttgcagattcaggaaatggcggcgaaatatggctttgatatctctcgcccggcgcagaatgcgcaggaagcggtgcagtggctctacttcgcttatctggcggcagtgaaatcgcaaaatggcggcgcgatgtcgctgggccgcacggcatcgttcctcgatatctacattgagcgcgactttaaagctggcgtactcaatgagcagcaggcacaggaactgatcgatcacttcatcatgaagatccgtatggtacgcttcctgcgtacaccggaatttgattcgctgttctccggcgacccaatctgggcgacggaagtgatcggcgggatggggctggacggtcgtacgctggtgaccaaaaactccttccgctatttgcacaccctgcacactatggggccggcaccggaacctaacctgaccattctttggtcggaagaattaccgattgccttcaaaaaatatgccgcgcaggtgtcgatcgtcacctcttccttgcagtatgaaaatgacgatctgatgcgtactgacttcaacagcgacgattacgcgattgcctgctgcgtcagcccaatggtgattggtaagcaaatgcagttctttggtgcacgcgctaacctggcgaaaacgctgctctacgcaattaacggcggggtggacgagaagctgaagattcaggtcgggccgaaaacagcaccgctgatggacgacgtgctggattacgacaaagtgatggacagcctcgatcacttcatggactggctggcggtgcagtacatcagcgcgctgaatatcattcactacatgcacgacaagtacagctacgaagcttcgctgatggcgctgcacgatcgtgatgtctatcgcactatggcatgcggcatcgcgggcctgtcggtggcgacggactccctgtctgccatcaaatatgcccgcgtgaaaccaatccgtgacgaaaacggcctggcggtggactttgaaatcgacggtgaatatccgcagtacggcaacaacgacgagcgcgtagacagcattgcctgcgacctggttgaacgctttatgaagaaaattaaagcgctgccaacctatcgcaacgccgtccctacccagtcgattctgactatcacttctaacgtggtgtacggccagaaaaccggtaatacgccggacggtcgtcgcgccggaacaccgttcgcgccgggcgctaacccgatgcatggtcgtgaccgcaaaggtgccgtggcctcattgacgtcggtggcgaaactgccgttcacctacgccaaagatgggatctcgtacaccttctcaatcgttcctgcggcgctgggcaaagaagatccagtacgtaaaaccaaccttgtcggcctgctggatgggtatttccaccacgaagcggatgtcgaaggcggtcaacacctcaacgtcaacgtaatgaatcgggaaatgctgctggatgccatcgagcacccggaaaaatatcctaacctgacaatccgtgtctctggctacgccgtgcgcttcaacgcactgacccgtgaacagcaacaggatgttatttcacgtacctttacccaggcgctctga

SEQ ID NO: 39 - protein, 671 aa, Janibacter sp.
MARTYAGHSSAAASNALYRRNLAKGQTGLSVAFDLPTQTGYDPDHVLARGEVGKVGVPISHIGDMRALFDQIPLGQMNTSMTINATAMWLLAMYQVAAEDQATAADEDPASVVKALGGTTQNDIIKEYLSRGTYVFAPAPSLRLITDMVSYTVSDIPKWNPINICSYHLQEAGATPVQEIAYAMSTAIAVLDAVRDAGQVPQERFGEVVARISFFVNAGVRFVEEMCKMRAFVELWDELTRERYGVTDAKQRRFRYGVQVNSLGLTEAQPENNVQRIVLEMLAVTLSKGARARAVQLPAWNEALGLPRPWDQQWSLRMQQVLAYESDLLEYEDLFEGSAVVEAKVAELVAGAKAEIARVAELGGAVAAVESGYMKSALVASHALRRQRIEAGEDIVVGVNKFETTEPNPLTADLDTAIQSVDAGVEAAAAKAVREWRETRDADPVKRERAVAALARLKAAAQTDENLMEASIECARAEVTTGEWAQALREVFGEFRAPTGVTGTVGLTGGAAGAELSAVRERVAGLRDELGETLRVLVGKPGLDGHSNGAEQIAVRARDAGFEVIYQGIRLTPEQIVAAAVSEDVHLVGISILSGSHMELIPEVLDRLREAGAGDIPVIVGGIIPESDAAKLKAIGVAEVFTPKDFGLNDIMGRFVDVIRDSRLTTAAPTV

SEQ ID NO: 40 - DNA, 1917 bp, Janibacter sp.
atggcaagcacggaccagggtaccaacccggcagacaccgacgacctgacgccaaccactctgagtctggcgggcgattttccgaaagcaaccgaagaacagtgggagcgcgaagtggagaaagttctgaaccgtggccgtccgccggagaaacagctgacgtttgcggaatgtctgaaacgcctgacggtccacacagtagacggcattgacattgtgccaatgtatcgcccgaaagatgcgccgaagaaactgggttacccaggcgttgccccatttacacgtgggaccacggttcgtaatggcgatatggacgcatgggatgtccgtgcactgcatgaagatccggatgagaaatttacgcgcaaagcgattctggaagggctggaacgcggggttacatctctgctgctgcgtgtggacccggacgctattgctccagaacacctggatgaagtgctgtctgacgtgctgctggagatgaccaaagtagaagtctttagtcgttacgatcaaggcgccgctgccgaggcgctggtatctgtgtacgagcgcagcgataaaccggctaaggacctggctctgaatctgggtctggacccgatcgccttcgcggcactgcaggggacggaacctgatctgactgtcctgggtgattgggtgcgtcgcctggcaaaatttagcccagattctcgtgcagtgaccatcgatgcgaacatttatcataatgcgggtgcgggcgatgtagcagagctggcttgggccctggctaccggtgcggaatatgttcgtgcactggtagaacaaggttttacggcgaccgaggcgttcgatacgattaactttcgtgtgaccgcaacccatgatcagtttctgacaatcgcgcgtctgcgcgcactgcgtgaggcgtgggcgcgcattggggaggtatttggggttgatgaggataaacgtggcgcccgtcaaaatgcgatcacgagttggcgcgatgtgacacgcgaggacccgtatgtgaatatcctgcgcgggagcatcgctacattttctgcaagcgtgggtggggccgaaagtattacaactctgccttttacccaggcactgggtctgccagaagacgattttccgctgcgtatcgctcgtaataccggtatcgttctggccgaagaagtgaacatcggtcgtgttaatgatccggccggcggtagctattacgtggaaagtctgactcgtagtctggccgatgcagcgtggaaagagttccaagaagtggagaaactgggcggcatgagcaaggcggtgatgacggaacatgtaacgaaagtgctggatgcctgcaatgcagaacgcgcgaaacgcctggccaatcgcaaacagccgattaccgcagtaagcgaatttcctatgattggggcgcgctctatcgaaacgaaaccttttcctgccgcaccggcccgtaaaggtctggcatggcatcgcgacagtgaagtattcgaacaactgatggatcgcagcaccagtgtgagtgaacgtccaaaggttttcctggcgtgcctgggcacacgtcgtgacttcggtggtcgtgagggttttagcagcccagtgtggcatatcgcaggcattgacaccccacaggttgagggtggcacaaccgcagaaatcgtagaagcattcaagaaatctggggcacaagttgcggatctgtgctctagcgccaaagtgtacgctcagcagggtctggaggtggccaaagctctgaaagcagctggcgccaaagccctgtatctgagcggtgcctttaaggagttcggcgatgatgcggctgaggcggagaaactgatcgatggtcgcctgtttatgggtatggatgtggttgacactctgtctagtacgctggacattctgggtgtagcaaagtaa

SEQ ID NO: 41 - protein, 546 aa, Janibacter sp.
MTVAPKRPAAMTLAAHFPERTQEQWRDLVAGVVNKGRPEDQHLSGDDAVATMRSHLEGGLDIEPLYMKSSDPVPLGVPGAMPFTRGRALRDADVPWDVRQVHDDPDAAATRQLVLADLENGVTSVWLHVGADGLAPNDVAEALAEVRLELAPVVVSSWDDQTAAADALYAVLSGSRASSGNLGHDPLGAAARTGSAPDLAPLADAVRRLADHGEIRAITVDTRVHGDAGVTVTDEVAFALATGVAYLRHLESEGVDVAEAFRNIEFRVSATADQFLTAAALRALRRAWARIGESVGVPETSRGAFTHAVTSGRIFTRDDAWTNILRSTLATFGASLGGADAITVLPFDTVSGLPTPFSRRIARNTQILLAEESNVARVTDPAGGSWYVETLTDDVAKAAWETFQEIESAGGMVAALANGLVAQRILAAVAERDAALATRSTPITGVSTFPLAGEKPLERVVRAELPVQPNALAPHRDSAIFEALRDRSAAYATEHGHAPRVSVPTLDVPRAADRRIDAVNLLTVAGIDAVDGDTESAAALTGTDKG

SEQ ID NO: 42 - DNA, 1716 bp, Janibacter sp.
atgacggtggccccgaagcggcccgcagcgatgacgctggcggcacacttcccggagcggacgcaggagcagtggcgagacctcgtcgctggcgtggtcaacaaggggcgccccgaggaccagcacctgagcggcgacgacgctgttgccacgatgcgctcgcacctcgagggtgggctcgacatcgagccgctctacatgaagtcgtcggaccccgtgccgctcggcgtgccgggtgcgatgccgttcacccgtggccgcgcactgcgtgatgccgacgtcccgtgggacgtgcgccaggtgcacgacgacccggacgctgccgcgacgcgccagctcgtcctcgccgacctcgagaacggcgtcacctctgtctggctccacgtcggtgccgacggccttgcccccaatgatgtcgcggaggcgcttgccgaggtccgcctcgaactcgccccggtcgtcgtctcctcgtgggacgaccagaccgctgccgcggacgccctgtatgccgtcctgtccggttctcgtgcgagttccggcaacctcgggcacgaccccctcggtgccgcggcacgcacgggctcagcgcccgacctggccccactggccgatgcggtccgccgtcttgccgaccatggcgagatccgggcgatcacggttgacacccgggtccacggcgatgctggagtgaccgtgaccgatgaggtcgcgttcgcgctcgccaccggtgtggcctatctccgccacctcgagtccgagggcgtcgatgtcgcggaagccttccgcaacatcgagttccgcgtgagcgccaccgccgaccagttcctcacggcggctgcgctgcgggcgttgcgccgggcctgggcgcggatcggcgagagcgtcggtgtccccgagacgtcccgtggtgccttcacccatgccgtgacgtccggtcgcatcttcacccgcgacgacgcctggaccaacatcctgcgcagcaccctcgcgacgttcggtgccagcctcggcggggcggatgccatcaccgtgctgcccttcgacaccgtgtccgggttgccgacgccgttctcccgacgcatcgctcgcaacacccagatcctgctcgccgaggagtccaacgttgcgcgggtcaccgacccggcgggtggctcctggtacgtcgagaccctcacggacgacgtggccaaggccgcgtgggagaccttccaggagatcgagtccgccggtggcatggtcgctgccctcgcgaatggccttgtcgcacagcgtattttggcggctgtcgccgagcgcgacgccgccctggcaacacgctccacgccgataacgggcgtgagcacgttcccactggctggcgagaagccgcttgagcgagtggttcgagccgagctgcccgtgcagcccaatgcccttgcgccacaccgggactcggccatcttcgaagcgctccgggaccgctctgcggcatacgcaacagagcacggtcacgctccgcgcgtctcggtgccgaccctcgacgtgcctcgcgccgccgaccgtcgcatcgacgcggtcaacctgctcaccgtcgccggaatcgacgcggtcgacggcgacaccgagtccgccgccgccctgactggcaccgacaagggctacgagggtgtcgccaaggacatggacgtcgtcgccttcctctccgacctcctcgacacgacgggagctcccgcatga

SEQ ID NO: 43 - protein, 261 aa, Escherichia coli
MSYQYVNVVTINKVAVIEFNYGRKLNALSKVFIDDLMQALSDLNRPEIRCIILRAPSGSKVFSAGHDIHELPSGGRDPLSYDDPLRQITRMIQKFPKPIISMVEGSVWGGAFEMIMSSDLIIAASTSTFSMTPVNLGVPYNLVGIHNLTRDAGFHIVKELIFTASPITAQRALAVGILNHVVEVEELEDFTLQMAHHISEKAPLAIAVIKEELRVLGEAHTMNSDEFERIQGMRRAVYDSEDYQEGMNAFLEKRKPNFVGH

SEQ ID NO: 44 - DNA, 786 bp, Escherichia coli
atgtcttatcagtatgttaacgttgtcactatcaacaaagtggcggtcattgagtttaactatggccgaaaacttaatgccttaagtaaagtctttattgatgatcttatgcaggcgttaagcgatctcaaccggccggaaattcgctgtatcattttgcgcgcaccgagtggatccaaagtcttctccgcaggtcacgatattcacgaactgccgtctggcggtcgcgatccgctctcctatgatgatccattgcgtcaaatcacccgcatgatccaaaaattcccgaaaccgatcatttcgatggtggaaggtagtgtttggggtggcgcatttgaaatgatcatgagttccgatctgatcatcgccgccagtacctcaaccttctcaatgacgcctgtaaacctcggcgtcccgtataacctggtcggcattcacaacctgacccgcgacgcgggcttccacattgtcaaagagctgatttttaccgcttcgccaatcaccgcccagcgcgcgctggctgtcggcatcctcaaccatgttgtggaagtggaagaactggaagatttcaccttacaaatggcgcaccacatctctgagaaagcgccgttagccattgccgttatcaaagaagagctgcgtgtactgggcgaagcacacaccatgaactccgatgaatttgaacgtattcaggggatgcgccgcgcggtgtatgacagcgaagattaccaggaagggatgaacgctttcctcgaaaaacgtaaacctaatttcgttggtcattaa

SEQ ID NO: 45 - protein, 497 aa, Methanobrevibacter ruminatntium
MKIEVLDTTLRDGEQTPGISLNTIKKLRIATKLDEIGVNSIEAGSAITSEGEREAIKAITSQGLNAEIVSFSRTLIKDVDYCLECDVDAVNIVVPTSDLHLQYKLKKTQDEMLEDAVKVTEYAKDHGVKVELAAEDSTRTDIQYLRKIFKATIDAGADRICPCDTLGILTPLKSFNFYKQFTDLGVPVSAHCHNDFGLAVANTLSAIDGGASRFHATINGLGERAGNAALEEVVVSLYTLYKDESNERKYETDIKIDQIYSTSKLVSRLSNAYLAPNKPIVGENAFAHESGIHADGVIKNSATYEPIMPELVGHRRKFVIGKHVGTKGLNNRLEELGLEVNKKQLNDIFYKVKDLGDKGKTVTDTDLEAIAEHVLNIEQEKKINLDELTIVSGNKIRPTASIKLNIENEEVIEADVGIGPVDAAINAVNKGIKSFADIQLEEYHVDAVTGGTDALIEVIIKLSSGDKIISARATEPDIINASVEAYIDGVNRLLENK

SEQ ID NO: 46 - DNA, 1494 bp, Methanobrevibacter ruminatntium
atgaaaatagaagtactggatacaacacttagagacggagagcaaacccctggaatatctctaaacactattaaaaagttaagaatagccacaaaactagatgagataggagtcaattcaatagaagcaggatctgcaataacctccgaaggggaaagggaagcaataaaggcaatcacctcccaaggactgaatgctgaaatcgtaagtttttcaagaaccctaataaaggatgtagattattgcttagaatgtgatgtggatgcagtcaacattgttgttccaacttctgacttgcaccttcaatacaaactaaaaaagacccaagatgaaatgcttgaagatgcagtgaaggtaacagaatacgctaaagaccatggagtcaaagtggagcttgcagctgaagactcaacaagaacagacatccaatacctaagaaaaatatttaaggcaacaatcgatgccggagcagacagaatctgcccatgcgacactttaggaatcctaacaccacttaagtcctttaacttctataagcaatttacagacttgggagttccagtaagcgcacattgccataatgactttggccttgcagttgcaaacaccttatccgctatcgatgggggagccagcagattccatgcaaccataaacggacttggggagagggctggaaacgccgcccttgaagaggttgtagtctcactatacacattatataaagacgaaagcaatgaaagaaaatacgaaacagacattaagatagatcagatttacagcacttccaaattggtttcaagattaagcaatgcatatcttgctccaaataaaccgattgtaggtgaaaatgcgtttgcacatgaatctggaatccatgcagacggagtcattaaaaacagcgcaacatatgaacctatcatgccagagcttgtaggacacagaagaaaatttgtaattggaaagcatgtgggaacaaaaggcttaaacaaccgactggaagagcttggccttgaagtaaacaagaagcaattaaatgatattttctataaggtaaaggaccttggagacaagggaaagaccgtaacagacacagatttggaagcgatagcagagcatgtcctaaacatagagcaggaaaagaaaatcaatcttgatgagctgaccatcgtatcaggtaacaagatcagaccaacagcctcaataaagttgaacattgaaaatgaagaggtaatagaggctgatgtaggtataggtcctgtagatgctgcaataaatgctgtgaataagggaattaaaagctttgcagacattcagcttgaagagtaccatgtagatgcagttacaggaggtacagatgcactcattgaagtaatcatcaagctcagcagcggagataagatcatatcagcaagagcaacagagccagatattattaatgcaagtgtagaggcttatatagatggtgttaataggttattggagaataaataa

SEQ ID NO: 47 - protein, 516 aa, Leptospira interrogans
MTKVETRLEILDVTLRDGEQTRGVSFSTSEKLNIAKFLLQKLNVDRVEIASARVSKGELETVQKIMEWAATEQLTERIEILGFVDGNKTVDWIKDSGAKVLNLLTKGSLHHLEKQLGKTPKEFFTDVSFVIEYAIKSGLKINVYLEDWSNGFRNSPDYVKSLVEHLSKEHIERIFLPDTLGVLSPEETFQGVDSLIQKYPDIHFEFHGHNDYDLSVANSLQAIRAGVKGLHASINGLGERAGNTPLEALVTTIHDKSNSKTNINEIAITEASRLVEVFSGKRISANRPIVGEDVFTQTAGVHADGDKKGNLYANPILPERFGRKRSYALGKLAGKASISENVKQLGMVLSEVVLQKVLERVIELGDQNKLVTPEDLPFIIADVSGRTGEKVLTIKSCNIHSGIGIRPHAQIELEYQGKIHKEISEGDGGYDAFMNALTKITNRLGISIPKLIDYEVRIPPGGKTDALVETRITWNKSLDLEEDQTFKTMGVHPDQTVAAVHATEKMLNQILQPWQI

SEQ ID NO: 48 - DNA, 1551 bp, Leptospira interrogans
atgacaaaagtagaaactcgattggaaattttagacgtaactttgagagacggggagcagaccagaggggtcagtttttccacttccgaaaaactaaatatcgcaaaatttctattacaaaaactaaatgtagatcgggtagagattgcgtctgcaagagtttctaaaggggaattggaaacggtccaaaaaatcatggaatgggctgcaacagaacagcttacggaaagaatcgaaatcttaggttttgtagacgggaataaaaccgtagattggatcaaagatagtggggctaaggttttaaatcttttgactaagggatcgcttcatcatttagaaaaacaattaggcaaaactccgaaagaattctttacagacgtttcttttgtaatagaatacgcgatcaaaagcggacttaaaataaacgtatatttagaagattggtccaacggtttcagaaacagtccagattacgtcaaatcgctcgtagaacatctaagtaaagaacatatagaaagaatttttcttccagacacgttaggcgttctttcgccagaagagacgtttcaaggagtggattcactcattcaaaaatacccggatattcattttgaatttcacggacataacgactacgatctttccgtggcaaatagtcttcaagcgattcgtgccggagtcaaaggtcttcacgcttctataaatggtctcggagaaagagccggaaatactccgttggaagcactcgtaaccacgattcatgataagtctaactctaaaacgaacataaacgaaattgcaattacggaagcaagccgtcttgtagaagtattcagcggaaaaagaatttctgcaaatagaccgatcgtaggagaagacgtgtttactcagaccgcgggagtacacgcagacggagacaaaaaaggaaatttatacgcaaatcctattttaccggaaagatttggtaggaaaagaagttacgcgttaggcaaacttgcaggtaaggcgagtatctccgaaaatgtaaaacaactcggaatggttttaagtgaagtggttttacaaaaggttttagaaagggtgatcgaattaggagatcagaataaactagtgacacctgaagatcttccatttatcattgcggacgtttctggaagaaccggagaaaaggtacttacaatcaaatcttgtaatattcattccggaattggaattcgtcctcacgcacaaattgaattggaatatcagggaaagattcataaggaaatttctgaaggagacggagggtatgatgcgtttatgaatgcacttactaaaattacgaatcgcctcggtattagtattcctaaattgatagattacgaagtaaggattcctcctggtggaaaaacagatgcacttgtagaaactaggatcacctggaacaagtccttagatttagaagaggaccagactttcaaaacgatgggagttcatccggatcaaacggttgcagcggttcatgcaactgaaaagatgctcaatcaaattctacaaccatggcaaatctaa

SEQ ID NO: 49 - protein, 466 aa, Salmonella typhimurium
MAKTLYEKLFDAHVVFEAPNETPLLYIDRHLVHEVTSPQAFDGLRAHHRPVRQPGKTFATMDHNVSTQTKDINASGEMARIQMQELIKNCNEFGVELYDLNHPYQGIVHVMGPEQGVTLPGMTIVCGDSHTATHGAFGALAFGIGTSEVEHVLATQTLKQGRAKTMKIEVTGNAAPGITAKDIVLAIIGKTGSAGGTGHVVEFCGDAIRALSMEGRMTLCNMAIEMGAKAGLVAPDETTFNYVKGRLHAPKGRDFDEAVEYWKTLKTDDGATFDTVVALRAEEIAPQVTWGTNPGQVISVTDIIPDPASFSDPVERASAEKALAYMGLQPGVPLTDVAIDKVFIGSCTNSRIEDLRAAAEVAKGRKVAPGVQALVVPGSGPVKAQAEAEGLDKIFIEAGFEWRLPGCSMCLAMNNDRLNPGERCASTSNRNFEGRQGRGGRTHLVSPAMAAAAAVTGHFADIRSIK

SEQ ID NO: 50 - DNA, 1401 bp, Salmonella typhimurium
atggccaaaacgttatacgaaaaattatttgatgcccacgtggtctttgaggcgccaaacgaaacgccgctgctgtacatcgaccgccacctggtgcatgaagtcacctctccgcaggcgtttgacggtctgcgcgcgcaccatcgtccggtacgtcagccagggaaaaccttcgctacgatggatcacaacgtctcgacgcagactaaagacattaatgcttccggtgaaatggcgcgtatccagatgcaggagctgattaagaactgtaacgagttcggcgtcgagctgtatgacctgaatcacccatatcagggcatcgtccatgtgatggggccggaacagggcgtcaccctgccgggcatgaccatcgtctgcggcgactcccacaccgccacccacggcgcgtttggtgcgctggccttcggcatcggcacttctgaggtagaacatgtactggcgacgcaaaccctgaaacagggacgcgctaaaaccatgaagattgaagtcacgggcaacgccgcgccgggcattaccgccaaagacatcgtgctggcgatcatcggtaaaaccggtagcgccggcggcaccggacacgtggttgaattttgcggcgacgctatccgcgcgctgagtatggaaggccgcatgacgctgtgcaatatggcgattgagatgggcgccaaagccggtctggtcgccccggatgaaaccactttcaactacgtaaaagggcgtttgcacgcgccgaagggccgcgattttgacgaagccgtcgagtactggaaaacgctgaaaaccgatgacggcgcgacctttgatactgtcgtcgccctgcgagcagaagagatcgcgccgcaggtgacctggggcacgaatccgggccaggtgatttccgtcaccgacatcatccccgatcccgcctcctttagcgatccggttgagcgcgccagcgccgaaaaagcgctggcttatatgggcttacagccgggcgtaccgttaacggacgttgctatcgataaagtctttatcggctcttgtaccaattcacgcattgaagatttgcgcgcggcggcggaagtcgccaaagggcgcaaagttgcgccgggcgtgcaggcgctggtggtgccgggttcaggtccggtgaaagcgcaggcggaagcggaaggtctggacaagatctttatcgaagcaggatttgaatggcgcttaccgggctgttccatgtgcctggccatgaataacgaccgcctgaacccgggcgagcgctgcgcctccaccagcaaccgtaactttgaaggtcgtcagggccgcgggggtcgcacgcatttagtcagcccggcgatggccgccgctgccgccgttaccggccacttcgccgacattcgcagcatcaaataa

SEQ ID NO: 51 - protein, 201 aa, Salmonella typhimurium
MAEKFIQHTGLVVPLDAANVDTDAIIPKQFLQKVTRTGFGAHLFNDWRFLDEQGQQPNPAFVLNFPEYQGASILLARENFGCGSSREHAPWALTDYGFKVVIAPSFADIFYGNSFNNQLLPVKLSEEEVDELFALVQANPGIHFEVDLEAQVVKAGDKRYPFEIDAFRRHCMMNGLDSIGLTLQHEDAIAAYENKQPAFMR

SEQ ID NO: 52 - DNA, 606 bp, Salmonella typhimurium
atggcagagaaatttacccagcataccggcctggttgtcccactggatgccgccaacgtcgataccgatgcaattatccctaaacagtttttgcagaaggttacgcgcaccggttttggcgcccatctgtttaacgactggcgtttcctggacgaaaagggccaacagccaaatccggaattcgtgttgaactttccggaatatcaaggcgcgtcgatactgttggcgcgggaaaactttggctgcggctcgtcacgcgagcacgcgccgtgggcgttgaccgattacggctttaaagtggtgatcgcgccaagcttcgccgacatcttctacggcaacagtttcaataatcaactgctgccggtaaccctgagcgacgcacaggtcgatgagctgtttgccctggtgaaagccaatccgggcattaaatttgaagtggatctggaagcacaggtggtgaaagcaggcgataaaacctacagctttaaaatcgacgacttccgccgccactgcatgttgaacggtctggacagcattgggctgacgctgcagcacgaagacgcgattgccgcctacgaaaataaacaaccggcatttatgcggtaa

SEQ ID NO: 53 - protein, 363 aa, Shigella boydii
MSKNYHIAVLPGDGIGPEVMTQALKVLDAVRNRFAMRITTSHYDVGGAAIDNHGQPLPPATVEGCEQADAVLFGSVGGPKWEHLPPDQQPERGALLPLRKHFKLFSNLRPAKLYQGLEAFCPLRADIAANGFDILCVRELTGGIYFGQPKGREGSGQYEKAFDTEVYHRFEIERIARIAFESARKRRHKVTSIDKANVLQSSILWREIVNEIATEYPDVELAHMYIDNATMQLIKDPSQFDVLLCSNLFGDILSDECAMITGSMGMLPSASLNEQGFGLYEPAGGSAPDITGKNIANPIAQILSLALLLRYSLDADDAACAIERAINRALEEGIRTGDLARGAAAVSTDEMGDIIARYVAEGV

SEQ ID NO: 54 - DNA, 1092 bp, Shigella boydii
atgtcgaagaattaccatattgccgtattgccgggggacggtattggtccggaagtgatgacccaggcgctgaaagtgctggatgccgtgcgcaaccgctttgcgatgcgcatcaccaccagccattacgatgtaggcggcgcagccattgataaccacgggcaaccactgccgcctgcgacggttgaaggttgtgagcaagccgatgccgtgctgtttggctcggtaggcggtccgaaatgggaacatttaccaccagaccagcaaccagaacgcggcgcgctgttgcctttgcgtaagcacttcaaattattcagcaacctgcgtccggcaaaactgtatcaggggctggaagcattctgtccgctgcgtgctgacattgccgctaacggcttcgacatcctgtgcgtgcgcgaactgaccggcggcatctatttcggtcagccaaaaggccgcgaaggtagcggacagtatgaaaaagcgtttgataccgaggtgtatcaccgttttgagatcgaacgtatcgcccgcatcgcgtttgaatctgcccgcaagcgtcgccacaaagtcacctcaatcgacaaagccaacgtgctgcaatcctctattttatggcgggagatcgttaacgagatcgccacggaatacccggatgtcgaactggcgcatatgtacatcgacaacgccaccatgcagctgattaaagatccatcacagtttgacgtcctgctgtgctccaacctgtttggcgacattctgtctgacgagtgcgcaatgatcactggctcgatggggatgttgccttccgccagcctgaacgagcaaggttttggtctgtatgaaccggcaggcggctcagcaccagatatcacaggcaaaaacatcgccaacccgattgcgcaaattctgtcgctggcactgctgctgcgctacagcctggatgccgatgatgcggcttgcgccattgaacgcgccattaaccgcgcattagaagaaggcattcgcaccggggatttagcccgtggcgctgccgccgttagtaccgatgaaatgggcgatatcattgcccgctatgtggcagaaggggtgtaa

SEQ ID NO: 55 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 56 - DNA, 2652 bp, Escherichia coli
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaa

SEQ ID NO: 57 - protein, 1154 aa, Rhizobium etli
LPISKILVANRSEIAIRVFRAANELGIKTVAIWAEEDKLALHRFKADESYQVGRGPHLARDLGPIESYLSIDEVIRVAKLSGADAIHPGYGLLSESPEFVDACNKAGIIFIGPKADTMRQLGNKVAARNLAISVGVPVVPATEPLPDDMAEVAKMAAAIGYPVMLKASWGGGGRGMRVIRSEADLAKEVTEAKREAMAAFGKDEVYLEKLVERARHVESQILGDTHGNVVHLFERDCSVQRRNQKVVERAPAPYLSEAQRQELAAYSLKIAGATNYIGAGTVEYLMDADTGKFYFIEVNPRIQVEHTVTEVVTGIDIVKAQIHILDGAAIGTPQSGVPNQEDIRLNGHALQCRVTTEDPEHNFIPDYGRITAYRSASGFGIRLDGGTSYSGAIITRYYDPLLVKVTAWAPNPLEAISRMDRALREFRIRGVATNLTFLEAIIGHPKFRDNSYTTRFIDTTPELFQQVKRQDRATKLLTYLADVTVNGHPEAKDRPKPLENAARPVVPYANGNGVKDGTKQLLDTLGPKKFGEWMRNEKRVLLTDTTMRDGHQSLLATRMRTYDIARIAGTYSHALPNLLSLECWGGATFDVSMRFLTEDPWERLALIREGAPNLLLQMLLRGANGVGYTNYPDNVVKYFVRQAAKGGIDLFRVFDCLNWVENMRVSMDAIAEENKLCEAAICYTGDILNSARPKYDLKYYTNLAVELEKAGAHIIAVKDMAGLLKPAAAKVLFKALREATGLPIHFHTHDTSGIAAATVLAAVEAGVDAVDAAMDALSGNTSQPCLGSIVEALSGSERDPGLDPAWIRRISFYWEAVRNQYAAFESDLKGPASEVYLHEMPGGQFTNLKEQARSLGLETRWHQVAQAYADANQMFGDIVKVTPSSKVVGDMALMMVSQDLTVADVVSPDREVSFPESVVSMLKGDLGQPPSGWPEALQKKALKGEKPYTVRPGSLLKEADLDAERKVIEKKLEREVSDFEFASYLMYPKVFTDFALASDTYGPVSVLPTPAYFYGLADGEELFADIEKGKTLVIVNQAVSATDSQGMVTVFFELNGQPRRIKVPDRAHGATGAAVRRKAEPGNAAHVGAPMPGVISRVFVSSGQAVNAGDVLVSIEAMKMETAIHAEKDGTIAEVLVKAGDQIDAKDLLAVYGG

SEQ ID NO: 58 - DNA, 3465 bp, Rhizobium etli
ttgcccatatccaagatactcgttgccaatcgctctgaaatagccatccgcgtgttccgcgcggccaacgagcttggaataaaaacggtggcgatctgggcggaagaggacaagctggcgctgcaccgcttcaaggcggacgagagttatcaggtcggccgcggaccgcatcttgcccgcgacctcgggccgatcgaaagctatctgtcgatcgacgaggtgatccgcgtcgccaagctttccggtgccgacgccatccatccgggctacggcctcttgtcggaaagccccgaattcgtcgatgcctgcaacaaggccggcatcatcttcatcggcccgaaggccgatacgatgcgccagcttggcaacaaggtcgcagcgcgcaacctggcgatctcggtcggcgtaccggtcgtgccggcgaccgagccactgccggacgatatggccgaagtggcgaagatggcggcggcgatcggctatcccgtcatgctgaaggcatcctggggcggcggcggtcgcggcatgcgcgtcattcgttccgaggccgacctcgccaaggaagtgacggaagccaagcgcgaggcgatggcggccttcggcaaggacgaggtctatctcgaaaaactggtcgagcgcgcccgccacgtcgaaagccagatcctcggcgacacccacggcaatgtcgtgcatctcttcgagcgcgactgttccgttcagcgccgcaatcagaaggtcgtcgagcgcgcgcccgcaccctatctttcggaagcgcagcgccaggaactcgccgcctattcgctgaagatcgcaggggcgaccaactatatcggcgccggcaccgtcgaatatctgatggatgccgataccggcaaattttacttcatcgaagtcaatccgcgcatccaggtcgagcacacggtgaccgaagtcgtcaccggcatcgatatcgtcaaggcgcagatccacatcctggacggcgccgcgatcggcacgccgcaatccggcgtgccgaaccaggaagacatccgtctcaacggtcacgccctgcagtgccgcgtgacgacggaagatccggagcacaacttcattccggattacggccgcatcaccgcctatcgctcggcttccggcttcggcatccggcttgacggcggcacctcttattccggcgccatcatcacccgctattacgatccgctgctcgtcaaggtcacggcctgggcgccgaacccgctggaagccatttcccgcatggaccgggcgctgcgcgaattccgcatccgtggcgtcgccaccaacctgaccttcctcgaagcgatcatcggccatccgaaattccgcgacaacagctacaccacccgcttcatcgacacgacgccggagctcttccagcaggtcaagcgccaggaccgcgcgacgaagcttctgacctatctcgccgacgtcaccgtcaatggccatcccgaggccaaggacaggccgaagcccctcgagaatgccgccaggccggtggtgccctatgccaatggcaacggggtgaaggacggcaccaagcagctgctcgatacgctcggcccgaaaaaattcggcgaatggatgcgcaatgagaagcgcgtgcttctgaccgacaccacgatgcgcgacggccaccagtcgctgctcgcaacccgcatgcgtacctatgacatcgccaggatcgccggcacctattcgcatgcgctgccgaacctcttgtcgctcgaatgctggggcggcgccaccttcgacgtctcgatgcgcttcctcaccgaagatccgtgggagcggctggcgctgatccgagagggggcgccgaacctgctcctgcagatgctgctgcgcggcgccaatggcgtcggttacaccaactatcccgacaatgtcgtcaaatacttcgtccgccaggcggccaaaggcggcatcgatctcttccgcgtcttcgactgcctgaactgggtcgagaatatgcgggtgtcgatggatgcgattgccgaggagaacaagctctgcgaggcggcgatctgctacaccggcgatatcctcaattccgcccgcccgaaatacgacttgaaatattacaccaaccttgccgtcgagcttgagaaggccggcgcccatatcattgcggtcaaggatatggcgggccttctgaagccggctgctgccaaggttctgttcaaggcgctgcgtgaagcaaccggcctgccgatccatttccacacgcatgacacctcgggcattgcggcggcaacggttcttgccgccgtcgaagccggtgtcgatgccgtcgatgcggcgatggatgcgctctccggcaacacctcgcaaccctgtctcggctcgatcgtcgaggcgctctccggctccgagcgcgatcccggcctcgatccggcatggatccgccgcatctccttctattgggaagcggtgcgcaaccagtatgccgccttcgaaagcgacctcaagggaccggcatcggaagtctatctgcatgaaatgccgggcggccagttcaccaacctcaaggagcaggcccgctcgctggggctggaaacccgctggcaccaggtggcgcaggcctatgccgacgccaaccagatgttcggcgatatcgtcaaggtgacgccatcctccaaggtcgtcggcgacatggcgctgatgatggtctcccaggacctgaccgtcgccgatgtcgtcagccccgaccgcgaagtctccttcccggaatcggtcgtctcgatgctgaagggcgatctcggccagcctccgtctggatggccggaagcgctgcagaagaaagcattgaagggcgaaaagccctatacggtgcgccccggctcgctgctcaaggaagccgatctcgatgcggaacgcaaagtcatcgagaagaagcttgagcgcgaggtcagcgacttcgaattcgcttcctatctgatgtatccgaaggtcttcaccgactttgcgcttgcctccgatacctacggtccggtttcggtgctgccgacgcccgcctatttttacgggttggcggacggcgaggagctgttcgccgacatcgagaagggcaagacgctcgtcatcgtcaatcaggcggtgagcgccaccgacagccagggcatggtcactgtcttcttcgagctcaacggccagccgcgccgtatcaaggtgcccgatcgggcccacggggcgacgggagccgccgtgcgccgcaaggccgaacccggcaatgccgcccatgtcggtgcgccgatgccgggcgtcatcagccgtgtctttgtctcttcaggccaggccgtcaatgccggcgacgtgctcgtctccatcgaggccatgaagatggaaaccgcgatccatgcggaaaaggacggcaccattgccgaagtgctggtcaaggccggcgatcagatcgatgccaaggacctgctggcggtttacggcggatga

SEQ ID NO: 59 - protein, 1167 aa, Ralstonia eutropha
MDYAPIRSLLIANRSEIAIRVMRAAAEMNVRTVAIYSKEDRLALHRFKADESYLVGEGKKPLAAYLDIDDILRIARQAKVDAIHPGYGFLSENPDFAQAVIDAGIRWIGPSPEVMRKLGNKVAARNAAIDAGVPVMPATDPLPHDLDTCKRLAAGIGYPLMLKASWGGGGRGMRVLEREQDLEGALAAARREALAAFGNDEVYVEKLVRNARHVEVQVLGDTHGNLVHLYERDCTVQRRNQKVVERAPAPYLDDAGRAALCESALRLMRAVGYTHAGTVEFLMDADSGQFYFIEVNPRIQVEHTVTEMVTGIDIVKAQIRVTEGGHLGMTENTRNENGEIVVRAAGVPVQEAISLNGHALQCRITTEDPENGFLPDYGRLTAYRSAAGFGVRLDAGTAYGGAVITPYYDSLLVKVTTWAPTAPESIRRMDRALREFRIRGVASNLQFLENVINHPSFRSGDVTTRFIDLTPELLAFTKRLDRATKLLRYLGEVSVNGHPEMSGRTLPSLPLPAPVLPAFDTGGALPYGTRDRLRELGAEKFSRWMLEQKQVLLTDTTMRDAHQSLFATRMRTADMLPIAPFYARELSQLFSLECWGGATFDVALRFLKEDPWQRLEQLRERVPNVLFQMLLRGSNAVGYTNYADNVVRFFVRQAASAGVDVFRVFDSLNWVRNMRVAIDAVGESGALCEGAICYTGDLFDKSRAKYDLKYYVGIARELKQAGVHVLGIKDMAGICRPQAAAALVRALKEETGLPVHFHTHDTSGISAASALAAIEAGCDAVDGALDAMSGLTSQPNLSSIAAALAGSERDPGLSLERLHEASMYWEGVRRYYAPFESEIRAGTADVYRHEMPGGQYTNLREQARSLGIEHRWTEVSRAYAEVNQMFGDIVKVTPTSKVVGDLALMMVANDLSAADVCDPARETAFPESVVSLFKGELGFPPDGFPAELSRKVLRGEPPVPYRPGDQIPPVDLDAARAAAEAACEQPLDDRQLASYLMYPKQAGEYHAHVRNYSDTSVVPTPAYLYGLQPQEEVAIDIAAGKTLLVSLQGTHPDAEEGVIKVQFELNGQSRTTLVEQRSTTQAAAARHGRPVAEPDNPLHVAAPMPGSIVTVAVQPGQRVAAGTTLLALEAMKMETHIAAERDCEIAAVHVQQGDRVAAKDLLIELKG

SEQ ID NO: 60 - DNA, 3504 bp, Ralstonia eutropha
atggactacgcccctatccgctccctgctgattgccaaccgttccgagatcgcgatccgcgtgatgcgcgcggccgccgagatgaacgtgcgcacggtggcaatctattcgaaggaagaccggctcgcgctccatcgcttcaaggccgatgagagctacctggtcggcgagggcaagaagccactggcggcttacctcgacatcgacgatatcctgcgcattgccaggcaggcgaaggtcgacgccattcatccgggctatggcttcctttcagagaacccggacttcgcgcaggccgtgatcgacgcgggtatccgctggatcggcccgtcgcccgaggtcatgcgcaagcttggcaacaaggtggcggcgcgcaacgcggcgatcgacgcgggcgtgccggtgatgccggcaaccgatccgctgccgcatgacctggacacgtgcaagcgcctcgccgccggcatcggctatccgctgatgctcaaggcaagctggggcggcggcggacgcggcatgcgggtcctggaacgcgagcaggaccttgagggggcgctcgccgcggcgcggcgcgaggcgctggctgcgttcggcaacgacgaggtgtatgtcgagaagctggtgcgcaacgcgcgccatgtcgaagtgcaggtgctcggcgacacgcacggcaacctcgtgcatctctatgagcgcgactgtaccgtgcagcggcgcaaccagaaggtggtggagcgggcgcccgcgccatacctcgacgatgccggccgggccgcgctgtgcgaatcggccctgcggctgatgcgcgcggtcggctacacgcatgccggtacggtcgagttcctgatggatgccgactccggccagttctacttcatcgaggtcaatccgcgcatccaggtcgagcacacggtcacggagatggtcaccgggatcgatatcgtcaaggcgcagatccgcgtgaccgaaggcggccatctcggcatgaccgagaacacgcgcaatgagaacggcgagatcgtcgtgcgcgccgcgggcgtgccggtgcaggaagcgatttcgctcaacggtcacgcgctgcaatgccggatcaccaccgaggacccggagaacgggttcctgccggactacggccgcctcactgcctaccgcagcgcggccggcttcggcgtgcgcctggacgccggcaccgcctacggcggcgcggtgatcacgccgtactacgattcgctgctggtcaaggttaccacctgggcgccgaccgcgcccgaatcgatccggcgcatggaccgcgcgctgcgcgagttccgcatccgcggcgtcgcgtccaacctgcagttcctcgagaacgtcatcaaccatccctcgttccggtccggcgacgtcaccacgcgctttatcgacctgacgccggaactgctggcgttcaccaagcgcctggaccgcgccaccaagctgctgcgctacctgggcgaggtcagcgtcaacgggcacccggagatgagcggccgcacgctgccatcgctgccgctgcccgcaccggtgctgcccgccttcgacaccggcggcgcgctgccctacggtacgcgcgaccggctgcgcgagctgggcgcggagaagttctcgcgctggatgctggagcagaagcaggtgctgctgaccgataccaccatgcgcgacgcgcaccagtcgctgttcgccacgcgcatgcgcaccgccgacatgctgccgatcgcgccgttctatgcgcgcgaactgtcgcagctgttctcgctggagtgctggggcggcgccaccttcgacgtggcgctgcgcttcctcaaggaagacccgtggcagcgccttgagcaactgcgcgagcgcgttcccaacgtgctgttccagatgctgctgcgcggctccaacgcggttggctacaccaattatgcggacaacgtggtgcgcttcttcgtgcgccaggcggccagcgccggcgtggatgtgttccgcgtgttcgattcactgaactgggtgcgcaacatgcgcgtggcgatcgatgctgtcggcgagagcggcgcgctgtgcgaaggcgcgatctgctataccggcgacctgttcgacaagtcgcgcgccaaatacgacctgaagtactacgtaggcatcgcgcgcgagctgaagcaggccggcgtgcacgtgctgggcatcaaggacatggccggcatctgccgtccgcaggccgcggcggcactggtcagggcgctcaaggaagagaccgggctgccggtgcatttccatacccacgataccagcggcatctcggccgcttcggcgctggccgcgatcgaggccggctgcgatgcggtcgacggcgcgctcgacgccatgagcgggctgacctcgcaacccaacctgtcgagcatcgccgcggccctggccggcagcgagcgcgatcccggcctcagcctggagcgcctgcacgaggcgtcgatgtactgggaaggggtgcgccgctactacgcgccgttcgaatccgaaatccgcgccggcaccgccgacgtgtaccgccacgagatgcccggcggccagtacaccaacctgcgcgagcaggcgcgctcgctcggcatcgagcatcgctggaccgaggtgtcgcgggcctatgccgaggtcaaccagatgtttggcgacatcgtcaaggtgacgccgacgtccaaggtggtcggcgacctggccttgatgatggtggccaacgacctgagcgccgccgatgtgtgcgatcccgccagggagactgccttccctgaatcggtggtgtcgctgttcaagggcgagctgggctttccgccggacggcttccccgcggaactgtcgcgcaaggtgctgcgcggcgagccgcccgtgccgtaccggcccggcgaccagatcccgccggtcgacctcgacgcggcgcgcgccgcggccgaagcggcgtgcgagcagccgctcgacgaccgccagctggcttcgtacctgatgtacccgaagcaggccggcgagtaccacgcgcatgtgcgcaactacagcgacacctcggtggtacccacgccggcatacctgtacggcctgcagccgcaggaagaagtggcgatcgacatcgctgccggcaagaccctgctggtctcgctgcaaggcacgcaccccgatgccgaagagggtgtcatcaaggtccagttcgagctgaacgggcagtcgcgcaccacgctggtcgagcagcgcagcaccacgcaagcggcggcagcgcgccatggccgtccggttgccgaacccgacaatccgctgcatgtcgccgcgcccatgccgggctcgatcgtgacggtggcggtgcagccggggcagcgcgtggccgcgggcacgacgctgctggcgctggaggcgatgaagatggaaacccatatcgcggcggagcgggactgcgagatcgccgcagtccatgttcagcagggggatcgcgtggcggcgaaggatctgctgatcgaactgaagggctga

SEQ ID NO: 61 - protein, 820 aa, Escherichia coli
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARIFVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 62 - DNA, 2463 bp, Escherichia coli
atgcgtgtgctgaagttcggtggtacgagcgtggctaatgctgaacgttttctgcgtgttgctgacatcctggaatcaaatgcccgtcagggtcaagttgcaaccgtcctgagcgcaccggcaaaaattacgaatcatctggtggccatgattgaaaagaccatctcgggtcaggatgcactgccgaacattagcgacgctgaacgcatctttgcggaactgctgaccggcctggcggcggcgcagccgggtttcccgctggctcaactgaaaacgtttgttgatcaggaatttgcgcaaattaagcatgtcctgcacggcatctccctgctgggtcaatgcccggattcaattaatgctgcgctgatctgtcgcggcgaaaaaatgtctattgctatcatggcgggcgtgctggaagcccgtggtcataacgtcaccgtgattgatccggtggaaaaactgctggctgttggtcactatctggaaagcaccgtggatattgcagaatctacgcgtcgcattgccgcaagtcgtatcccggcggaccatatggtgctgatggctggttttaccgcgggcaatgaaaaaggtgaactggtggttctgggtcgcaacggctcagattattcggctgcggtgctggccgcatgcctgcgtgcagactgctgtgaaatttggaccgatgtggacggcgtttacacgtgtgatccgcgtcaggttccggacgcacgtctgctgaaatccatgtcatatcaagaagctatggaactgagctactttggtgcgaaggtgctgcacccgcgtaccattacgccgatcgcgcagttccaaattccgtgcctgatcaaaaacaccggtaatccgcaggctccgggcacgctgattggtgcgtctcgtgatgaagacgaactgccggtcaaaggtatcagtaatctgaacaatatggccatgtttagcgtgagcggcccgggtatgaagggtatggtcggtatggctgcgcgcgtgtttgcagcaatgtctcgtgcgcgcattttcgtcgtgctgatcacccagagcagcagcgaatattctattagtttttgcgttccgcagagtgattgtgtccgtgccgaacgcgcaatgcaggaagaattttacctggaactgaaagaaggcctgctggaaccgctggccgttaccgaacgcctggcaattatctccgttgtcggcgatggtatgcgtacgctgcgcggtatctcagcgaaatttttcgctgcgctggctcgcgcgaacattaatatcgtggccattgcacagggctcctcagaacgttccatctcagtggttgtcaacaatgatgacgccaccacgggtgttcgtgtcacccatcagatgctgtttaatacggatcaagttattgaagtgttcgttatcggtgtcggcggtgtgggcggtgcgctgctggaacaactgaaacgccagcaatcgtggctgaaaaacaagcatattgatctgcgtgtttgcggcgtcgccaatagcaaggcactgctgaccaacgtgcacggtctgaacctggaaaattggcaggaagaactggctcaagcgaaagaaccgtttaatctgggccgtctgattcgcctggttaaggaatatcacctgctgaacccggtcatcgtggattgtaccagcagccaggccgtcgcagatcaatacgcagactttctgcgcgaaggtttccatgtggttaccccgaataaaaaggcgaacacgtctagtatggattattaccaccaactgcgttatgccgcagaaaaatctcgtcgcaagtttctgtacgacaccaatgtgggcgcgggtctgccggttattgaaaacctgcaaaatctgctgaatgccggcgatgaactgatgaaattcagtggcattctgtcgggtagcctgtcttatatctttggcaagctggatgagggtatgagtttctccgaagctaccacgctggcgcgtgaaatgggctacaccgaaccggacccgcgtgatgacctgtccggtatggacgttgcccgtaaactgctgattctggcacgtgaaacgggccgcgaactggaactggccgatattgaaatcgaaccggtgctgccggcggaatttaatgcagaaggtgacgttgctgcgttcatggcgaacctgagccaactggatgacctgtttgccgcacgtgtggctaaagcgcgcgatgaaggcaaggtcctgcgctatgtgggcaatattgatgaagacggtgtgtgtcgtgttaaaatcgcggaagtcgatggcaacgacccgctgtttaaagtgaagaatggtgaaaacgccctggcattctattcccattattaccagccgctgccgctggttctgcgcggttacggtgccggcaacgatgttaccgctgcgggcgtcttcgcagacctgctgcgtacgctgtcatggaaactgggtgtgtaa

SEQ ID NO: 63 - protein, 449 aa, Escherichia coli
MSEIVVSKFGGTSVADFDAMNRSADIVLSDANVRLVVLSASAGITNLLVALAEGLEPGERFEKLDAIRNIQFAILERLRYPNVIREEIERLLENITVLAEAAALATSPALTDELVSHGELMSTLLFVEILRERDVQAQWFDVRKVMRTNDRFGRAEPDIAALAELAALQLLPRLNEGLVITQGFIGSENKGRTTTLGRGGSDYTAALLAEALHASRVDIWTDVPGIYTTDPRVVSAAKRIDEIAFAEAAEMATFGAKVLHPATLLPAVRSDIPVFVGSSKDPRAGGTLVCNKTENPPLFRALALRRNQTLLTLHSLNMLHSRGFLAEVFGILARHNISVDLITTSEVSVALILDTTGSTSTGDTLLTQSLLMELSALCRVEVEEGLALVALIGNDLSKACGVGKEVFGVLEPFNIRMICYGASSHNLCFLVPGEDAEQVVQKLHSNLFE

SEQ ID NO: 64 - DNA, 1350 bp, Escherichia coli
atgtctgaaattgttgtctccaaatttggcggtaccagcgtagctgattttgacgccatgaaccgcagcgctgatattgtgctttctgatgccaacgtgcgtttagttgtcctctcggcttctgctggtatcactaatctgctggtcgctttagctgaaggactggaacctggcgagcgattcgaaaaactcgacgctatccgcaacatccagtttgccattctggaacgtctgcgttacccgaacgttatccgtgaagagattgaacgtctgctggagaacattactgttctggcagaagcggcggcgctggcaacgtctccggcgctgacagatgagctggtcagccacggcgagctgatgtcgaccctgctgtttgttgagatcctgcgcgaacgcgatgttcaggcacagtggtttgatgtacgtaaagtgatgcgtaccaacgaccgatttggtcgtgcagagccagatatagccgcgctggcggaactggccgcgctgcagctgctcccacgtctcaatgaaggcttagtgatcacccagggatttatcggtagcgaaaataaaggtcgtacaacgacgcttggccgtggaggcagcgattatacggcagccttgctggcggaggctttacacgcatctcgtgttgatatctggaccgacgtcccgggcatctacaccaccgatccacgcgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaatccttgataccaccggttcaacctccactggcgatacgttgctgacgcaatctctgctgatggagctttccgcactgtgtcgggtggaggtggaagaaggtctggcgctggtcgcgttgattggcaatgacctgtcaaaagcctgcggcgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtaa

SEQ ID NO: 65 - protein, 810 aa, Escherichia coli
MSVIAQAGAKGRQLHKFGGSSLADVKCYLRVAGIMAEYSQPDDMMVVSAAGSTTNQLINWLKLSQTDRLSAHQVQQTLRRYQCDLISGLLPAEEADSLISAFVSDLERLAALLDSGINDAVYAEVVGHGEVWSARLMSAVLNQQGLPAAWLDAREFLRAERAAQPQVDEGLSYPLLQQLLVQHPGKRLVVTGFISRNNAGETVLLGRNGSDYSATQIGALAGVSRVTIWSDVAGVYSADPRKVKDACLLPLLRLDEASELARLAAPVLHARTLQPVSGSEIDLQLRCSYTPDQGSTRIERVLASGTGARIVTSHDDVCLIEFQVPASQDFKLAHKEIDQILKRAQVRPLAVGVHNDRQLLQFCYTSEVADSALKILDEAGLPGELRLRQGLALVAMVGAGVTRNPLHCHRFWQQLKGQPVEFTWQSDDGISLVAVLRTGPTESLIQGLHQSVFRAEKRIGLVLFGKGNIGSRWLELFAREQSTLSARTGFEFVLAGVVDSRRSLLSYDGLDASRALAFFNDEAVEQDEESLFLWMRAHPYDDLVVLDVTASQQLADQYLDFASHGFHVISANKLAGASDSNKYRQIHDAFEKTGRHWLYNATVGAGLPINHTVRDLIDSGDTILSISGIFSGTLSWLFLQFDGSVPFTELVDQAWQQGLTEPDPRDDLSGKDVMRKLVILAREAGYNIEPDQVRVESLVPAHCEGGSIDHFFENGDELNEQMVQRLEAAREMGLVLRYVARFDANGKARVGVEAVREDHPLASLLPCDNVFAIESRWYRDNPLVIRGPGAGRDVTAGAIQSDINRLAQLL

SEQ ID NO: 66 - DNA, 2433 bp, Escherichia coli
atgagtgtgattgcgcaggcaggggcgaaaggtcgtcagctgcataaatttggtggcagtagtctggctgatgtgaagtgttatttgcgtgtcgcgggcattatggcggagtactctcagcctgacgatatgatggtggtttccgccgccggtagcaccactaaccagttgattaactggttgaaactaagccagaccgatcgtctctctgcgcatcaggttcaacaaacgctgcgtcgctatcagtgcgatctgattagcggtctgctacccgctgaagaagccgatagcctcattagcgcttttgtcagcgaccttgagcgcctggcggcgctgctcgacagcggtattaacgacgcagtgtatgcggaagtggtgggccacggggaagtatggtcggcacgtctgatgtctgcggtacttaatcaacaagggctgccagcggcctggcttgatgcccgcgagtttttacgcgctgaacgcgccgcacaaccgcaggttgatgaagggctttcttacccgttgctgcaacagctgctggtgcaacatccgggcaaacgtctggtggtgaccggatttatcagccgcaacaacgccggtgaaacggtgctgctggggcgtaacggttccgactattccgcgacacaaatcggtgcgctggcgggtgtttctcgcgtaaccatctggagcgacgtcgccggggtatacagtgccgacccgcgtaaagtgaaagatgcctgcctgctgccgttgctgcgtctggatgaggccagcgaactggcgcgcctggcggctcccgttcttcacgcccgtactttacagccggtttctggcagcgaaatcgacctgcaactgcgctgtagctacacgccggatcaaggttccacgcgcattgaacgcgtgctggcctccggtactggtgcgcgtattgtcaccagccacgatgatgtctgtttgattgagtttcaggtgcccgccagtcaggatttcaaactggcgcataaagagatcgaccaaatcctgaaacgcgcgcaggtacgcccgctggcggttggcgtacataacgatcgccagttgctgcaattttgctacacctcagaagtggccgacagtgcgctgaaaatcctcgacgaagcgggattacctggcgaactgcgcctgcgtcaggggctggcgctggtggcgatggtcggtgcaggcgtcacccgtaacccgctgcattgccaccgcttctggcagcaactgaaaggccagccggtcgaatttacctggcagtccgatgacggcatcagcctggtggcagtactgcgcaccggcccgaccgaaagcctgattcaggggctgcatcagtccgtcttccgcgcagaaaaacgcatcggcctggtattgttcggtaagggcaatatcggttcccgttggctggaactgttcgcccgtgagcagagcacgctttcggcacgtaccggctttgagtttgtgctggcaggtgtggtggacagccgccgcagcctgttgagctatgacgggctggacgccagccgcgcgttagccttcttcaacgatgaagcggttgagcaggatgaagagtcgttgttcctgtggatgcgcgcccatccgtatgatgatttagtggtgctggacgttaccgccagccagcagcttgctgatcagtatcttgatttcgccagccacggtttccacgttatcagcgccaacaaactggcgggagccagcgacagcaataaatatcgccagatccacgacgccttcgaaaaaaccgggcgtcactggctgtacaatgccaccgtcggtgcgggcttgccgatcaaccacaccgtgcgcgatctgatcgacagcggcgatactattttgtcgatcagcgggatcttctccggcacgctctcctggctgttcctgcaattcgacggtagcgtgccgtttaccgagctggtggatcaggcgtggcagcagggcttaaccgaacctgacccgcgtgacgatctctctggcaaagacgtgatgcgcaagctggtgattctggcgcgtgaagcaggttacaacatcgaaccggatcaggtacgtgtggaatcgctggtgcctgctcattgcgaaggcggcagcatcgaccatttctttgaaaatggcgatgaactgaacgagcagatggtgcaacggctggaagcggcccgcgaaatggggctggtgctgcgctacgtggcgcgtttcgatgccaacggtaaagcgcgtgtaggcgtggaagcggtgcgtgaagatcatccgttggcatcactgctgccgtgcgataacgtctttgccatcgaaagccgctggtatcgcgataaccctctggtgatccgcggacctggcgctgggcgcgacgtcaccgccggggcgattcagtcggatatcaaccggctggcacagttgttgtaa

SEQ ID NO: 67 - protein, 420 aa, Escherichia coli
MPHSLFSTDTDLTAENLLRLPAEFGCPVWVYDAQIIRRQIAALKQFDVVRFAQKACSNIHILRLMREQGVKVDSVSLGEIERALAAGYNPQTHPDDIVFTADVIDQATLERVSELQIPVNAGSVDMLDQLGQVSPGHRVWLRVNPGFGHGHSQKTNTGGENSKHGIWYTDLPAALDVIQRHHLQLVGIHMHIGSGVDYAHLEQVCGAMVRQVIEFGQDLQAISAGGGLSVPYQQGEEAVDTEHYYGLWNAAREQIARHLGHPVKLEIEPGRFLVAQSGVLITQVRSVKQMGSRHFVLVDAGFNDLMRPAMYGSYHHISALAADGRSLEHAPTVETVVAGPLCESGDVFTQQEGGNVETRALPEVKAGDYLVLHDTGAYGASMSSNYNSRPLLPEVLFDNGQARLIRRRQTIEELLALELL

SEQ ID NO: 68 - DNA, 1263 bp, Escherichia coli
atgccacattcactgttcagcaccgataccgatctcaccgccgaaaatctgctgcgtttgcccgctgaatttggctgcccggtgtgggtctacgatgcgcaaattattcgtcggcagattgcagcgctgaaacagtttgatgtggtgcgctttgcacagaaagcctgttccaatattcatattttgcgcttaatgcgtgagcagggcgtgaaagtggattccgtctcgttaggcgaaatagagcgtgcgttggcggcgggttacaatccgcaaacgcaccccgatgatattgtttttacggcagatgttatcgatcaggcgacgcttgaacgcgtcagtgaattgcaaattccggtgaatgcgggttctgttgatatgctcgaccaactgggccaggtttcgccagggcatcgggtatggctgcgcgttaatccggggtttggtcacggacatagccaaaaaaccaataccggtggcgaaaacagcaagcacggtatctggtacaccgatctgcccgccgcactggacgtgatacaacgtcatcatctgcagctggtcggcattcacatgcacattggttctggcgttgattatgcccatctggaacaggtgtgtggtgctatggtgcgtcaggtcatcgaattcggtcaggatttacaggctatttctgcgggcggtgggctttctgttccttatcaacagggtgaagaggcggttgataccgaacattattatggtctgtggaatgccgcgcgtgagcaaatcgcccgccatttgggccaccctgtgaaactggaaattgaaccgggtcgcttcctggtagcgcagtctggcgtattaattactcaggtgcggagcgtcaaacaaatggggagccgccactttgtgctggttgatgccgggttcaacgatctgatgcgcccggcaatgtacggtagttaccaccatatcagtgccctggcagctgatggtcgttctctggaacacgcgccaacggtggaaaccgtcgtcgccggaccgttatgtgaatcgggcgatgtctttacccagcaggaagggggaaatgttgaaacccgcgccttgccggaagtgaaggcaggtgattatctggtactgcatgatacaggggcatatggcgcatcaatgtcatccaactacaatagccgtccgctgttaccagaagttctgtttgataatggtcaggcgcggttgattcgccgtcgccagaccatcgaagaattactggcgctggaattgctttaa

SEQ ID NO: 69 - protein, 292 aa, Escherichia coli
MFTGSIVAIVTPMDEKGNVCRASLKKLIDYHVASGTSAIVSVGTTGESATLNHDEHADVVMMTLDLADGRIPVIAGTGANATAEAISLTQRFNDSGIVGCLTVTPYYNRPSQEGLYQHFKAIAEHTDLPQILYNVPSRTGCDLLPETVGRLAKVKNIIGIKEATGNLTRVNQIKELVSDDFVLLSGDDASALDFMQLGGHGVISVTANVAARDMAQMCKLAAEGHFAEARVINQRLMPLHNKLFVEPNPIPVKWACKELGLVATDTLRLPMTPITDSGRETVRAALKHAGLL

SEQ ID NO: 70 - DNA, 879 bp, Escherichia coli
atgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattgggcggtcatggggttatttccgttacggctaacgtcgcagcgcgtgatatggcccagatgtgcaaactggcagcagaagggcattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggtcgtgagacggtcagagcggcgcttaagcatgccggtttgctgtaa

SEQ ID NO: 71 - protein, 427 aa, Escherichia coli
MPIRVPDELPAVNFLREENVFVMTDTSRASGQEIRPLKVLILNLMPKKIETENQFLRLLSNSPLQVDIQLLRIDSRESRNTPAEHLNNFYCNFEDIQDQNFDGLIVTGAPLGLVEFNDVAYWPQIAALKQFDVVLEWSKDHILRLMREQGVTSTLFVCWAVQAALNILYGIPKQTRTEKLSGVYEHHILHPHALLTRGFDDSFLAGYNPQTHSRYADFPAAGSVDMLIRDYTDQLEILAETEEGDAYLFASKDKRIAFVTGHPEYDAQKTNTGGENSKHGIWYTDLPAALDVIQEFFRDVEAGLDPDVPYNYFPHNDPQNTPRASVPYQQGEEAVDTEHYYGLWNAAREQIARHLGHPVKLEIEPGRFLVAQSGVLITQVRSHGNLVDAGFNDLFTNWLNYYVYQITPYDLRHMNSRPTLLPEVLFD

SEQ ID NO: 72 - DNA, 930 bp, Eshcerichia coli
atgccgattcgtgtgccggacgagctacccgccgtcaatttcttgcgtgaagaaaacgtctttgtgatgacaacttctcgtgcgtctggtcaggaaattcgtccacttaaggttctgatccttaacctgatgccgaagaagattgaaactgaaaatcagtttctgcgcctgctttcaaactcacctttgcaggtcgatattcagctgttgcgcatcgattcccgtgaatcgcgcaacacgcccgcagagcatctgaacaacttctactgtaactttgaagatattcaggatcagaactttgacggtttgattgtaactggtgcgccgctgggcctggtggagtttaatgatgtcgcttactggccgcagatcaaacaggtgctggagtggtcgaaagatcacgtcacctcgacgctgtttgtctgctgggcggtacaggccgcgctcaatatcctctacggcattcctaagcaaactcgcaccgaaaaactctctggcgtttacgagcatcatattctccatcctcatgcgcttctgacgcgtggctttgatgattcattcctggcaccgcattcgcgctatgctgactttccggcagcgttgattcgtgattacaccgatctggaaattctggcagagacggaagaaggggatgcatatctgtttgccagtaaagataagcgcattgcctttgtgacgggccatcccgaatatgatgcgcaaacgctggcgcaggaatttttccgcgatgtggaagccggactagacccggatgtaccgtataactatttcccgcacaatgatccgcaaaatacaccgcgagcgagctggcgtagtcacggtaatttactgtttaccaactggctcaactattacgtctaccagatcacgccatacgatctacggcacatgaatccaacgctggattaa

SEQ ID NO: 73 - protein, 377 aa, Escherichia coli
MFTGSIVKVYAIVTPMDEKGNVCRASSANMSVGFDVLGAAVTPVDGALLGTTGESATLNHDEHADVVMMTVEADGRIPVIAETFSLNNLGRFADKLPSEPRENIVYQCWERFNDSGIVGCLTVTPYYNRPSQEGLGKQIPVAMTLEKNMPIGSGLGSSACSVVAALMAMNEHCGKPLNDTRLLALMGELAAEGHFAEARVISGSIHYDNVAPCFLGGMQLMIEENDIISQQVPGFDEWLWVLAYPGIKVSTAEARAILPAQYRRQDCIAHGRHLAGFIHACYSRQPELAAKLMKDVIAEPYRERLLPGFRQARQAVAEIGAVASGISGSGPTLFALCDKPETAQRVADWLGKNYLQNQEGFVHICRLDTAGARVLEN

SEQ ID NO: 74 - DNA, 933 bp, Escherichia coli
atggttaaagtttatgccccggcttccagtgccaatatgagcgtcgggtttgatgtgctcggggcggcggtgacacctgttgatggtgcattgctcggagatgtagtcacggttgaggcggcagagacattcagtctcaacaacctcggacgctttgccgataagctgccgtcagaaccacgggaaaatatcgtttatcagtgctgggagcgtttttgccaggaactgggtaagcaaattccagtggcgatgaccctggaaaagaatatgccgatcggttcgggcttaggctccagtgcctgttcggtggtcgcggcgctgatggcgatgaatgaacactgcggcaagccgcttaatgacactcgtttgctggctttgatgggcgagctggaaggccgtatctccggcagcattcattacgacaacgtggcaccgtgttttctcggtggtatgcagttgatgatcgaagaaaacgacatcatcagccagcaagtgccagggtttgatgagtggctgtgggtgctggcgtatccggggattaaagtctcgacggcagaagccagggctattttaccggcgcagtatcgccgccaggattgcattgcgcacgggcgacatctggcaggcttcattcacgcctgctattcccgtcagcctgagcttgccgcgaagctgatgaaagatgttatcgctgaaccctaccgtgaacggttactgccaggcttccggcaggcgcggcaggcggtcgcggaaatcggcgcggtagcgagcggtatctccggctccggcccgaccttgttcgctctgtgtgacaagccggaaaccgcccagcgcgttgccgactggttgggtaagaactacctgcaaaatcaggaaggttttgttcatatttgccggctggatacggcgggcgcacgagtactggaaaactaa

SEQ ID NO: 75 - protein, 428 aa, Escherichia coli
MKLYNLKDHNEQVSFAQAVTQGLGKNQGLFFPHDLPEFSLTEIDEMLKLDFVTRSAKILSAFIGDEIPQEILEERVRAAFAFPAPVANVESDVGCLELFHGPTLAFKDFGGRFMAQMLTHIAGDKPVTILTATSGDTGAAVAHAFYGLPNVKVVILYPRGKISPLQEKLFCTLGGNIETVAIDGDFDACQALVKQAFDDEELKVALGLNSANSINISRLLAQICYYFEAVAQLPQETRNQLVVSVPSGNFGDLTAGLLAKSLGLPVKRFIAATNVNDTVPRFLHDGQWSPKATQATLSNAMDVSQPNNWPRVEELFRRKIWQLKELGYAAVDDETTQQTMRELKELGYTSEPHAAVAYRALRDQLNPGEYGLFLGTAHPAKFKESVEAILGETLDLPKELAERADLPLLSHNLPADFAALRKLMMNHQ

SEQ ID NO: 76 - DNA, 1287 bp, Escherichia coli
atgaaactctacaatctgaaagatcacaacgagcaggtcagctttgcgcaagccgtaacccaggggttgggcaaaaatcaggggctgttttttccgcacgacctgccggaattcagcctgactgaaattgatgagatgctgaagctggattttgtcacccgcagtgcgaagatcctctcggcgtttattggtgatgaaatcccacaggaaatcctggaagagcgcgtgcgcgcggcgtttgccttcccggctccggtcgccaatgttgaaagcgatgtcggttgtctggaattgttccacgggccaacgctggcatttaaagatttcggcggtcgctttatggcacaaatgctgacccatattgcgggtgataagccagtgaccattctgaccgcgacctccggtgataccggagcggcagtggctcatgctttctacggtttaccgaatgtgaaagtggttatcctctatccacgaggcaaaatcagtccactgcaagaaaaactgttctgtacattgggcggcaatatcgaaactgttgccatcgacggcgatttcgatgcctgtcaggcgctggtgaagcaggcgtttgatgatgaagaactgaaagtggcgctagggttaaactcggctaactcgattaacatcagccgtttgctggcgcagatttgctactactttgaagctgttgcgcagctgccgcaggagacgcgcaaccagctggttgtctcggtgccaagcggaaacttcggcgatttgacggcgggtctgctggcgaagtcactcggtctgccggtgaaacgttttattgctgcgaccaacgtgaacgataccgtgccacgtttcctgcacgacggtcagtggtcacccaaagcgactcaggcgacgttatccaacgcgatggacgtgagtcagccgaacaactggccgcgtgtggaagagttgttccgccgcaaaatctggcaactgaaagagctgggttatgcagccgtggatgatgaaaccacgcaacagacaatgcgtgagttaaaagaactgggctacacttcggagccgcacgctgccgtagcttatcgtgcgctgcgtgatcagttgaatccaggcgaatatggcttgttcctcggcaccgcgcatccggcgaaatttaaagagagcgtggaagcgattctcggtgaaacgttggatctgccaaaagagctggcagaacgtgctgatttacccttgctttcacataatctgcccgccgattttgctgcgttgcgtaaattgatgatgaatcatcagtaa

SEQ ID NO: 77 - DNA, 37 bp, Artificial
  PCR primers to amplify the threonine deaminase gene from E. coli
gtgccatggctcatattacatacgatctgccggttgc

SEQ ID NO: 78 - DNA, 41 bp, Artificial
  PCR primers to amplify the threonine deaminase gene from E. coli
gatcgaattcatccttaggcgtcaacgaaaccggtgatttg

SEQ ID NO: 79 - DNA, 1209 bp, Methanococcus jannaschii
tcatgatggtgcgcatttttgataccacgctgcgtgacggtgaacagacgccgggcgttagcctgacgccgaacgataaactggaaattgccaaaaaactggatgaactgggcgttgacgtcatcgaagccggtagcgcagtgacctctaaaggcgaacgcgaaggtattaaactgatcacgaaagaaggcctgaatgccgaaatttgctctttcgttcgtgcactgccggtcgatattgacgcggccctggaatgtgatgttgacagcgtccatctggtggttccgacctctccgatccacatgaaatataaactgcgtaaaaccgaagatgaagtgctggttacggctctgaaagcggttgaatacgccaaagaacagggtctgattgtcgaactgtcagccgaagatgcaacgcgctcggacgtgaactttctgatcaaactgttcaatgaaggcgaaaaagttggtgcagatcgtgtctgcgtgtgtgacaccgttggcgtcctgacgccgcagaaatcacaagaactgttcaagaaaattaccgaaaacgtgaatctgccggtgtcggttcattgccacaacgatttcggtatggcgaccgcaaatgcgtgcagcgcggtgctgggcggtgcggttcaatgtcatgtcacggtgaacggcatcggtgaacgcgctggcaatgcgagtctggaagaagtcgtggcagcttccaaaattctgtatggttacgataccaaaatcaaaatggaaaaactgtacgaagtcagtcgcattgtgtcccgtctgatgaaactgccggtcccgccgaacaaagctatcgtgggcgataatgcttttgcgcatgaagcgggcattcacgtggacggtctgatcaaaaacaccgaaacgtatgaaccgattaaaccggaaatggttggcaatcgtcgccgtattatcctgggcaaacactctggtcgtaaagcgctgaaatacaaactggatctgatgggtattaacgttagtgacgaacaactgaacaaaatctatgaacgtgtgaaagaatttggcgatctgggtaaatacattagcgatgccgacctgctggcaatcgtgcgtgaagttaccggtaaactgtgatgtcgaagaattaccatattgccgtattgccgggggacggtattggtccggagcggccgcttaattaagtttaaactctagagaattc

SEQ ID NO: 80 - protein, 372 aa, Methanococcus jannaschii
MMVRIFDTTLRDGEQTPGVSLTPNDKLEIAKKLDELGVDVIEAGSAVTSKGEREGIKLITKEGLNAEICSFVRALPVDIDAALECDVDSVHLVVPTSPIHMKYKLRKTEDEVLVTALKAVEYAKEQGLIVELSAEDATRSDVNFLIKLFNEGEKVGADRVCVCDTVGVLTPQKSQELFKKITENVNLPVSVHCHNDFGMATANACSAVLGGAVQCHVTVNGIGERAGNASLEEVVAASKILYGYDTKIKMEKLYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKL

SEQ ID NO: 81 - DNA, 25 bp, Artificial
  PCR primers to amplify the three gene leuBCD complex from E. coli
ttggtccggaagtgatgacccaggc

SEQ ID NO: 82 - DNA, 43 bp, Artificial
  PCR primers to amplify the three gene leuBCD complex from E. coli
tatgtgcggccgcttaattcataaacgcaggttgttttgcttc

SEQ ID NO: 83 - DNA, 3081 bp, Escherichia coli
ttggtccggaagtgatgacccaggcgctgaaagtgctggatgccgtgcgcaaccgctttgcgatgcgcatcaccaccagccattacgatgtaggcggcgcagccattgataaccacgggcaaccactgccgcctgcgacggttgaaggttgtgagcaagccgatgccgtgctgtttggctcggtaggcggcccgaagtgggaacatttaccaccagaccagcaaccagaacgcggcgcgctgctgcctctgcgtaagcacttcaaattattcagcaacctgcgcccggcaaaactgtatcaggggctggaagcattctgtccgctgcgtgcagacattgccgcaaacggcttcgacatcctgtgtgtgcgcgaactgaccggcggcatctatttcggtcagccaaaaggccgcgaaggtagcggacaatatgaaaaagcctttgataccgaggtgtatcaccgttttgagatcgaacgtatcgcccgcatcgcgtttgaatctgctcgcaagcgtcgccacaaagtgacgtcgatcgataaagccaacgtgctgcaatcctctattttatggcgggagatcgttaacgagatcgccacggaatacccggatgtcgaactggcgcatatgtacatcgacaacgccaccatgcagctgattaaagatccatcacagtttgacgttctgctgtgctccaacctgtttggcgacattctgtctgacgagtgcgcaatgatcactggctcgatggggatgttgccttccgccagcctgaacgagcaaggttttggactgtatgaaccggcgggcggctcggcaccagatatcgcaggcaaaaacatcgccaacccgattgcacaaatcctttcgctggcactgctgctgcgttacagcctggatgccgatgatgcggcttgcgccattgaacgcgccattaaccgcgcattagaagaaggcattcgcaccggggatttagcccgtggcgctgccgccgttagtaccgatgaaatgggcgatatcattgcccgctatgtagcagaaggggtgtaatcatggctaagacgttatacgaaaaattgttcgacgctcacgttgtgtacgaagccgaaaacgaaaccccactgttatatatcgaccgccacctggtgcatgaagtgacctcaccgcaggcgttcgatggtctgcgcgcccacggtcgcccggtacgtcagccgggcaaaaccttcgctaccatggatcacaacgtctctacccagaccaaagacattaatgcctgcggtgaaatggcgcgtatccagatgcaggaactgatcaaaaactgcaaagaatttggcgtcgaactgtatgacctgaatcacccgtatcaggggatcgtccacgtaatggggccggaacagggcgtcaccttgccggggatgaccattgtctgcggcgactcgcataccgccacccacggcgcgtttggcgcactggcctttggtatcggcacttccgaagttgaacacgtactggcaacgcaaaccctgaaacagggccgcgcaaaaaccatgaaaattgaagtccagggcaaagccgcgccgggcattaccgcaaaagatatcgtgctggcaattatcggtaaaaccggtagcgcaggcggcaccgggcatgtggtggagttttgcggcgaagcaatccgtgatttaagcatggaaggtcgtatgaccctgtgcaatatggcaatcgaaatgggcgcaaaagccggtctggttgcaccggacgaaaccacctttaactatgtcaaaggccgtctgcatgcgccgaaaggcaaagatttcgacgacgccgttgcctactggaaaaccctgcaaaccgacgaaggcgcaactttcgataccgttgtcactctgcaagcagaagaaatttcaccgcaggtcacctggggcaccaatcccggccaggtgatttccgtgaacgacaatattcccgatccggcttcgtttgccgatccggttgaacgcgcgtcggcagaaaaagcgctggcctatatggggctgaaaccgggtattccgctgaccgaagtggctatcgacaaagtgtttatcggttcctgtaccaactcgcgcattgaagatttacgcgcggcagcggagatcgccaaagggcgaaaagtcgcgccaggcgtgcaggcactggtggttcccggctctggcccggtaaaagcccaggcggaagcggaaggtctggataaaatctttattgaagccggttttgaatggcgcttgcctggctgctcaatgtgtctggcgatgaacaacgaccgtctgaatccgggcgaacgttgtgcctccaccagcaaccgtaactttgaaggccgccaggggcgcggcgggcgcacgcatctggtcagcccggcaatggctgccgctgctgctgtgaccggacatttcgccgacattcgcaacattaaataaggagcacaccatggcagagaaatttatcaaacacacaggcctggtggttccgctggatgccgccaatgtcgataccgatgcaatcatcccgaaacagtttttgcagaaagtgacccgtacgggttttggcgcgcatctgtttaacgactggcgttttctggatgaaaaaggccaacagccaaacccggacttcgtgctgaacttcccgcagtatcagggcgcttccattttgctggcacgagaaaacttcggctgtggctcttcgcgtgagcacgcgccctgggcattgaccgactacggttttaaagtggtgattgcgccgagttttgctgacatcttctacggcaatagctttaacaaccagctgctgccggtgaaattaagcgatgcagaagtggacgaactgtttgcgctggtgaaagctaatccggggatccatttcgacgtggatctggaagcgcaagaggtgaaagcgggagagaaaacctatcgctttaccatcgatgccttccgccgccactgcatgatgaacggtctggacagtattgggcttaccttgcagcacgacgacgccattgccgcttatgaagcaaaacaacctgcgtttatgaattaagcggccgcacata

SEQ ID NO: 84 - protein, 363 aa, Escherichia coli
MSKNYHIAVLPGDGIGPEVMTQALKVLDAVRNRFAMRITTSHYDVGGAAIDNHGQPLPPATVEGCEQADAVLFGSVGGPKWEHLPPDQQPERGALLPLRKHFKLFSNLRPAKLYQGLEAFCPLRADIAANGFDILCVRELTGGIYFGQPKGREGSGQYEKAFDTEVYHRFEIERIARIAFESARKRRHKVTSIDKANVLQSSILWREIVNEIATEYPDVELAHMYIDNATMQLIKDPSQFDVLLCSNLFGDILSDECAMITGSMGMLPSASLNEQGFGLYEPAGGSAPDIAGKNIANPIAQILSLALLLRYSLDADDAACAIERAINRALEEGIRTGDLARGAAAVSTDEMGDIIARYVAEGV

SEQ ID NO: 85 - protein, 466 aa, Eshcerichia coli
MAKTLYEKLFDAHVVYEAENETPLLYIDRHLVHEVTSPQAFDGLRAHGRPVRQPGKTFATMDHNVSTQTKDINACGEMARIQMQELIKNCKEFGVELYDLNHPYQGIVHVMGPEQGVTLPGMTIVCGDSHTATHGAFGALAFGIGTSEVEHVLATQTLKQGRAKTMKIEVQGKAAPGITAKDIVLAIIGKTGSAGGTGHVVEFCGEAIRDLSMEGRMTLCNMAIEMGAKAGLVAPDETTFNYVKGRLHAPKGKDFDDAVAYWKTLQTDEGATFDTVVTLQAEEISPQVTWGTNPGQVISVNDNIPDPASFADPVERASAEKALAYMGLKPGIPLTEVAIDKVFIGSCTNSRIEDLRAAAEIAKGRKVAPGVQALVVPGSGPVKAQAEAEGLDKIFIEAGFEWRLPGCSMCLAMNNDRLNPGERCASTSNRNFEGRQGRGGRTHLVSPAMAAAAAVTGHFADIRNIK

SEQ ID NO: 86 - protein, 201 aa, Eshcerichia coli
MAEKFIKHTGLVVPLDAANVDTDAIIPKQFLQKVTRTGFGAHLFNDWRFLDEKGQQPNPDFVLNFPQYQGASILLARENFGCGSSREHAPWALTDYGFKVVIAPSFADIFYGNSFNNQLLPVKLSDAEVDELFALVKANPGIHFDVDLEAQEVKAGEKTYRFTIDAFRRHCMMNGLDSIGLTLQHDDAIAAYEAKQPAFMN

SEQ ID NO: 87 - DNA, 63 bp, Artificial sequence
  PCR primers used to amplify the E. coli threonine dehydratase gene from the pTrcHisA Ec tdcB vector
tcgaattcgcggccgcttctagaaggagatatacatatggctcatattacatacgatctgccg

SEQ ID NO: 88 - DNA, 45 bp, Artificial
  PCR primers used to aomplify the E. coli thronine dehydratase gene from the pTrchsA ec tdcB vector
acgtgcagcggccgctactagtattaggcgtcaacgaaaccggtg

SEQ ID NO: 89 - protein, 507 aa, Pseudomonas sp. KIE171
MSGDYMVQATTNLVGAAYWRGRADEIQFEGRCLIDGKMVEAQSGYTFDCVSPVDGRVLTKVSAGGEADVNRAVAAARSAFRDRRWSGQSPVSRKRTLQAFAALIRLHRDELALLETLDMGKPISASRSVDVEAVASCFEWYAEAIDKLYEQIAPTAETDLALITREPLGVVGAIVPWNFPMLTAAWKTAPALACGNSVVLKPAEQSPLTAIRLAQLALEAGVPEGVFNVVPGLGGSAGRALACHMDVDGIFFTGSTATGRLLTEYAAKSNLKRVCLELGGKSPNIILASYGDIEKAAVASAESMFNNQGEVCIAPSRLIVERSIHKRVVEIVAEVAKRRQPGDPLDPTTRMGALVDANHADRVMGFIGRAKADGATLVAGGTRALTETGGSYVVPTVFDNVSNCMEIARDEVFGPVLSVIPVANVGEAVAVANDSPYGLGAGVWTDRLSDAHKISRELRAGVVYVNCYNDCDITTPFGGVKQSGNGRDKSLYALDEYTELKTTWIKL

SEQ ID NO: 90 - DNA, 1524 bp, Pseudomonas sp. KIE171
atgtccggtgattatatggtacaagcaacaacaaatttggttggcgccgcctattggcgcggtcgtgctgatgaaatccagttcgaaggtcggtgcttaattgatgggaaaatggttgaagcgcagtctgggtatactttcgattgcgtgtcgccggtagacgggcgcgtactgacaaaggtatctgcaggtggcgaggcggacgtcaatagagccgttgctgcagcgcgctcagcatttcgagatcggcgttggtcagggcaatctccagtatcccgcaagcgtactctccaggcattcgcagcgttgattcgcctgcatcgagacgaactggcgctcttggaaacgctcgatatgggtaagcccatttctgcatcacggagcgttgatgtcgaggcagtggcgagttgttttgaatggtatgcagaagcgatcgataagctctacgaacagattgcaccaaccgctgagactgatttagcacttataacccgtgagccattaggggtagtgggcgctattgtgccatggaactttcctatgctgactgcggcttggaagacggcgccggcgctggcttgtgggaattctgtggtgctcaagcctgccgagcaatcgcctctgacagcaattcgtctagcccaattggcgctggaggcgggcgttcccgaaggggtattcaatgtcgtgcctgggcttggtggatctgccggtagggcacttgcctgccacatggatgttgatggtattttctttactggatccaccgcgacaggacgacttctcaccgagtacgctgcgaagagtaacctcaagcgagtttgtctggagctcggagggaagagtccaaatataattctcgcctcctacggtgacattgaaaaagcggctgtagcctcagccgagagtatgttcaataaccaaggggaggtatgcattgctccatctcgattgatagttgaacgctcaatccataaacgggttgtagagattgttgctgaagtcgcaaagcggcgtcagcctggggacccgctcgacccgaccacgcgtatgggcgctttggtggatgctaatcatgccgaccgtgtgatgggatttatcgggcgagcaaaagcggacggcgcaacacttgtggcgggcggtacgcgcgcgctgactgaaaccgggggctcttatgttgtgccaacggtctttgacaacgtgtcaaattgtatggagattgctcgcgacgaagttttcggtccggtgctgtcagtaatccctgtcgccaacgttggcgaggcagttgctgtcgccaacgacagcccttatgggctcggagcaggtgtgtggacggaccgtttatctgatgcgcataagatttcccgagagctgcgggcgggtgttgtgtatgtcaactgttacaacgactgtgacatcaccacaccttttgggggagtcaagcaatccggtaatgggagggacaagtccctttatgcgcttgatgaatatactgaactgaagactacttggatcaagttgtag

SEQ ID NO: 91 - protein, 545 aa, Pseudomonas sp. KIE171
MDPGHAAQRLGVVIGGASSLWEGAYGVGTGGTASPMSGTTKIAREGNTPGSVSSWVAAQNPRKLILVIKTIRKETNVTTFNVISPIDGRELLVGNTSSDAEVAAALNAAETAFKTWKLSSKLERAQLVEALADELLKRADDLSRAVSLSIGRPAAQANETQRFKAVTLAQIEALEELGDERYPSDAQVARFVRRSGQGVHLSIAPWNYPVGLLPWLIVTPILGGNTVILKHAAQTTLIGRIVKEAYEAIGGPAGVLQVLELGHDQVTSAIKSGFVKGVNFIGSVGGGLAVHAAAAGTLTVHAAAAGTLTHVHLELGGKDPAYVRPDADIETAAAEIADGCFSNAGQSCCSVERIYLHEAIRVPFLECFRNEMLKYKLGHPMDPATTVGPVVKASAAEFIRNQIRGAIAMGAEAYVEPALEFSVENASCYLAPTLLTRVAANMHIMQEETFGPVACVQTVRDDAEAISLMNDSKFGLTASVWTRDLDAGLGLVDQLDAGTVFVNRCDHADLYLPWGGQKLSGLGRGNGKEGLLGVMDVKSFHLRAL

SEQ ID NO: 92 - DNA, 1638 bp, Pseudomonas sp. KIE171
atggatcctggtcacgcagcccagcgcttaggggttgtcattggcggcgcttcgtcgctatgggaaggcgcgtacggtgtgggcaccggaggaaccgcgtcgccgatgagtggcacgaccaagattgccagggaaggcaatacccccggctcggtgtcttcttgggttgcagcacaaaatccaagaaagttaattctcgttataaaaacaatccgcaaggagacaaacgtgaccacctttaatgtcatttccccaatcgatggtcgagaactgctggtgggaaacacctccagcgatgccgaagtggccgcagctctcaatgctgcagagactgcattcaagacttggaagctgtcgtccaaattagaacgcgctcagttggttgaagctcttgcggatgagctgcttaagcgcgctgatgatttgtctcgcgcagtttcgctgtcgattggacggcctgccgcgcaggcaaatgaaactcagcgattcaaggctgtaaccttggcacagatcgaggcgctcgaagagcttggtgatgagcgatatccttccgatgcgcaggtggcgcgttttgtgcgtcgtagtggtcaaggggtacatctttcgatcgctccctggaactaccctgtcggtctgcttccctggctgatcgttacaccaattcttggtggtaacacagtgatcttgaagcacgcggctcagacgacccttattgggaggattgtcaaagaggcgtatgaagcaatcggagggcctgctggcgtgctccaagtgcttgagcttggtcacgaccaggttacgagtgctatcaaatccggctttgtcaaaggggtgaacttcatcggatctgtcggaggcggcctcgccgttcacgcagcggcggctggaactttgaccgttcacgcagcggcggctggaactttgacccatgtccatctggagcttgggggtaaggacccggcctatgttcgcccggatgcggatatcgaaacggcagcagccgagattgccgacggttgcttctcaaacgccggtcaatcctgctgttcggtagagcgtatttatctgcacgaagcgattcgggtaccgttcctggagtgcttccgtaacgaaatgctgaagtacaagttggggcacccaatggaccccgcaactacggttggtcccgtcgtcaaggcttcggctgcagaattcattcgcaaccaaatccgaggggccattgccatgggggctgaggcgtatgtcgaacccgcgctggagttctcggtggagaacgcctcctgctatctggccccgactcttttgactcgcgtagcggccaatatgcacatcatgcaagaggaaacgtttggaccggtcgcctgtgttcaaacggtgcgagacgacgcagaggccattagtttgatgaacgacagtaagttcggtctgaccgcaagcgtctggacacgcgatctggacgcagggctcggtttggtcgatcagcttgatgccggcacagtattcgtgaaccgttgtgaccatgcagacctttacctgccttggggtgggcagaagctctctggtctgggtcgcggcaatggtaaagaaggacttcttggggtcatggacgtaaagtcgttccacctgagagctttgtaa

SEQ ID NO: 93 - protein, 524 aa, Propionibacterium acidipropionici
MANKKRVKLAETMEGRLEQLTEQRHKIELGGGEKRLQKQRDKGKQTARERIDNLLDEYSFDEVGAFREHRTSLFGMDTAEVPADGVVTGRGTVNGRPVHVASQDFSVMGGSAGETQSTKVVETMEQALLTGTPFLFFYDSGGARIQEGIDSLSGYGKMFYANVKLSGVVPQIAIIAGPCAGGASYSPALTDFIIMTKKAQMFITGPGVIKSVTGEDVTGDELGGAEAHMSTSGNIHFVAEDDDAAVLIAQKLLSFLPQNNTQDATIEFPNNDISPIPELRDIVPIDGKKGYDVRDVISKIVDWGDYLEVKAGWATNIVTAFARINGRSVGIVANQPKVMSGCLDINASDKAAEFINFCDSFNIPLVQLVDVPGFLPGVQQEYGGIIRHGAKMLYAYSEATVPKITVVLRKAYGGSYLAMCNRDLGADAVYAWPTAEIAVMGAEGAANVIFRREIKASDDPAATRAEKIEEYQTAFNTPYVAAARGQVDDVIDPADTRRRITAALETYATKRQSRPAKKHGNMPC

SEQ ID NO: 94 - DNA, 1600 bp, Propionibacterium acidipropionici
  misc_feature (1578)..(1578): n is a, c, g, or t
atggctaacaagaagcgcgtcaagctcgccgagacgatggaaggtcgtctcgagcagctgaccgaacagcgtcacaagattgagctcggaggtggcgagaagcgcctccagaagcagcgcgacaagggcaagcagaccgcccgcgagcggatcgacaacctcctcgacgagtactccttcgacgaggtcggcgccttccgtgagcaccgcaccagcctcttcgggatggacaccgccgaggtgcccgccgacggcgtggtgaccggccgcggaaccgtcaacggccgcccggtccacgtggcctcccaggacttctccgtcatgggcgggtcggccggcgagacccagtcgaccaaggtcgtcgagaccatggagcaggcgctgctgaccggcacaccgttcctcttcttctacgattcgggcggcgctcggatccaggagggcatcgactcgctgtccggctacggcaagatgttctacgcgaacgtcaagctgtcgggtgtcgtgccgcagatcgccatcatcgccggcccctgcgccggtggtgcgtcctactccccggccctgaccgacttcatcatcatgacgaagaaggcccagatgttcatcacgggccccggcgtcatcaagtcggtgaccggcgaggacgtgaccggtgacgagctgggtggtgccgaggcgcacatgtcgacctccggcaacatccacttcgtggccgaggacgacgacgccgcggtgctgatcgcgcagaagctgctgagcttcctgccgcagaacaacacccaggacgcgacgatcgagttcccgaacaacgacatctccccgatccccgaactgcgcgacatcgtgccgatcgacgggaagaagggatatgacgtccgcgacgtcatctccaagatcgtcgactggggcgactacctggaggtcaaggcgggctgggcgaccaacatcgtcaccgccttcgccaggatcaacggtcgttcggtgggcatcgtggcgaaccagcccaaggtgatgtcgggctgcctcgacatcaacgcctcggacaaggccgcggagttcatcaacttctgcgactcgttcaacatcccgctggtgcagctggtggacgtccccggattccttccgggcgtccagcaggagtacggcggcatcatccgccatggcgcgaagatgctgtacgcctactccgaggccaccgtcccgaagatcaccgtggtgctgcgcaaggcctacggcggctcctacctggccatgtgcaaccgtgacctgggtgccgacgccgtctacgcctggcccaccgccgagatcgccgtgatgggcgccgagggcgcagccaatgtgatcttccgtcgtgagatcaaggcctccgatgatcccgccgccacccgcgcggagaagatcgaggagtaccagacggcgttcaacacgccgtatgtggcggctgcccgcggacaggtcgatgacgtcatcgaccccgccgacacccgtcgcaggatcactgccgctctggagacctacgccacgaagcgtcagtcccgtccggccaagaagcacggcaatatgccgtgctgabtndndnttranscarbyasssbnt

SEQ ID NO: 95 - protein, 497 aa, Propionibacterium acidipropionici
MSPRKIGVTELALRDAHQSLIATRMSMEDMVDACADIDAAGYWSAECWGGATFDSCIRFLNEDPWERLRTFRKLMPNTRLQMLLRGQNLLGYRHYGDDVVDKFVEKSAENGMDVFRVFDALNDPRNLERAMAAVKKTGKHAQGTICYTTSPIHTPESFVKQADRLIDMGADSIAFKDMAALLKPQPAYDIIKGIKENHPDVQINLHCHSTTGVTLVTLMKAIEAGVDVVDTAISSMSLGPGHNPTESLVEMLEGTGYETGLDMDRLIKIRDHFKKVRPKYKKFESKTLVNTNIFQSQIPGGMLSNMESQLAAQGASDRTDEVMKEVPRVRKDAGYPPLVTPSSQIVGTQAVFNVLMGNGSYKNLTAEFADLMLGYYGKPVGELNPELIKMAEKQTGKKPIDVRPADLIDNEWDDLVKQSAELEGFDGSDEDVLTNALFPGVAPKFFKERPQGPKSVAMTEAQMKAEAEGNGSTSVSGPVNYSVTVGGRSHDVTVEPA

SEQ ID NO: 96 - DNA, 1494 bp, Propionibacterium acidipropionici
atgagtccgcgaaagattggcgttaccgagctcgcgctccgcgacgcgcatcagagcctgattgcaacccggatgtccatggaggacatggtcgatgcctgtgccgacatcgatgcggctggctactggtccgctgagtgctggggtggagctaccttcgattcctgtatccggttcctcaacgaggatccctgggagaggctgcggaccttccgcaagctgatgcccaacacccgcctgcagatgctgctgcgcggccagaacctcctcggctaccgccactacggtgacgacgtcgtcgacaagttcgtcgagaagtcggccgagaacggcatggacgtcttccgtgtcttcgacgccctcaacgatccccgcaacctcgagcgcgcgatggccgccgtcaagaagaccggcaagcacgcccagggcacgatctgctacaccacctccccgatccacaccccggagagcttcgtcaagcaggccgaccgcctcatcgacatgggtgccgactccatcgccttcaaggacatggccgccctgctgaagccgcagcccgcctatgacatcatcaagggcatcaaggagaaccaccccgacgtgcagatcaacctgcactgccactccaccaccggcgtcaccctggtgaccctcatgaaggccatcgaggccggcgtcgacgtcgtcgacaccgccatctcctcgatgtcgctggggccgggccacaacccgaccgagtccctggtcgagatgctcgagggcaccggctacgagaccggcctggacatggatcgcctcatcaagatccgcgaccacttcaagaaggtccgcccgaagtacaagaagttcgagtcgaagaccctggtcaacaccaacatcttccagtcccagatccccggcggcatgctctccaacatggagtcccagctggccgcccagggcgcctcggaccgcaccgacgaggtcatgaaggaggtgccgcgcgtccgcaaggatgccggctacccgcccctggtcaccccgtcctcccagatcgtcggcacccaggccgtgttcaacgtcctgatgggcaacggctcctacaagaacctgaccgccgagttcgccgacctgatgctcggctactacggcaagccggtcggcgagctcaaccccgagctcatcaagatggccgagaagcagaccggcaagaagccgatcgacgtgcgcccggccgatctcatcgataacgagtgggacgacctggtcaagcagtccgccgagctcgagggcttcgacggatccgacgaggacgtgctcaccaacgccctgttcccgggagtcgccccgaagttcttcaaggagcgcccgcagggccccaagagcgtcgcgatgaccgaggctcagatgaaggccgaggccgagggcaacggatcgacgtccgtctccggtccggtcaattacagcgtcacggtcggtggccgcagccacgacgtgaccgtcgagcctgcgtga

SEQ ID NO: 97 - protein, 121 aa, Propionibacterium acidipropionici
MKLKVTVNGVAYDVDVDVDKTNNSPIPPIIFGGGSGGPARAAGGSGGGKAGAGEIPAPLAGTVAKILVKEGDQVKAGDVVLTLEAMKMETEITATSDGTVKSILVAVGDAVQGGQGLVAVG

SEQ ID NO: 98 - DNA, 366 bp, Propionibacterium acidipropionici
atgaagctcaaggtgactgtcaacggcgtcgcttatgacgtcgacgttgacgttgacaagaccaacaattccccgatcccgccgatcatcttcggcggcgggtccggcggcccggctcgcgctgcgggcgggtccggcggtggcaaggccggtgcgggcgagattcccgccccgctggccggtaccgtcgccaagatcctcgtgaaggagggggaccaggtgaaggccggcgatgtcgtgctgactctcgaggccatgaagatggagaccgagatcaccgcgacctccgacgggaccgtcaagagcatcctggtcgccgtcggcgacgccgtccagggtggccagggcctggtggccgtcggctga

SEQ ID NO: 99 - protein, 77 aa, Propionibacterium acidipropionici
MTDNEKDRVIETLTKRLTALEADVSRLKAESADVPEDVVTVISAAVAAYLGNDGKVQAIRFAPSENWTRQGRRALQN

SEQ ID NO: 100 - DNA, 246 bp, Propionibacterium acidipropionici
atgactgacaacgagaaggatcgcgtcatcgagacgctgaccaagcggctcaccgcgctggaggccgacgtcagccggctcaaggccgagagcgccgacgtccccgaggacgtcgtgaccgtgatcagcgccgccgtggcggcctacctgggcaatgacgggaaggttcaggccatccgtttcgccccgagtgagaactggacccgtcagggtcgcagagcgctccagaaccattccattcgttga

SEQ ID NO: 101 - protein, 524 aa, Propionibacterium freudenreichii
MAENNNLKLASTMEGRVEQLAEQRQVIEAGGGERRVEKQHSQGKQTARERLNNLLDPHSFDEVGAFRKHRTTLFGMDKAVVPADGVVTGRGTILGRPVHAASQDFTVMGGSAGETQSTKVVETMEQALLTGTPFLFFYDSGGARIQEGIDSLSGYGKMFFANVKLSGVVPQIAIIAGPCAGGASYSPALTDFIIMTKKAHMFITGPQVIKSVTGEDVTADELGGAEAHMAISGNIHFVAEDDDAAELIAKKLLSFLPQNNTEEASFVNPNNDVSPNTELRDIVPIDGKKGYDVRDVIAKIVDWGDYLEVKAGYATNLVTAFARVNGRSVGIVANQPSVMSGCLDINASDKAAEFVNFCDSFNIPLVQLVDVPGFLPGVQQEYGGIIRHGAKMLYAYSEATVPKITVVLRKAYGGSYLAMCNRDLGADAVYAWPSAEIAVMGAEGAANVIFRKEIKAADDPDAMRAEKIEEYQNAFNTPYVAAARGQVDDVIDPADTRRKIASALEMYATKRQTRPAKKHGNFPC

SEQ ID NO: 102 - DNA, 1575 bp, Propionibacterium freudenreichii
atggctgaaaacaacaatttgaagctcgccagcaccatggaaggtcgcgtggagcagctcgcagagcagcgccaggtgatcgaagccggtggcggcgaacgtcgcgtcgagaagcaacattcccagggtaagcagaccgctcgtgagcgcctgaacaacctgctcgatccccattcgttcgacgaggtcggcgctttccgcaagcaccgcaccacgttgttcggcatggacaaggccgtcgtcccggcagatggcgtggtcaccggccgtggcaccatccttggtcgtcccgtgcacgccgcgtcccaggacttcacggtcatgggtggttcggctggcgagacgcagtccacgaaggtcgtcgagacgatggaacaggcgctgctcaccggcacgcccttcctgttcttctacgattcgggcggcgcccggatccaggagggcatcgactcgctgagcggttacggcaagatgttcttcgccaacgtgaagctgtcgggcgtcgtgccgcagatcgccatcattgccggcccctgtgccggtggcgcctcgtattcgccggcactgactgacttcatcatcatgaccaagaaggcccatatgttcatcacgggcccccaggtcatcaagtcggtcaccggcgaggatgtcaccgctgacgaactcggtggcgctgaggcccatatggccatctcgggcaatatccacttcgtggccgaggacgacgacgccgcggagctcattgccaagaagctgctgagcttccttccgcagaacaacactgaggaagcatccttcgtcaacccgaacaatgacgtcagccccaataccgagctgcgcgacatcgttccgattgacggcaagaagggctatgacgtgcgcgatgtcattgccaagatcgtcgactggggtgactacctcgaggtcaaggccggctatgccaccaacctcgtgaccgccttcgcccgggtcaatggtcgttcggtgggcatcgtggccaatcagccgtcggtgatgtcgggttgcctcgacatcaacgcctctgacaaggccgccgaattcgtgaatttctgcgattcgttcaacatcccgctggtgcagctggtcgacgtgccgggcttcctgcccggcgtgcagcaggagtacggcggcatcattcgccatggcgcgaagatgctgtacgcctactccgaggccaccgtgccgaagatcaccgtggtgctccgcaaggcctacggcggctcctacctggccatgtgcaaccgtgaccttggtgccgacgccgtgtacgcctggcccagcgccgagattgcggtgatgggcgccgagggtgcggcaaatgtgatcttccgcaaggagatcaaggctgccgacgatcccgacgccatgcgcgccgagaagatcgaggagtaccagaacgcgttcaacacgccgtacgtggccgccgcccgcggtcaggtcgacgacgtgattgacccggctgatacccgtcgaaagattgcttccgccctggagatgtacgccaccaagcgtcagacccgcccggcgaagaagcatggaaacttcccctgctga

SEQ ID NO: 103 - protein, 505 aa, Propionibacterium freudenreichii
MSPREIEVSEPREVGITELVLRDAHQSLMATRMAMEDMVGACADIDAAGYWSVECWGGATYDSCIRFLNEDPWERLRTFRKLMPNSRLQMLLRGQNLLGYRHYNDEVVDRFVDKSAENGMDVFRVFDAMNDPRNMAHAMAAVKKAGKHAQGTICYTISPVHTVEGYVKLAGQLLDMGADSIALKDMAALLKPQPAYDIIKAIKDTYGQKTQINLHCHSTTGVTEVSLMKAIEAGVDVVDTAISSMSLGPGHNPTESVAEMLEGTGYTTNLDYDRLHKIRDHFKAIRPKYKKFESKTLVDTSIFKSQIPGGMLSNMESQLRAQGAEDKMDEVMAEVPRVRKAAGFPPLVTPSSQIVGTQAVFNVMMGEYKRMTGEFADIMLGYYGASPADRDPKVVKLAEEQSGKKPITQRPADLLPPEWEEQSKEAAALKGFNGTDEDVLTYALFPQVAPVFFEHRAEGPHSVALTDAQLKAEAEGDEKSLAVAGPVTYNVNVGGTVREVTVQQA

SEQ ID NO: 104 - DNA, 1518 bp, Propionibacterium freudenreichii
atgagtccgcgagaaattgaggtttccgagccgcgcgaggttggtatcaccgagctcgtgctgcgcgatgcccatcagagcctgatggccacacgaatggcaatggaagacatggtcggcgcctgtgcagacattgatgctgccgggtactggtcagtggagtgttggggtggtgccacgtatgactcgtgtatccgcttcctcaacgaggatccttgggagcgtctgcgcacgttccgcaagctgatgcccaacagccgtctccagatgctgctgcgtggccagaacctgctgggttaccgccactacaacgacgaggtcgtcgatcgcttcgtcgacaagtccgctgagaacggcatggacgtgttccgtgtcttcgacgccatgaatgatccccgcaacatggcgcacgccatggctgccgtcaagaaggccggcaagcacgcgcagggcaccatttgctacacgatcagcccggtccacaccgttgagggctatgtcaagcttgctggtcagctgctcgacatgggtgctgattccatcgccctgaaggacatggccgccctgctcaagccgcagccggcctacgacatcatcaaggccatcaaggacacctacggccagaagacgcagatcaacctgcactgccactccaccacgggtgtcaccgaggtctccctcatgaaggccatcgaggccggcgtcgacgtcgtcgacaccgccatctcgtccatgtcgctcggcccgggccacaaccccaccgagtcggttgccgagatgctcgagggcaccgggtacaccaccaaccttgactacgatcgcctgcacaagatccgcgatcacttcaaggccatccgcccgaagtacaagaagttcgagtcgaagacgcttgtcgacacctcgatcttcaagtcgcagatccccggcggcatgctctccaacatggagtcgcagctgcgcgcccagggcgccgaggacaagatggacgaggtcatggcagaggtgccgcgcgtccgcaaggccgccggcttcccgcccctggtcaccccgtccagccagatcgtcggcacgcaggccgtgttcaacgtgatgatgggcgagtacaagaggatgaccggcgagttcgccgacatcatgctcggctactacggcgccagcccggccgatcgcgatccgaaggtggtcaagttggccgaggagcagtccggcaagaagccgatcacccagcgcccggccgatctgctgccccccgagtgggaggagcagtccaaggaggccgcggccctcaagggcttcaacggcaccgacgaggacgtgctcacctatgcactgttcccgcaggtcgctccggtcttcttcgagcatcgcgccgagggcccgcacagcgtggctctcaccgatgcccagctgaaggccgaggccgagggcgacgagaagtcgctcgccgtggccggtcccgtcacctacaacgtgaacgtgggcggaaccgtccgcgaagtcaccgttcagcaggcgtga

SEQ ID NO: 105 - protein, 123 aa, Propionibacterium freudenreichii
MKLKVTVNGTAYDVDVDVDKSHENPMGTILFGGGTGGAPAPRAAGGAGAGKAGEGEIPAPLAGTVSKILVKEGDTVKAGQTVLVLEAMKMETEINAPTDGKVEKVLVKERDAVQGGQGLIKIG

SEQ ID NO: 106 - DNA, 372 bp, Propionibacterium freudenreichii
atgaaactgaaggtaacagtcaacggcactgcgtatgacgttgacgttgacgtcgacaagtcacacgaaaacccgatgggcaccatcctgttcggcggcggcaccggcggcgcgccggcaccgcgcgcagcaggtggcgcaggcgccggtaaggccggagagggcgagattcccgctccgctggccggcaccgtctccaagatcctcgtgaaggagggtgacacggtcaaggctggtcagaccgtgctcgttctcgaggccatgaagatggagaccgagatcaacgctcccaccgacggcaaggtcgagaaggtccttgtcaaggagcgtgacgccgtgcagggcggtcagggtctcatcaagatcggctga

SEQ ID NO: 107 - protein, 1167 aa, Ralstonia eutropha; identical to SEQ ID NO: 59

SEQ ID NO: 108 - DNA, 3504 bp, Ralstonia eutropha; identical to SEQ ID NO: 60

SEQ ID NO: 109 - protein, 327 aa, Propionibacterium acidipropionici
MSTAPVKIAVTGAAGQICYSLLFRIASGSLLGSTPIELRLLEITPALKALEGVVMELDDGAFPNLVNIEIGDDPKKVFDGVNAAFLVGAMPRKAGMERSDLLSKNGAIFTAQGKALNDVAADDVKVLVTGNPANTNALIAATNAVDIPNDHFAALTRLDHNRAKTQLARKVGAGVADVKHMTIWGNHSSTQYPDVFHAEVAGKSAADLVDEAWVENEFIPTVAKRGAAIIAARGSSSAASAANATVECMHDWLGSTPEGDWVSMAVPSDGSYGVPEGLISSFPVTVSDGKVEIVQGLDIDSFSRGKIDASAAELQDERDAVKELGLI

SEQ ID NO: 110 - DNA, 984 bp, Propionibacterium acidipropionici
atgagcactgctcccgtcaagattgctgtgaccggcgccgccggtcagatctgttacagcctgttgttccgcatcgccagtggttcgctgctcggcagcacccccatcgagctgcgtctgctggagatcacccccgctctcaaggctctcgagggtgtcgtcatggagctcgatgacggtgccttcccgaacctcgtcaacatcgagatcggcgatgaccccaagaaggtcttcgacggcgtcaacgccgccttcctggtcggcgccatgccccgcaaggccggcatggagcgctccgatctgctgagcaagaacggcgcgatcttcaccgctcagggcaaggccctcaatgacgtcgccgccgacgacgtcaaggtcctggtgaccggcaacccggccaacaccaacgccctgatcgcggccaccaacgccgtggacatcccgaacgaccacttcgccgccctgacccgtctggaccacaaccgcgccaagacccagctggcccgcaaggtcggcgccggcgtggccgacgtcaagcacatgaccatctggggcaaccactcctccacccagtaccccgacgtcttccacgccgaggtcgcgggcaagagcgctgccgatctggtcgacgaggcctgggtcgagaacgagttcatcccgactgtcgccaagcgcggcgccgctatcatcgccgcccgcggttcctcttctgccgcctcggccgccaacgcgaccgtcgagtgcatgcacgactggcttggcagcacccccgagggcgactgggtctcgatggcagttccgtccgacggctcctacggggtgcccgagggcctcatctcgtccttcccggtcaccgtctccgacggcaaggtcgagatcgtccagggcctggacatcgactccttctcccgcggcaagatcgacgcctccgcagctgagctgcaggatgagcgcgacgccgtcaaggagctcggcctcatctga

SEQ ID NO: 111 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVAKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 112 - DNA, 939 bp, Escherichia coli
atgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcgcgtaaaccgggtatggatcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaa

SEQ ID NO: 113 - protein, 548 aa, Escherichia coli
MSNKPFHYQAPFPLKKDDTEYYLLTSEHVSVSEFEGQEILKVAPEALTLLARQAFHDASFMLRPAHQQQVADILRDPEASENDKYVALQFLRNSDIAAKGVLPTCQDTGTAIIVGKKGQRVWTGGGDEAALARGVYNTYIEDNLRYSQNAPLDMYKEVNTGTNLPAQIDLYAVDGDEYKFLCIAKGGGSANKTYLYQETKALLTPGKLKNYLVEKMRTLGTAACPPYHIAFVIGGTSAETNLKTVKLASAKYYDELPTEGNEHGQAFRDVELEKELLIEAQNLGLGAQFGGKYFAHDIRVIRLPRHGASCPVGMGVSCSADRNIKAKINRQGIWIEKLEHNPGKYIPEELRKAGEGEAVRVDLNRPMKEILAQLSQYPVSTRLSLNGTIIVGRDIAHAKLKERMDNGEGLPQYIKDHPIYYAGPAKTPEGYASGSLGPTTAGRMDSYVDQLQAQGGSMIMLAKGNRSQQVTDACKKHGGFYLGSIGGPAAVLAQGSIKSLECVEYPELGMEAIWKIEVEDFPAFILVDDKGNDFFQQIQLTQCTRCVK

SEQ ID NO: 114 - DNA, 1647 bp, Escherichia coli
atgtcaaacaaaccctttcattatcaggctccttttccactcaaaaaagatgatactgagtattacctgctaaccagcgaacacgttagcgtatctgaatttgaagggcaggagattttgaaagtcgcacccgaagcgttaactctgttggcgcgccaggcgtttcatgatgcgtcgttcatgctgcgtccggcgcaccaacaacaggtggccgacattctgcgtgacccggaggccagcgaaaatgataaatatgtggcgctgcaattcctgcgtaactccgacatcgcggcgaaaggcgttctgccaacctgtcaggataccggcaccgcgattattgttggtaaaaaagggcagcgtgtatggaccggtggtggtgatgaagcggcgctggcgcgcggtgtctataacacttatatcgaagataatctgcgctactcgcaaaacgcgccgctggatatgtataaagaagtgaataccggcaccaatctgccagcgcagatcgatctttatgccgttgatggcgacgaatacaaattcctctgtatcgccaaaggtggtggttcggcaaacaagacgtatctctatcaggaaaccaaagcgttactgacgccggggaaactgaaaaattacctggttgagaagatgcgcacgctgggtacggcggcctgtcctccgtatcatattgcgttcgttattggtggaacttctgcagaaacgaaccttaaaacggtgaaactggcttccgcgaaatactatgatgaactgccaacggaagggaatgagcacggtcaggcgttccgcgatgtggaactggaaaaagaattgctgatcgaagcgcaaaatcttggtctgggtgcgcagtttggtggtaaatacttcgctcacgacatccgcgtgattcgcctgccacgtcacggcgcatcctgcccggtcggtatgggcgtctcctgctctgctgaccgtaatatcaaagcgaagatcaaccgtcaggggatctggatcgaaaaactggaacataatccaggcaaatatatcccggaagagctgcgcaaagcgggagaaggcgaagcggtgcgcgttgaccttaaccgtccgatgaaagagatcctcgcacagttgtcgcagtatcccgtttctacacgcttatcgcttaacggcacgattatcgtcggtcgtgatattgctcacgccaaactgaaagagcggatggataacggtgaagggctgccgcagtacatcaaagatcatccgatttactacgcgggtccggccaaaacgccggaaggttatgcctccggttctcttggcccaacgaccgccggacggatggattcttatgtcgatcaactgcaagcgcagggcggaagtatgatcatgctggcgaaaggcaaccgcagccagcaggtgacggatgcctgtaaaaaacacggcggcttctaccttggcagtatcggtggtccggccgctgtattggcgcagggaagtattaagagcctggaatgtgttgaatatccggaactgggaatggaagccatctggaaaattgaagtggaagatttcccggcgtttatccttgtggatgataaaggaaatgacttcttccagcagatacaactcacacaatgcacccgctgtgtgaaataa

SEQ ID NO: 115 - protein, 548 aa, Escherichia coli
MSNKPFIYQAPFPMGKDNTEYYLLTSDYVSVADFDGETILKVEPEALTLLAQQAFHDASFMLRPAHQKQVAAILHDPEASENDKYVALQFLRNSEIAAKGVLPTCQDTGTAIIVGKKGQRVWTGGGDEETLSKGVYNTYIEDNLRYSQNAALDMYKEVNTGTNLPAQIDLYAVDGDEYKFLCVAKGGGSANKTYLYQETKALLTPGKLKNFLVEKMRTLGTAACPPYHIAFVIGGTSAETNLKTVKLASAHYYDELPTEGNEHGQAFRDVQLEQELLEEAQKLGLGAQFGGKYFAHDIRVIRLPRHGASCPVGMGVSCSADRNIKAKINREGIWIEKLEHNPGQYIPQELRQAGEGEAVKVDLNRPMKEILAQLSQYPVSTRLSLTGTIIVGRDIAHAKLKELIDAGKELPQYIKDHPIYYAGPAKTPAGYPSGSLGPTTAGRMDSYVDLLQSHGGSMIMLAKGNRSQQVTDACHKHGGFYLGSIGGPAAVLAQQSIKHLECVAYPELGMEAIWKIEVEDFPAFILVDDKGNDFFQQIVNKQCANCTK

SEQ ID NO: 116 - DNA, 1647 bp, Escherichia coli
atgtcaaacaaaccctttatctaccaggcacctttcccgatggggaaagacaataccgaatactatctactcacttccgattacgttagcgttgccgacttcgacggcgaaaccatcctgaaagtggaaccagaagccctgaccctgctggcgcagcaagcctttcacgacgcttcttttatgctccgcccggcacaccagaaacaggttgcggctattcttcacgatccagaagccagcgaaaacgacaagtacgtggcgctgcaattcttaagaaactccgaaatcgccgccaaaggcgtgctgccgacctgccaggataccggcaccgcgatcatcgtcggtaaaaaaggccagcgcgtgtggaccggcggcggtgatgaagaaacgctgtcgaaaggcgtctataacacctatatcgaagataacctgcgctattcacagaatgcggcgctggacatgtacaaagaggtcaacaccggcactaacctgcctgcgcaaatcgacctgtacgcggtagatggcgatgagtacaaattcctttgcgttgcgaaaggcggcggctctgccaacaaaacgtatctctaccaggaaaccaaagccctgctgactcccggcaaactgaaaaacttcctcgtcgagaaaatgcgtaccctcggtactgcagcctgcccgccgtaccatatcgcgtttgtgattggcggtacgtctgcggaaaccaacctgaaaaccgtcaagttagcaagcgctcactattacgatgaactgccgacggaagggaacgaacatggtcaggcgttccgcgatgtccagctggaacaggaactgctggaagaggcccagaaactcggtcttggcgcgcagtttggcggtaaatacttcgcgcacgacattcgcgttatccgtctgccacgtcacggcgcatcctgcccggtcggcatgggcgtctcctgctccgctgaccgtaacattaaagcgaaaatcaaccgcgaaggtatctggatcgaaaaactggaacacaacccaggccagtacattccacaagaactgcgccaggccggtgaaggcgaagcggtgaaagttgaccttaaccgcccgatgaaagagatcctcgcccagctttcgcaatacccggtatccactcgtttgtcgctcaccggcaccattatcgtgggccgagatattgcacacgccaagctgaaagagctgattgacgccggtaaagaacttccgcagtacatcaaagatcacccgatctactacgcgggtccggcgaaaacccctgccggttatccatcaggttcacttggcccaaccaccgcaggccgtatggactcctacgtggatctgctgcaatcccacggcggcagcatgatcatgctggcgaaaggtaaccgcagtcagcaggttaccgacgcgtgtcataaacacggcggcttctacctcggtagcatcggcggtccggcggcggtactggcgcagcagagcatcaagcatctggagtgcgtcgcttatccggagctgggtatggaagctatctggaaaatcgaagtagaagatttcccggcgtttatcctggtcgatgacaaaggtaacgacttcttccagcaaatcgtcaacaaacagtgcgcgaactgcactaagtaa

SEQ ID NO: 117 - protein, 467 aa, Escherichia coli
MNTVRSEKDSMGAIDVPADKLWGAQTQRSLEHFRISTEKMPTSLIHALALTKRAAAKVNEDLGLLSEEKASAIRQAADEVLAGQHDDEFPLAIWQTGSGTQSNMNMNEVLANRASELLGGVRGMERKVHPNDDVNKSQSSNDVFPTAMHVAALLALRKQLIPQLKTLTQTLNEKSRAFADIVKIGRTHLQDATPLTLGQEISGWVAMLEHNLKHIEYSLPHVAELALGGTAVGTGLNTHPEYARRVADELAVITCAPFVTAPNKFEALATCDALVQAHGALKGLAASLMKIANDVRWLASGPRCGIGEISIPENEPGSSIMPGKVNPTQCEALTMLCCQVMGNDVAINMGGASGNFELNVFRPMVIHNFLQSVRLLADGMESFNKHCAVGIEPNRERINQLLNESLMLVTALNTHIGYDKAAEIAKKAHKEGLTLKAAALALGYLSEAEFDSWVRPEQMVGSMKAGR

SEQ ID NO: 118 - DNA, 1404 bp, Escherichia coli
atgaatacagtacgcagcgaaaaagattcgatgggggcgattgatgtcccggcagataagctgtggggcgcacaaactcaacgctcgctggagcatttccgcatttcgacggagaaaatgcccacctcactgattcatgcgctggcgctaaccaagcgtgcagcggcaaaagttaatgaagatttaggcttgttgtctgaagagaaagcgagcgccattcgtcaggcggcggatgaagtactggcaggacagcatgacgacgaattcccgctggctatctggcagaccggctccggcacgcaaagtaacatgaacatgaacgaagtgctggctaaccgggccagtgaattactcggcggtgtgcgcgggatggaacgtaaagttcaccctaacgacgacgtgaacaaaagccaaagttccaacgatgtctttccgacggcgatgcacgttgcggcgctgctggcgctgcgcaagcaactcattcctcagcttaaaaccctgacacagacactgaatgagaaatcccgtgcttttgccgatatcgtcaaaattggtcgtactcacttgcaggatgccacgccgttaacgctggggcaggagatttccggctgggtagcgatgctcgagcataatctcaaacatatcgaatacagcctgcctcacgtagcggaactggctcttggcggtacagcggtgggtactggactaaatacccatccggagtatgcgcgtcgcgtagcagatgaactggcagtcattacctgtgcaccgtttgttaccgcgccgaacaaatttgaagcgctggcgacctgtgatgccctggttcaggcgcacggcgcgttgaaagggttggctgcgtcactgatgaaaatcgccaatgatgtccgctggctggcctctggcccgcgctgcggaattggtgaaatctcaatcccggaaaatgagccgggcagctcaatcatgccggggaaagtgaacccaacacagtgtgaggcattaaccatgctctgctgtcaggtgatggggaacgacgtggcgatcaacatggggggcgcttccggtaactttgaactgaacgtcttccgtccaatggtgatccacaatttcctgcaatcggtgcgcttgctggcagatggcatggaaagttttaacaaacactgcgcagtgggtattgaaccgaatcgtgagcgaatcaatcaattactcaatgaatcgctgatgctggtgactgcgcttaacacccacattggttatgacaaagccgccgagatcgccaaaaaagcgcataaagaagggctgaccttaaaagctgcggcccttgcgctggggtatcttagcgaagccgagtttgacagctgggtacggccagaacagatggtcggcagtatgaaagccgggcgttaa

SEQ ID NO: 119 - protein, 474 aa, Propionibacterium freudenreichii
MADNTSAKTRTESDSMGTVEVPANHHWGAQTERSLHNFDIGRPTFVWGRPMIKALGILKKAAAQANGELGELPKDISELIVKAADDVIAGKLDDDFPLVVFQTGSGTQSNMNANEVISNRAIEIAGGEMGTKTPVHPNDHVNRGQSSNDTFPTAMHIAVVTELQEMYPRVMKLRDTLDAKAKEYDDVVMVGRTHLQDATPIRLGQVISGWVAQIDFALKCIKFSDEQARELAIGGTAVGTGLNAHPKFGPLTAEKISDETGLKFEQAPNLFAALSAHDALVQVSGSLRVLGDALMKIANDVRWYASGPRNGIGELLIPENEPGSSIMPGKVNPTQCEAMTMVATKVFGNDATVGFAGSQGNFQLNVFKPVMAWCVLESIQLLGDTCVSFNDHCAVGIEPNLEKIKHNLDINLMQVTALNRHIGYDKASKIAKNAHHKGIGLRDSALELGFLTPEEFDKWVVPADMTHPSAADDD

SEQ ID NO: 120 - DNA, 1425 bp, Propionibacterium freudenreichii
atggctgataacaccagcgcgaagacgcgcacggaatccgactccatgggcaccgtcgaggtgccggcaaaccaccattggggggcgcagaccgagcgcagtctgcacaacttcgacatcggtcgtccgaccttcgtgtggggacgcccgatgatcaaggccctcggcatcctgaagaaggctgccgcccaggccaatggagagctcggggagctccccaaggacatctccgagctcatcgtcaaggccgccgatgacgtgatcgccggcaagctcgacgacgacttccccctggtggtcttccagaccggctcgggcacgcagtcgaacatgaacgccaatgaggtgatctccaaccgcgcgatcgagatcgccggcggcgagatgggcaccaagaccccggtgcaccccaatgaccacgtgaaccgtggccagtccagcaacgacaccttccccacggcgatgcacattgccgtggtcaccgagctgcaggagatgtacccgcgcgtgatgaagctgcgcgacaccctggacgccaaggccaaggaatatgacgatgtcgtgatggtggggcgcacccacctgcaggacgcgaccccgatccgcctcggccaggtgatcagcggctgggtggcccagatcgacttcgccctcaagtgcatcaagttctccgacgagcaggcacgcgaactcgccatcggcggcaccgccgtcggcaccggcctgaacgcgcatccgaagttcggcccgctcaccgccgagaagatcagcgacgagaccggcctcaagttcgagcaggccccgaacctgttcgccgcactgagcgcccacgacgcgctggtgcaggtctccggttcgctgcgcgtgctgggcgacgccctgatgaagatcgccaacgacgtgcgttggtatgcctccggcccccgcaatggcatcggcgagctgctgatccccgagaacgagcccggcagctcgatcatgcccggcaaggtcaacccgacccagtgcgaggccatgaccatggtggccaccaaggtgttcggcaacgacgccacggtcggcttcgccggcagccagggcaacttccagctgaacgtcttcaagccggtcatggcctggtgcgtgctggagtccatccagctgctgggcgacacctgcgtgagcttcaacgaccactgtgcggtgggcattgagcccaacctcgagaagatcaagcacaacctcgacatcaacctgatgcaggtgacggcgctcaaccgccacatcggctacgacaaggcctcgaagatcgccaagaacgcccaccacaagggcattggccttcgtgattcggccctcgagctcggcttcctcacccccgaggagttcgacaagtgggtagtgccggccgatatgacccacccgtccgccgccgacgacgactga

SEQ ID NO: 121 - protein, 468 aa, Propionibacterium acidipropionici
MAEMREEKDSMGTIEVPADHYWGAQTERSLHNFDIGRDTFVWGRDMVRALGTLKKSAALANKELGELPGDVADLIVAAADEVIAGKLDDEFPLVVFQTGSGTQSNMNTNEVISNRAIEIAGGEKGSKTPVHPNDHVNRGQSSNDTFPTAMHIAVVTEINEKLYPAVTQMRNTLDEKAKKFDDVVMVGRTHLQDATPIRLGQVISGWVAQLDFALDGIRYADSRARELAIGGTAVGTGLNAHPKFGETVAKHVSEETGLEFKQAENLFASLSAHDALVQVSGSLRVLGDALMKIANDVRWYASGPRNGIGELLIPENEPGSSIMPGKVNPTQCEAMTMVATRVFGNDATVGFAGSQGNFQLNVFKPVMAHACLESIRLISDACVSFDTHCAYGIEPNMDKINENLDKNLMQVTALNRHIGYDLASKIAKNAHHQGISLRESALTVGGMTAEDFDKWVVPADMTHPSAAE

SEQ ID NO: 122 - DNA, 1407 bp, Propionibacterium acidipropionici
atggcagagatgcgtgaagagaaagacagcatgggcacgatcgaggtgccggccgaccactactggggggcccagaccgagcgttcgctccacaacttcgacatcggacgcgacaccttcgtgtggggtcgcgacatggtccgtgcactgggcaccctgaagaagtcggccgcactggccaacaaggaactgggcgaactgccgggcgacgtcgccgacctcatcgtcgcggccgccgacgaggtcatcgcgggcaagctcgacgacgagttcccgctggtggtcttccagaccgggtccggcacccagtcgaatatgaacaccaacgaggtgatctccaaccgcgccatcgagatcgcgggcggcgagaagggctccaagacccccgtccaccccaacgaccacgtcaaccgcggccagtcctccaacgacaccttccccaccgccatgcacatcgcggtggtcaccgagatcaacgagaagctgtacccggctgtcacgcagatgcgcaacaccctcgacgagaaggccaagaagttcgacgacgtcgtcatggtgggccgcacccacctgcaggacgccaccccgatccgcctgggacaggtcatctccggctgggtcgcccagctggacttcgccctcgacggcatccgctacgccgactcccgggcccgcgagctggccatcggcggcaccgccgtcggcaccggtctcaacgcccacccgaagttcggcgagaccgtcgccaagcacgtctccgaggagaccgggctggagttcaagcaggccgagaacctcttcgcctcgctgagcgcccacgacgccctggtgcaggtgtccggctccctgcgggtgctgggcgacgcgctcatgaagatcgccaacgacgtccgctggtacgcatcgggcccccgcaacggcatcggcgagctgctcatccccgagaacgagcccggctcctcgatcatgcccggcaaggtgaacccgacccagtgcgaggccatgaccatggtcgccacccgggtcttcgggaacgacgccaccgtcggcttcgccggatcccagggcaacttccagctcaacgtgttcaagcccgtcatggcccacgcctgcctggagtcgatccgcctgatctcggacgcctgcgtgtccttcgacacccactgcgcctacggcatcgagccgaacatggacaagatcaacgagaacctggacaagaacctcatgcaggtgaccgccctcaaccgccacatcggttacgacctggcctcgaagatcgccaagaacgcccaccaccagggcatctcgctgcgcgagtcggccctgaccgtcggcgggatgaccgccgaggacttcgacaagtgggtcgtccccgcggacatgacgcacccgtccgcggctgagtga

SEQ ID NO: 123 - protein, 588 aa, Escherichia coli
MKLPVREFDAVVIGAGGAGMRAALQISQSGQTCALLSKVFPTRSHTVSAQGGITVALGNTHEDNWEWHMYDTVKGSDYIGDQDAIEYMCKTGPEAILELEHMGLPFSRLDDGRIYQRPFGGQSKNFGGEQAARTAAAADRTGHALLHTLYQQNLKNHTTIFSEWYALDLVKNQDGAVVGCTALCIETGEVVYFKARATVLATGGAGRIYQSTTNAHINTGDGVGMAIRAGVPVQDMEMWQFHPTGIAGAGVLVTEGCRGEGGYLLNKHGERFMERYAPNAKDLAGRDVVARSIMIEIREGRGCDGPWGPHAKLKLDHLGKEVLESRLPGILELSRTFAHVDPVKEPIPVIPTCHYMMGGIPTKVTGQALTVNEKGEDVVVPGLFAVGEIACVSVHGANRLGGNSLLDLVVFGRAAGLHLQESIAEQGALRDASESDVEASLDRLNRWNNNRNGEDPVAIRKALQECMQHNFSVFREGDAMAKGLEQLKVIRERLKNARLDDTSSEFNTQRVECLELDNLMETAYATAVSANFRTESRGAHSRFDFPDRDDENWLCHSLYLPESESMTRRSVNMEPKLRPAFPPKIRTY

SEQ ID NO: 124 - DNA, 1767 bp, Escherichia coli
atgaaattgccagtcagagaatttgatgcagttgtgattggtgccggtggcgcaggtatgcgcgcggcgctgcaaatttcccagagcggccagacctgtgcgctgctctctaaagtcttcccgacccgttcccataccgtttctgcgcaaggcggcattaccgttgcgctgggtaatacccatgaagataactgggaatggcatatgtacgacaccgtgaaagggtcggactatatcggtgaccaggacgcgattgaatatatgtgtaaaaccgggccggaagcgattctggaactcgaacacatgggcctgccgttctcgcgtctcgatgatggtcgtatctatcaacgtccgtttggcggtcagtcgaaaaacttcggcggcgagcaggcggcacgcactgcggcagcagctgaccgtaccggtcacgcactgttgcacacgctttatcagcagaacctgaaaaaccacaccaccattttctccgagtggtatgcgctggatctggtgaaaaaccaggatggcgcggtggtgggttgtaccgcactgtgcatcgaaaccggtgaagtggtttatttcaaagcccgcgctaccgtgctggcgactggcggagcagggcgtatttatcagtccaccaccaacgcccacattaacaccggcgacggtgtcggcatggctatccgtgccggcgtaccggtgcaggatatggaaatgtggcagttccacccgaccggcattgccggtgcgggcgtactggtcaccgaaggttgccgtggtgaaggcggttatctgctgaacaaacatggcgaacgttttatggagcgttatgcgccgaacgccaaagacctggcgggccgtgacgtggttgcgcgttccatcatgatcgaaatccgtgaaggtcgcggctgtgatggtccgtgggggccacacgcgaaactgaaactcgatcacctgggtaaagaagttctcgaatcccgtctgccgggtatcctggagctttcccgtaccttcgctcacgtcgatccggtgaaagagccgattccggttatcccaacctgtcactacatgatgggcggtattccgaccaaagttaccggtcaggcactgactgtgaatgagaaaggcgaagatgtggttgttccgggactgtttgccgttggtgaaatcgcttgtgtatcggtacacggcgctaaccgtctgggcggcaactcgctgctggacctggtggtctttggtcgcgcggcaggtctgcatctgcaagagtctatcgccgagcagggcgcactgcgcgatgccagcgagtctgatgttgaagcgtctctggatcgcctgaaccgctggaacaataatcgtaacggtgaagatccggtggcgatccgtaaagcgctgcaagaatgtatgcagcataacttctcggtcttccgtgaaggtgatgcgatggcgaaagggcttgagcagttgaaagtgatccgcgagcgtctgaaaaatgcccgtctggatgacacttccagcgagttcaacacccagcgcgttgagtgcctggaactggataacctgatggaaacggcgtatgcaacggctgtttctgccaacttccgtaccgaaagccgtggcgcgcatagccgcttcgacttcccggatcgtgatgatgaaaactggctgtgccactccctgtatctgccagagtcggaatccatgacgcgccgaagcgtcaacatggaaccgaaactgcgcccggcattcccgccgaagattcgtacttactaa

SEQ ID NO: 125 - protein, 238 aa, Escherichia coli
MRLEFSIYRYNPDVDDAPRMQDYTLEADEGRDMMLLDALIQLKEKDPSLSFRRSCREGVCGSDGLNMNGKNGLACITPISALNQPGKKIVIRPLPGLPVIRDLVVDMGQFYAQYEKIKPYLLNNGQNPPAREHLQMPEQREKLDGLYECILCACCSTSCPSFWWNPDKFIGPAGLLAAYRFLIDSRDTETDSRLDGLSDAFSVFRCHSIMNCVSVCPKGLNPTRAIGHIKSMLLQRNA

SEQ ID NO: 126 - DNA, 717 bp, Escherichia coli
atgagactcgagttttcaatttatcgctataacccggatgttgatgatgctccgcgtatgcaggattacaccctggaagcggatgaaggtcgcgacatgatgctgctggatgcgcttatccagctaaaagagaaagatcccagcctgtcgttccgccgctcctgccgtgaaggtgtgtgcggttccgacggtctgaacatgaacggcaagaatggtctggcctgtattaccccgatttcggcactcaaccagccgggcaagaagattgtgattcgcccgctgccaggtttaccggtgatccgcgatttggtggtagacatgggacaattctatgcgcaatatgagaaaattaagccttacctgttgaataatggacaaaatccgccagctcgcgagcatttacagatgccagagcagcgcgaaaaactcgacgggctgtatgaatgtattctctgcgcatgttgttcaacctcttgtccgtctttctggtggaatcccgataagtttatcggcccggcaggcttgttagcggcatatcgtttcctgattgatagccgtgataccgagactgacagccgcctcgacggtttgagtgatgcattcagcgtattccgctgtcacagcatcatgaactgcgtcagtgtatgtccgaaggggctgaacccgacgcgcgccatcggccatatcaagtcgatgttgttgcaacgtaatgcgtaa

SEQ ID NO: 127 - protein, 129 aa, Escherichia coli [sequence omitted]

SEQ ID NO: 128 - DNA, 390 bp, Escherichia coli
atgataagaaatgtgaaaaaacaaagacctgttaatctggacctacagaccatccggttccccatcacggcgatagcgtccattctccatcgcgtttccggtgtgatcacctttgttgcagtgggcatcctgctgtggcttctgggtaccagcctctcttcccctgaaggtttcgagcaagcttccgcgattatgggcagcttcttcgtcaaatttatcatgtggggcatccttaccgctctggcgtatcacgtcgtcgtaggtattcgccacatgatgatggattttggctatctggaagaaacattcgaagcgggtaaacgctccgccaaaatctcctttgttattactgtcgtgctttcacttctcgcaggagtcctcgtatggtaa

SEQ ID NO: 129 - protein, 115 aa, Escherichia coli
MVSNASALGRNGVHDFILVRATAIVLTLYIIYMVGFFATSGELTYEVWIGFFASAFTKVFTLLALFSILIHAWIGMWQVLTDYVKPLALRLMLQLVIVVALVVYVIYGFVVVWGV

SEQ ID NO: 130 - DNA, 348 bp, Escherichia coli
atggtaagcaacgcctccgcattaggacgcaatggcgtacatgatttcatcctcgttcgcgctaccgctatcgtcctgacgctctacatcatttatatggtcggttttttcgctaccagtggcgagctgacatatgaagtctggatcggtttcttcgcctctgcgttcaccaaagtgttcaccctgctggcgctgttttctatcttgatccatgcctggatcggcatgtggcaggtgttgaccgactacgttaaaccgctggctttgcgcctgatgctgcaactggtgattgtcgttgcactggtggtttacgtgatttatggattcgttgtggtgtggggtgtgtga

SEQ ID NO: 131 - protein, 694 aa, Propionibacterium freudenreichii
MNIIKNLFSGAAGKAASTPSAPKPARASAHRPASHLIGEAARDHLGPAQKAAGYEVGAEIDGHVPAGDVLHTWEHRQDDYRLVNPANRRKMKVIVVGSGLSGAGFAASFGQLGYDVDCFCFHDSPRRAHSVAAQGGINAARARKVDGDTLKRFVKDTVKGGDYRGREADVVRLGTESVRVIDHMYAIGAPFAREYGGQLATRSFGGVQVSRTYYTRGETGQQMEIACSQALQEQIDAGTVKMHNRTEMLDLIVKDGRAQGIVTRDLLTGEIKAWTAHVVVLCTGGYGSVYHWSTLAKNSNATATWRAHKQGAYFASPCFLQFHPTALPVSSHWQSKTTLMSESLRNDGRIWVPKKAGDDRPANDIPENERDYYLERKYPAFGNLTPRDVASRNARTQIDSGHGVGPLHNSVYLDFRDAIKRLGKETIAERYGNLFDMYLDATGENPYEVPMRIAPGAHFSMGGLWVDYDQMSNLPGLFVGGEASNNYHGANRLGANSLLSASVDGWFTLPLSVPNYLADYVGKPPLAVQDPAVKDALGRVQDRINAFLTSKGTHRPEWFHRKLGDILYAYCGVSRDEAGLTKGLAEVRALRKEYWNDVKVVGDDHRLNQELEKAGRVADFIELAEVMILDALDRRESAGAHFRTEYATPEGEAKRNDADWCAVSAWETRPDGVHVRHSEPLEFSLIDLQVRDYR

SEQ ID NO: 132 - DNA, 2085 bp, Propionibacterium freudenreichii
gtgaatatcatcaagaatctcttctccggtgcggccggcaaggctgcatcgaccccgtcagccccgaagcctgcccgtgccagtgcgcaccgtccggcctcgcacctgatcggtgaggccgcccgcgaccacctgggcccggcccagaaggccgccggctatgaggtcggtgccgagatcgacgggcacgtccccgccggcgatgtgctccacacctgggagcaccgtcaggacgactaccgactagtcaacccggccaaccgtcgcaagatgaaggtcatcgtcgtgggctccggcctgtccggtgcgggcttcgcggccagcttcggccagctcggctatgacgtcgactgcttctgtttccatgattcgccgcgtcgcgcccactccgtggcggcgcagggcggcatcaacgccgctcgtgcccgcaaggtcgacggtgacacgctgaagcgcttcgtcaaggacaccgtcaagggcggcgactaccggggccgtgaggccgacgtggtgcgccttggtacggagtcggtgcgtgtcatcgaccacatgtacgcgatcggtgcccccttcgcccgtgaatacggcggtcagctcgccacccgttccttcggtggcgtgcaggtctcgcgtacctattacacgcgcggcgagaccggccagcagatggagatcgcctgttcccaggcgctccaggagcagatcgacgccggcaccgtgaagatgcacaaccgcaccgagatgcttgacctgatcgtcaaggacggccgtgcccagggcatcgtcacccgcgatctgctgaccggcgagatcaaggcctggacggcccatgtcgtggtgctgtgcaccggcggctacggctcggtctaccactggtccacgctggccaagaactcgaatgcaaccgccacctggcgtgcgcacaagcagggcgcgtacttcgcgagcccgtgcttcctgcagttccaccccacggcgcttccggtcagttcacactggcagtcgaagaccacgctgatgagtgagtccctgcgcaatgacggacgcatctgggtgccgaagaaggccggcgacgatcgcccggccaatgacatccccgagaacgagcgcgactactacctggagcgcaagtacccggcattcggcaacctgacgccccgtgacgtcgccagccgcaacgcccgcacgcagattgacagcgggcacggcgtggggccgctgcacaactcggtgtacctcgacttccgcgacgccatcaagcgtctcggcaaggagaccatcgccgagcgctacggcaacctgttcgacatgtacctcgacgccaccggtgagaacccctatgaggtgcccatgcgcatcgcaccgggtgcccacttctcgatgggtggcctgtgggtcgactacgaccagatgagcaacctgcccggtctgttcgtcggcggagaggcatcgaacaactaccacggtgcgaaccgcctgggtgccaactccctgttgtccgcctccgtggatggctggttcaccctgccgctgtcggtgccgaactacctcgccgactatgtcggcaagccgccgctggccgtgcaggatccggccgtcaaggatgccctgggccgggtgcaggatcgcatcaatgccttcctcaccagcaagggcacgcatcgtcccgagtggttccatcgcaagcttggcgacatcctctacgcctactgtggcgtgagccgtgacgaggcgggcctgaccaagggcctcgccgaggtgcgggcactgcgcaaggagtactggaacgacgtcaaggtcgtcggcgacgaccaccggctcaaccaggaactcgagaaggccggccgcgtggccgacttcatcgagctcgccgaggtcatgatcctcgacgccctggaccgccgcgagtcggccggtgcccacttccgtaccgagtacgccactcccgagggagaggccaagcgcaacgacgccgattggtgcgccgtctcggcctgggagacccgccccgatggggttcatgtccgtcacagcgagcccctggaattctcgctgatcgatctgcaggtgagggattaccgatga

SEQ ID NO: 133 - protein, 673 aa, Propionibacterium freudenreichii
MTENNVAGLPAGDEKYYTLGAEVHDTKANTDVPIEKIWPDRQFNAKLVNPANRRKMTVIVVGTGLAGGAAAASLGEAGYHVENFCYQDSPRRAHSIAAQGGINAAKDYKNDNDSVYRLFYDTVKGGDYRARETNVYRLADVSSNIIDQCVAVGVPFAREYGGLLDNRSFGGVQVQRTFYAKGQTGQQLLIGVYQSLSRQVAAGTVHMHSRHEMLELIVKDGHARGIVTRDMVTGKIESWTADAVVIASGGYGNVFFLSTNAMGCNTTATWRAHRKGAYFANPCYTQIHPTCIPVHGENQSKLTLMSESLRNDGRIWVPKRAEDADKDPRQIPEADRDYYLERIYPSFGNLVPRDIASRQAKNMCDEGRGVGPAIKEIGPDGKERMVRRGVYLDFSDAIERLGKRGVAAKYGNLFDMYKQITAEDPYETPMRIYPAVHYTMGGLWVDYDLESSIPGLYVAGEANFSDHGANRLGASALMQGLSDGYFVLPNTMNDYLAGNHEGKLPDDDPAVVEAVASVKDRVNKLLAVKGERTVDSFHKELGQIMWEYCGMARSEEGLKTAIDKIRVLRDEFWKNVKVTGVNEDLNQTLERAGRVADFLELGELMCIDAMHRRESCGGHFRVESQTPEGEALRDDKDFLYVAAWEFTGDGQKPELHKEPLVYKAIELKQRSYK

SEQ ID NO: 134 - DNA, 2022 bp, Propionibacterium freudenreichii
gtgactgagaacaatgtggcgggccttcccgccggcgatgagaagtactacacgctgggcgccgaggtgcatgacaccaaggcgaacaccgatgttccgatcgagaagatctggcccgaccgccagttcaatgccaagctcgtgaaccctgccaaccgccgcaagatgacagtgatcgtcgtcggcaccgggttggctggcggcgctgccgctgcctcactgggcgaggccggctaccacgtcgagaacttctgctaccaggacagccctcgtcgtgcgcactcgattgcggcccagggcggcatcaacgctgccaaggactacaagaacgacaacgactccgtctaccggctgttctatgacacggtcaagggcggcgactaccgagcccgcgagaccaacgtctaccggttggccgacgtgagctccaacatcatcgaccagtgcgtcgccgtgggcgttcccttcgcccgcgagtacggcggcctgctcgacaaccgttcgttcggtggcgtccaggtgcagcgcaccttctacgccaagggccagacgggtcagcagctgctgatcggcgtctaccagtccctgtcgcgtcaggtggcagccggcacggtgcacatgcacagccgccacgagatgctcgaactgatcgtcaaggacggtcatgcccgcggcatcgtgacgcgcgacatggtcaccggcaagatcgagtcgtggactgctgacgcagtggtcatcgcctctggtggctacggcaatgtgttcttcctgtcgaccaatgccatgggatgcaacaccacggcgacgtggcgtgcgcaccgcaagggcgcctacttcgccaacccgtgctacacgcagatccacccgacctgtatccccgtgcacggcgagaaccagtccaagctcaccctgatgagtgagtcgttgcgcaacgacggtcgtatctgggtgcccaagcgcgccgaggacgccgacaaggatccgcgtcagatccccgaggcggatcgcgactactacctcgagcgcatctatccctcattcggaaacctggtgccccgcgacatcgcatcgcgccaggccaagaacatgtgtgacgagggccgcggtgtcggtccggcgatcaaggagatcggtccggacggcaaggagcggatggttcgtcgtggcgtctacctcgacttctccgacgccatcgagcgtctgggcaagcggggagtggccgccaagtacggcaacttgttcgacatgtacaagcagatcaccgcggaggatccctatgagacccccatgcgcatctaccccgccgtgcactacacgatgggtggcctgtgggtcgactacgacctggagagctcgatccccggcctgtacgtcgccggcgaggcgaacttctccgaccacggtgccaaccgactcggtgcctcggccctgatgcagggcctgtccgacgggtacttcgtgctcccgaacaccatgaacgactacctggccggcaatcacgagggcaagctgcccgacgatgatccggcagtggtcgaggccgttgcctccgtgaaggaccgcgtcaacaagctgctggccgtcaagggcgagcgcacggttgactcgttccacaaggaactcggccagatcatgtgggagtactgcggcatggcccgcagcgaggagggcctcaagacggccatcgacaagatccgcgtgctgcgtgacgagttctggaagaacgtcaaggtgaccggtgtcaacgaggatctgaaccagaccctcgagcgcgccggtcgcgtggctgacttcctggagctcggtgaactgatgtgcatcgatgccatgcatcgccgcgagagctgtggcggtcacttccgggttgagtcccagactccggagggcgaggccctgcgtgacgacaaggacttcctctacgtcgccgcttgggagttcaccggcgacgggcagaagcccgagctgcacaaggagccgctggtctacaaggcaatcgaactcaagcaacggagttacaagtga

SEQ ID NO: 135 - protein, 252 aa, Propionibacterium freudenreichii
MKVTLDIWRQAGPRAKGEFENYVVNDAEPEMSILELLDRLNDQIIEQGGEPVVFESDCREGVCGCCGFLVNGKPHGPLANTPACRQHLRAFPEVTHFKLEPFRSNAFPVIRDLAIDRTALDELIQAGGTVNVMTGTAPDADTSPQPHQVAELALDFASCIGCGACVAACPNGSAMLFAGAKLAHLAKMPQGKEQRSSRARRMVAELDEDFGPCSLYGECAISCPAGISLTAIATVNKERWRSVFRGRHSQDN

SEQ ID NO: 136 - DNA, 759 bp, Propionibacterium freudenreichii
atgaaggtcacattggatatctggcgccaggcaggtcctcgcgccaagggtgagttcgaaaactacgtcgtcaacgacgctgagcccgagatgagcatccttgagttgctcgatcgactcaacgaccagatcatcgaacagggcggcgagcccgtcgtcttcgagtctgattgtcgtgagggcgtgtgtgggtgctgtggcttcctggtcaatgggaagccccacggtccgctggccaatacgccggcctgtcgccagcacctgcgcgccttccccgaggtgacgcacttcaagttggagcccttccgctccaatgcgttcccggtgatccgcgacctggcgatcgaccgcaccgccctggatgagctcatccaggccggcggcaccgtcaacgtgatgaccggcaccgctccggacgccgacaccagcccccagccgcaccaggtggccgagctcgcgctcgacttcgccagctgcatcggctgcggagcctgcgtggccgcctgcccgaatggttcggcgatgctgttcgccggcgccaagctggcgcatctggcgaagatgccccagggcaaggagcagcgcagctcgagggcgcgtcgcatggtggcagagctcgatgaggacttcggtccctgctcgctgtacggcgagtgcgccatctcctgcccggccggcatctcgctgaccgcgatcgccaccgtgaacaaggagcgctggcgttccgtgttccgcgggcgccactcgcaggacaactga

SEQ ID NO: 137 - protein, 254 aa, Propionibacterium freudenreichii
MKLTLNIWRQAEGADNGAIKTYQIDGISGDTSFLEMLDELNEQLTHQGEEPVAFDSDCREGICGMCGVVINGTAHGRGVSPVRTTTCQLHMRSFKDGDTITIEPWKTPVFPVIKDLVVDRSALDRVVQAGGFISVNTGAAPDAHSTPAPKRQADRAFDNATCIGCGACVAACPHSSAMLFTSAKITHLANLPQGQPERKLRVKNMVGQHDAEGFGHCTNIGECAAVCPKGIPLESINQLNRDLIASLFSGDGKE

SEQ ID NO: 138 - DNA, 765 bp, Propionibacterium freudenreichii
gtgaagctgacactgaatatctggcgtcaggccgagggtgccgacaacggcgccatcaagacctatcaaatcgatggaatcagcggcgacacctcgttcctcgagatgcttgatgagctcaacgagcagttgacccatcagggcgaggagcccgtggcgttcgattccgattgtcgtgaaggcatctgcggaatgtgtggcgtggtcatcaatggcaccgcccacggccgcggcgtgtccccggtgcgcaccacgacgtgccagctgcacatgcgttcgttcaaggatggcgacacgatcacgatcgagccgtggaagacccccgtcttcccggtcatcaaggacctggtcgtcgaccgcagtgcgctggaccgcgtggtccaggccggtggcttcatctccgtgaacaccggtgctgcccccgacgcgcattccacgccggctccgaagcgccaggccgatcgggccttcgacaacgcgacctgcattggttgtggcgcgtgcgtggcagcctgcccgcacagctccgccatgctcttcacgtcggcgaagatcacccacctggcgaacctgccccagggccagcccgagcgcaagctgcgggtcaagaacatggtcggtcaacacgacgcagagggcttcgggcactgcaccaacatcggtgagtgcgcggccgtctgcccgaagggcatcccgctcgagtcgatcaaccagctgaaccgtgacctcattgcgtcgctgttctccggtgacggcaaggagtga

SEQ ID NO: 139 - protein, 246 aa, Propionibacterium freudenreichii
MSVGLTTSGGQGDVVTRHKLKQRPSNVTLKVTMAVTGTIFALFVFVHMVGNLKAFMGPEDYDAYARFLRTLLYPLLPYEGGLWIFRLVLSACLVLHVWAGITWLRGRKARGKFGRYGAKPKSFFARTMILSGLLILVFVVVHLLDLTIGAGLSSQYYQPAVHLGGDQVQIHAYENLVASLSRPWMAIFYSVIMVIIGCHIGQGAWNTINDFGGTGPRLRKVWFLIGLLIALAIVVANGALPMLILA

SEQ ID NO: 140 - DNA, 756 bp, Propionibacterium freudenreichii
atgagtgtcggtctgacgacatcaggagggcaaggggacgttgtaacgcgtcacaagctgaagcaacgtccgtccaatgtgactctgaaggtcaccatggcggtgactggaacgatcttcgccctgttcgtctttgtgcacatggtcggaaatctcaaggcttttatgggccctgaagattacgacgcctacgcccgcttcctgcgcaccctgctgtatccgctgctgccctatgagggtggtctgtggatcttccgcctggtgctgtcagcctgcctggtgctgcacgtctgggccggcattaccgtctggctgcgtggccgtaaggctcgtggcaagttcggtcgttacggcgccaagcccaagtccttcttcgctcgcacgatgatcctgtcgggcctgctgatcctggtcttcgtggtggtccacctgctcgatctcacgatcggcgccggactgtcctcgcagtactaccagcctgccgtccacctcggtggcgaccaggtccagatccatgcctacgagaacctcgtggccagcctgtcccgtccctggatggcgatcttctactccgtgatcatggtgatcatcggatgccatatcggccagggtgcctggaacacgatcaatgacttcggtggcaccggcccccgtcttcgcaaggtctggttcctcatcgggctcctcattgcgctggccatcgtcgtggccaacggtgcactccccatgctcatcctcgctggagtgatctcgtga

SEQ ID NO: 141 - protein, 247 aa, Propionibacterium freudenreichii
MATLTTQSADTTPQSRALRSTVARKFLMALTGIFLVAFLAMHMFGNLKLLMNDSGAEFDAYSHALRQFLVPILPPYFFLTLFRIVLGAAVIIHMGLAIDLTLRDRKASGVGFKRYVQRRYLEGSFAARTMIWGGIIIALFLVFHLLQFTDQIIKVGYSAGDPAVDQPHLRVILGFQNWGIYAIYFVAMLAVCLHIWHGFRSAFSTLGWRVGNSSTVVIKVCAWLVSILVFVGFMLVPTLIAFGVITQ

SEQ ID NO: 142 - DNA, 744 bp, Propionibacterium freudenreichii
gtggcaactctcactactcaatcagcggacacgactccgcagagcagggcgctgcgctccacggtggccagaaaattcctgatggctctcaccggtatcttcttggtggcgttcctggcgatgcacatgttcggcaatctgaagctgctcatgaacgattcaggcgccgagttcgacgcctactcgcatgcgctgcggcagttccttgtgccgatcctcccgccctacttcttccttacgctcttccgcatcgtgctcggtgccgcagtcatcattcacatgggactggccatcgacctcacgctgcgtgaccgcaaggcgagtggtgtgggcttcaagcggtatgtccagcgtcgctacctcgaaggcagcttcgcggcccgcaccatgatctggggcggcattatcatcgccctgttcctggtgttccacctgctgcagttcaccgaccagatcatcaaggttggctactcggcgggcgatccggccgtcgaccagccgcacctgcgcgtgatcctcggcttccagaactggggcatctatgcgatctacttcgtggccatgctggccgtctgcctccatatctggcacggtttccgctcggccttcagcacactcggttggcgtgtcggcaactcgtcgacggtcgtcatcaaggtctgcgcgtggctcgtgtcgatcctggtgttcgtgggcttcatgctcgtcccgacccttattgcctttggagtgatcacccagtga

SEQ ID NO: 143 - protein, 289 aa, Escherichia coli
MSILIDKNTKVICQGFTGSQGTFHSEQAIAYGTKMVGGVTPGKGGTTHLGLPVFNTVREAVAATGATASVIYVPAPFCKDSILEAIDAGIKLIITITEGIPTLDMLTVKVKLDEAGVRMIGPNCPGVITPGECKIGIQPGHIHKPGKVGIVSRSGTLTYEAVKQTTDYGFGQSTCVGIGGDPIPGSNFIDILEMFEKDPQTEAIVMIGEIGGSAEEEAAAYIKEHVTKPVVGYIAGVTAPKGKRMGHAGAIIAGGKGTADEKFAALEAAGVKTVRSLADIGEALKTVLK

SEQ ID NO: 144 - DNA, 870 bp, Escherichia coli
atgtccattttaatcgataaaaacaccaaggttatctgccagggctttaccggtagccaggggactttccactcagaacaggccattgcatacggcactaaaatggttggcggcgtaaccccaggtaaaggcggcaccacccacctcggcctgccggtgttcaacaccgtgcgtgaagccgttgctgccactggcgctaccgcttctgttatctacgtaccagcaccgttctgcaaagactccattctggaagccatcgacgcaggcatcaaactgattatcaccatcactgaaggcatcccgacgctggatatgctgaccgtgaaagtgaagctggatgaagcaggcgttcgtatgatcggcccgaactgcccaggcgttatcactccgggtgaatgcaaaatcggtatccagcctggtcacattcacaaaccgggtaaagtgggtatcgtttcccgttccggtacactgacctatgaagcggttaaacagaccacggattacggtttcggtcagtcgacctgtgtcggtatcggcggtgacccgatcccgggctctaactttatcgacattctcgaaatgttcgaaaaagatccgcagaccgaagcgatcgtgatgatcggtgagatcggcggtagcgctgaagaagaagcagctgcgtacatcaaagagcacgttaccaagccagttgtgggttacatcgctggtgtgactgcgccgaaaggcaaacgtatgggccacgcgggtgccatcattgccggtgggaaagggactgcggatgagaaattcgctgctctggaagccgcaggcgtgaaaaccgttcgcagcctggcggatatcggtgaagcactgaaaactgttctgaaataa

SEQ ID NO: 145 - protein, 388 aa, Escherichia coli
MNLHEYQAKQLFARYGLPAPVGYACTTPREAEEAASKIGAGPWVVKCQVHAGGRGKAGGVKVVNSKEDIRAFAENWLGKRLVTYQTDANGQPVNQILVEAATDIAKELYLGAVVDRSSRRVVFMASTEGGVEIEKVAEETPHLIHKVALDPLTGPMPYQGRELAFKLGLEGKLVQQFTKIFMGLATIFLERDLALIEINPLVITKQGDLICLDGKLGADGNALFRQPDLREMRDQSQEDPREAQAAQWELNYVALDGNIGCMVNGAGLAMGTMDIVKLHGGEPANFLDVGGGATKERVTEAFKIILSDDKVKAVLVNIFGGIVRCDLIADGIIGAVAEVGVNVPVVVRLEGNNAELGAKKLADSGLNIIAAKGLTDAAQQVVAAVEGK

SEQ ID NO: 146 - DNA, 1167 bp, Escherichia coli
atgaacttacatgaatatcaggcaaaacaactttttgcccgctatggcttaccagcaccggtgggttatgcctgtactactccgcgcgaagcagaagaagccgcttcaaaaatcggtgccggtccgtgggtagtgaaatgtcaggttcacgctggtggccgcggtaaagcgggcggtgtgaaagttgtaaacagcaaagaagacatccgtgcttttgcagaaaactggctgggcaagcgtctggtaacgtatcaaacagatgccaatggccaaccggttaaccagattctggttgaagcagcgaccgatatcgctaaagagctgtatctcggtgccgttgttgaccgtagttcccgtcgtgtggtctttatggcctccaccgaaggcggcgtggaaatcgaaaaagtggcggaagaaactccgcacctgatccataaagttgcgcttgatccgctgactggcccgatgccgtatcagggacgcgagctggcgttcaaactgggtctggaaggtaaactggttcagcagttcaccaaaatcttcatgggcctggcgaccattttcctggagcgcgacctggcgttgatcgaaatcaacccgctggtcatcaccaaacagggcgatctgatttgcctcgacggcaaactgggcgctgacggcaacgcactgttccgccagcctgatctgcgcgaaatgcgtgaccagtcgcaggaagatccgcgtgaagcacaggctgcacagtgggaactgaactacgttgcgctggacggtaacatcggttgtatggttaacggcgcaggtctggcgatgggtacgatggacatcgttaaactgcacggcggcgaaccggctaacttccttgacgttggcggcggcgcaaccaaagaacgtgtaaccgaagcgttcaaaatcatcctctctgacgacaaagtgaaagccgttctggttaacatcttcggcggtatcgttcgttgcgacctgatcgctgacggtatcatcggcgcggtagcagaagtgggtgttaacgtaccggtcgtggtacgtctggaaggtaacaacgccgaactcggcgcgaagaaactggctgacagcggcctgaatattattgcagcaaaaggtctgacggatgcagctcagcaggttgttgccgcagtggaggggaaataa

SEQ ID NO: 147 - protein, 540 aa, Escherichia coli
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 148 - DNA, 1623 bp, Escherichia coli
atgcgcgttaacaatggtttgaccccgcaagaactcgaggcttatggtatcagtgacgtacatgatatcgtttacaacccaagctacgacctgctgtatcaggaagagctcgatccgagcctgacaggttatgagcgcggggtgttaactaatctgggtgccgttgccgtcgataccgggatcttcaccggtcgttcaccaaaagataagtatatcgtccgtgacgataccactcgcgatactttctggtgggcagacaaaggcaaaggtaagaacgacaacaaacctctctctccggaaacctggcagcatctgaaaggcctggtgaccaggcagctttccggcaaacgtctgttcgttgtcgacgctttctgtggtgcgaacccggatactcgtctttccgtccgtttcatcaccgaagtggcctggcaggcgcattttgtcaaaaacatgtttattcgcccgagcgatgaagaactggcaggtttcaaaccagactttatcgttatgaacggcgcgaagtgcactaacccgcagtggaaagaacagggtctcaactccgaaaacttcgtggcgtttaacctgaccgagcgcatgcagctgattggcggcacctggtacggcggcgaaatgaagaaagggatgttctcgatgatgaactacctgctgccgctgaaaggtatcgcttctatgcactgctccgccaacgttggtgagaaaggcgatgttgcggtgttcttcggcctttccggcaccggtaaaaccaccctttccaccgacccgaaacgtcgcctgattggcgatgacgaacacggctgggacgatgacggcgtgtttaacttcgaaggcggctgctacgcaaaaactatcaagctgtcgaaagaagcggaacctgaaatctacaacgctatccgtcgtgatgcgttgctggaaaacgtcaccgtgcgtgaagatggcactatcgactttgatgatggttcaaaaaccgagaacacccgcgtttcttatccgatctatcacatcgataacattgttaagccggtttccaaagcgggccacgcgactaaggttatcttcctgactgctgatgctttcggcgtgttgccgccggtttctcgcctgactgccgatcaaacccagtatcacttcctctctggcttcaccgccaaactggccggtactgagcgtggcatcaccgaaccgacgccaaccttctccgcttgcttcggcgcggcattcctgtcgctgcacccgactcagtacgcagaagtgctggtgaaacgtatgcaggcggcgggcgcgcaggcttatctggttaacactggctggaacggcactggcaaacgtatctcgattaaagatacccgcgccattatcgacgccatcctcaacggttcgctggataatgcagaaaccttcactctgccgatgtttaacctggcgatcccaaccgaactgccgggcgtagacacgaagattctcgatccgcgtaacacctacgcttctccggaacagtggcaggaaaaagccgaaaccctggcgaaactgtttatcgacaacttcgataaatacaccgacacccctgcgggtgccgcgctggtagcggctggtccgaaactgtaa

SEQ ID NO: 149 - protein, 470 aa, Escherichia coli
MKKTKIVCTIGPKTESEEMLAKMLDAGMNVMRLNFSHGDYAEHGQRIQNLRNVMSKTGKTAAILLDTKGPEIRTMKLEGGNDVSLKAGQTFTFTTDKSVIGNSEMVAVTYEGFTTDLSVGNTVLVDDGLIGMEVTAIEGNKVICKVLNNGDLGENKGVNLPGVSIALPALAEKDKQDLIFGCEQGVDFVAASFIRKRSDVIEIREHLKAHGGENIHIISKIENQEGLNNFDEILEASDGIMVARGDLGVEIPVEEVIFAQKMMIEKCIRARKVVITATQMLDSMIKNPRPTRAEAGDVANAILDGTDAVMLSGESAKGKYPLEAVSIMATICERTDRVMNSRLEFNNDNRKLRITEAVCRGAVETAEKLDAPLIVVATQGGKSARAVRKYFPDATILALTTNEKTAHQLVLSKGVVPQLVKEITSTDDFYRLGKELALQSGLAHKGDVVVMVSGALVPSGTTNTASVHVL

SEQ ID NO: 150 - DNA, 1413 bp, Escherichia coli
atgaaaaagaccaaaattgtttgcaccatcggaccgaaaaccgaatctgaagagatgttagctaaaatgctggacgctggcatgaacgttatgcgtctgaacttctctcatggtgactatgcagaacacggtcagcgcattcagaatctgcgcaacgtgatgagcaaaactggtaaaaccgccgctatcctgcttgataccaaaggtccggaaatccgcaccatgaaactggaaggcggtaacgacgtttctctgaaagctggtcagacctttactttcaccactgataaatctgttatcggcaacagcgaaatggttgcggtaacgtatgaaggtttcactactgacctgtctgttggcaacaccgtactggttgacgatggtctgatcggtatggaagttaccgccattgaaggtaacaaagttatctgtaaagtgctgaacaacggtgacctgggcgaaaacaaaggtgtgaacctgcctggcgtttccattgctctgccagcactggctgaaaaagacaaacaggacctgatctttggttgcgaacaaggcgtagactttgttgctgcttcctttattcgtaagcgttctgacgttatcgaaatccgtgagcacctgaaagcgcacggcggcgaaaacatccacatcatctccaaaatcgaaaaccaggaaggcctcaacaacttcgacgaaatcctcgaagcctctgacggcatcatggttgcgcgtggcgacctgggtgtagaaatcccggtagaagaagttatcttcgcccagaagatgatgatcgaaaaatgtatccgtgcacgtaaagtcgttatcactgcgacccagatgctggattccatgatcaaaaacccacgcccgactcgcgcagaagccggtgacgttgcaaacgccatcctcgacggtactgacgcagtgatgctgtctggtgaatccgcaaaaggtaaatacccgctggaagcggtttctatcatggcgaccatctgcgaacgtaccgaccgcgtgatgaacagccgtctcgagttcaacaatgacaaccgtaaactgcgcattaccgaagcggtatgccgtggtgccgttgaaactgctgaaaaactggatgctccgctgatcgtggttgctactcagggcggtaaatctgctcgcgcagtacgtaaatacttcccggatgccaccatcctggcactgaccaccaacgaaaaaacggctcatcagttggtactgagcaaaggcgttgtgccgcagcttgttaaagagatcacttctactgatgatttctaccgtctgggtaaagaactggctctgcagagcggtctggcacacaaaggtgacgttgtagttatggtttctggtgcactggtaccgagcggcactactaacaccgcatctgttcacgtcctgtaa

SEQ ID NO: 151 - protein, 480 aa, Escherichia coli
MSRRLRRTKIVTTLGPATDRDNNLEKVIAAGANVVRMNFSHGSPEDHKMRADKVREIAAKLGRHVAILGDLQGPKIRVSTFKEGKVFLNIGDKFLLDANLGKGEGDKEKVGIDYKGLPADVVPGDILLLDDGRVQLKVLEVQGMKVFTEVTVGGPLSNNKGINKLGGGLSAEALTEKDKADIKTAALIGVDYLAVSFPRCGEDLNYARRLARDAGCDAKIVAKVERAEAVCSQDAMDDIILASDVVMVARGDLGVEIGDPELVGIQKALIRRARQLNRAVITATQMMESMITNPMPTRAEVMDVANAVLDGTDAVMLSAETAAGQYPSETVAAMARVCLGAEKIPSINVSKHRLDVQFDNVEEAIAMSAMYAANHLKGVTAIITMTESGRTALMTSRISSGLPIFAMSRHERTLNLTALYRGVTPVHFDSANDGVAAASEAVNLLRDKGYLMSGDLVIVTQGDVMSTVGSTNTTRILTVE

SEQ ID NO: 152 - DNA, 1443 bp, Escherichia coli
atgtccagaaggcttcgcagaacaaaaatcgttaccacgttaggcccagcaacagatcgcgataataatcttgaaaaagttatcgcggcgggtgccaacgttgtacgtatgaacttttctcacggctcgcctgaagatcacaaaatgcgcgcggataaagttcgtgagattgccgcaaaactggggcgtcatgtggctattctgggtgacctccaggggcccaaaatccgtgtatccacctttaaagaaggcaaagttttcctcaatattggggataaattcctgctcgacgccaacctgggtaaaggtgaaggcgacaaagaaaaagtcggtatcgactacaaaggcctgcctgctgacgtcgtgcctggtgacatcctgctgctggacgatggtcgcgtccagttaaaagtactggaagttcagggcatgaaagtgttcaccgaagtcaccgtcggtggtcccctctccaacaataaaggtatcaacaaacttggcggcggtttgtcggctgaagcgctgaccgaaaaagacaaagcagacattaagactgcggcgttgattggcgtagattacctggctgtctccttcccacgctgtggcgaagatctgaactatgcccgtcgcctggcacgcgatgcaggatgtgatgcgaaaattgttgccaaggttgaacgtgcggaagccgtttgcagccaggatgcaatggatgacatcatcctcgcctctgacgtggtaatggttgcacgtggcgacctcggtgtggaaattggcgacccggaactggtcggcattcagaaagcgttgatccgtcgtgcgcgtcagctaaaccgagcggtaatcacggcgacccagatgatggagtcaatgattactaacccgatgccgacgcgtgcagaagtcatggacgtagcaaacgccgttctggatggtactgacgctgtgatgctgtctgcagaaactgccgctgggcagtatccgtcagaaaccgttgcagccatggcgcgcgtttgcctgggtgcggaaaaaatcccgagcatcaacgtttctaaacaccgtctggacgttcagttcgacaatgtggaagaagctattgccatgtcagcaatgtacgcagctaaccacctgaaaggcgttacggcgatcatcaccatgaccgaatcgggtcgtaccgcgctgatgacctcccgtatcagctctggtctgccaattttcgccatgtcgcgccatgaacgtacgctgaacctgactgctctctatcgtggcgttacgccggtgcactttgatagcgctaatgacggcgtagcagctgccagcgaagcggttaatctgctgcgcgataaaggttacttgatgtctggtgacctggtgattgtcacccagggcgacgtgatgagtaccgtgggttctactaataccacgcgtattttaacggtagagtaa

SEQ ID NO: 153 - protein, 427 aa, Escherichia coli
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 154 - DNA, 1284 bp, Escherichia coli
atggctgatacaaaagcaaaactcaccctcaacggggatacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccatgctttccgtcgcgactcgcatccaatggcagtcatgtgtggtattaccggcgcgctggcggcgttctatcacgactcgctggatgttaacaatcctcgtcaccgtgaaattgccgcgttccgcctgctgtcgaaaatgccgaccatggccgcgatgtgttacaagtattccattggtcagccatttgtttacccgcgcaacgatctctcctacgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggcggtgctaacgaagcggcgctgaaaatgctggaagaaatcagctccgttaaacacattccggaatttgttcgtcgtgcgaaagacaaaaatgattctttccgcctgatgggcttcggtcaccgcgtgtacaaaaattacgacccgcgcgccaccgtaatgcgtgaaacctgccatgaagtgctgaaagagctgggcacgaaggatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaa

SEQ ID NO: 155 - protein, 865 aa, Escherichia coli
MLEEYRKHVAERAAEGIAPKPLDANQMAALVELLKNPPAGEEEFLLDLLTNRVPPGVDEAAYVKAGFLAAIAKGEAKSPLLTPEKAIELLGTMQGGYNIHPLIDALDDAKLAPIAAKALSHTLLMFDNFYDVEEKAKAGNEYAKQVMQSWADAEWFLNRPALAEKLTVTVFKVTGETNTDDLSPAPDAWSRPDIPLHALAMLKNAREGIEPDQPGVVGPIKQIEALQQKGFPLAYVGDVVGTGSSRKSATNSVLWFMGDDIPHVPNKRGGGLCLGGKIAPIFFNTMEDAGALPIEVDVSNLNMGDVIDVYPYKGEVRNHETGELLATFELKTDVLIDEVRAGGRIPLIIGRGLTTKAREALGLPHSDVFRQAKDVAESDRGFSLAQKMVGRACGVKGIRPGAYCEPKMTSVGSQDTTGPMTRDELKDLACLGFSADLVMQSFCHTAAYPKPVDVNTHHTLPDFIMNRGGVSLRPGDGVIHSWLNRMLLPDTVGTGGDSHTRFPIGISFPAGSGLVAFAAATGVMPLDMPESVLVRFKGKMQPGITLRDLVHAIPLYAIKQGLLTVEKKGKKNIFSGRILEIEGLPDLKVEQAFELTDASAERSAAGCTIKLNKEPIIEYLNSNIVLLKWMIAEGYGDRRTLERRIQGMEKWLANPELLEADADAEYAAVIDIDLADIKEPILCAPNDPDDARPLSAVQGEKIDEVFIGSCMTNIGHFRAAGKLLDAHKGQLPTRLWVAPPTRMDAAQLTEEGYYSVFGKSGARIEIPGCSLCMGNQARVADGATVVSTSTRNFPNRLGTGANVFLASAELAAVAALIGKLPTPEEYQTYVAQVDKTAVDTYRYLNFNQLSQYTEKADGVIFQTAV

SEQ ID NO: 156 - DNA, 2598 bp, Escherichia coli
gtgctagaagaataccgtaagcacgtagctgagcgtgccgctgaggggattgcgcccaaacccctggatgcaaaccaaatggccgcacttgtagagctgctgaaaaacccgcccgcgggcgaagaagaattcctgttagatctgttaaccaaccgtgttcccccaggcgtcgatgaagccgcctatgtcaaagcaggcttcctggctgctatcgcgaaaggcgaagccaaatcccctctgctgactccggaaaaagccatcgaactgctgggcaccatgcagggtggttacaacattcatccgctgatcgacgcgctggatgatgccaaactggcacctattgctgccaaagcactttctcacacgctgctgatgttcgataacttctatgacgtagaagagaaagcgaaagcaggcaacgaatatgcgaagcaggttatgcagtcctgggcggatgccgaatggttcctgaatcgcccggcgctggctgaaaaactgaccgttactgtcttcaaagtcactggcgaaactaacaccgatgacctttctccggcaccggatgcgtggtcacgcccggatatcccactgcacgcgctggcgatgctgaaaaacgcccgtgaaggtattgagccagaccagcctggtgttgttggtccgatcaagcaaatcgaagctctgcaacagaaaggtttcccgctggcgtacgtcggtgacgttgtgggtacgggttcttcgcgtaaatccgccactaactccgttctgtggtttatgggcgatgatattccacatgtgccgaacaaacgcggcggtggtttgtgcctcggcggtaaaattgcacccatcttctttaacacgatggaagacgcgggtgcactgccaatcgaagtcgacgtctctaacctgaacatgggcgacgtgattgacgtttacccgtacaaaggtgaagtgcgtaaccacgaaaccggcgaactgctggcgaccttcgaactgaaaaccgacgtgctgattgatgaagtgcgtgctggtggccgtattccgctgattatcgggcgtggcctgaccaccaaagcgcgtgaagcacttggtctgccgcacagtgatgtgttccgtcaggcgaaagatgtcgctgagagcgatcgcggcttctcgctggcgcaaaaaatggtaggccgtgcctgtggcgtgaaaggcattcgtccgggcgcgtactgtgaaccgaaaatgacttctgtaggttcccaggacaccaccggcccgatgacccgtgatgaactgaaagacctggcgtgcctgggcttctcggctgacctggtgatgcagtctttctgccacaccgcggcgtatccgaagccagttgacgtgaacacgcaccacacgctgccggacttcattatgaaccgtggcggtgtgtcgctgcgtccgggtgacggcgtcattcactcctggctgaaccgtatgctgctgccggataccgtcggtaccggtggtgactcccatacccgtttcccgatcggtatctctttcccggcgggttctggtctggtggcgtttgctgccgcaactggcgtaatgccgcttgatatgccggaatccgttctggtgcgcttcaaaggcaaaatgcagccgggcatcaccctgcgcgatctggtacacgctattccgctgtatgcgatcaaacaaggtctgctgaccgttgagaagaaaggcaagaaaaacatcttctctggccgcatcctggaaattgaaggtctgccggatctgaaagttgagcaggcctttgagctaaccgatgcgtccgccgagcgttctgccgctggttgtaccatcaagctgaacaaagaaccgatcatcgaatacctgaactctaacatcgtcctgctgaagtggatgatcgcggaaggttacggcgatcgtcgtaccctggaacgtcgtattcagggcatggaaaaatggctggcgaatcctgagctgctggaagccgatgcagatgcggaatacgcggcagtgatcgacatcgatctggcggatattaaagagccaatcctgtgtgctccgaacgacccggatgacgcgcgtccgctgtctgcggtacagggtgagaagatcgacgaagtgtttatcggttcctgcatgaccaacatcggtcacttccgtgctgcgggtaaactgctggatgcgcataaaggtcagttgccgacccgcctgtgggtggcaccgccaacccgtatggacgccgcacagttgaccgaagaaggctactacagcgtcttcggtaagagtggtgcgcgtatcgagatccctggctgttccctgtgtatgggtaaccaggcgcgtgtggcggacggtgcaacggtggtttccacctctacccgtaacttcccgaaccgtctgggtactggcgcgaatgtcttcctggcttctgcggaactggcggctgttgcggcgctgattggcaaactgccgacgccggaagagtaccagacctacgtggcgcaggtagataaaacagccgttgatacttaccgttatctgaacttcaaccagctttctcagtacaccgagaaagccgatggggtgattttccagactgcggtttaa

SEQ ID NO: 157 - protein, 891 aa, Escherichia coli
MSSTLREASKDTLQAKDKTYHYYSLPLAAKSLGDITRLPKSLKVLLENLLRWQDGNSVTEEDIHALAGWLKNAHADREIAYRPARVLMQDFTGVPAVVDLAAMREAVKRLGGDTAKVNPLSPVDLVIDHSVTVDRFGDDEAFEENVRLEMERNHERYVFLKWGKQAFSRFSVVPPGTGICHQVNLEYLGKAVWSELQDGEWIAYPDTLVGTDSHTTMINGLGVLGWGVGGIEAEAAMLGQPVSMLIPDVVGFKLTGKLREGITATDLVLTVTQMLRKHGVVGKFVEFYGDGLDSLPLADRATIANMSPEYGATCGFFPIDAVTLDYMRLSGRSEDQVELVEKYAKAQGMWRNPGDEPIFTSTLELDMNDVEASLAGPKRPQDRVALPDVPKAFAASNELEVNATHKDRQPVDYVMNGHQYQLPDGAVVIAAITSCTNTSNPSVLMAAGLLAKKAVTLGLKRQPWVKASLAPGSKVVSDYLAKAKLTPYLDELGFNLVGYGCTTCIGNSGPLPDPIETAIKKSDLTVGAVLSGNRNFEGRIHPLVKTNWLASPPLVVAYALAGNMNINLASEPIGHDRKGDPVYLKDIWPSAQEIARAVEQVSTEMFRKEYAEVFEGTAEWKGINVTRSDTYGWQEDSTYIRLSPFFDEMQATPAPVEDIHGARILAMLGDSVTTDHISPAGSIKPDSPAGRYLQGRGVERKDFNSYGSRRGNHEVMMRGTFANIRIRNEMVPGVEGGMTRHLPDSDVVSIYDAAMRYKQEQTPLAVIAGKEYGSGSSRDWAAKGPRLLGIRVVIAESFERIHRSNLIGMGILPLEFPQGVTRKTLGLTGEEKIDIGDLQNLQPGATVPVTLTRADGSQEVVPCRCRIDTATELTYYQNDGILHYVIRNMLK

SEQ ID NO: 158 - DNA, 2676 bp, Escherichia coli
atgtcgtcaaccctacgagaagccagtaaggacacgttgcaggccaaagataaaacttaccactactacagcctgccgcttgctgctaaatcactgggcgatatcacccgtctacccaagtcactcaaagttttgctcgaaaacctgctgcgctggcaggatggtaactcggttaccgaagaggatatccacgcgctggcaggatggctgaaaaatgcccatgctgaccgtgaaattgcctaccgcccggcaagggtgctgatgcaggactttaccggcgtacctgccgttgttgatctggcggcaatgcgcgaagcggttaaacgcctcggcggcgatactgcaaaggttaacccgctctcaccggtcgacctggtcattgaccactcggtgaccgtcgatcgttttggtgatgatgaggcatttgaagaaaacgtacgcctggaaatggagcgcaaccacgaacgttatgtgttcctgaaatggggaaagcaagcgttcagtcggtttagcgtcgtgccgccaggcacaggcatttgccatcaggttaacctcgaatatctcggcaaagcagtgtggagtgaattgcaggacggtgaatggattgcttatccggatacactcgttggtactgactcgcacaccaccatgatcaacggccttggcgtgctggggtggggcgttggtgggatcgaagcagaagccgcaatgttaggccagccggtttccatgcttatcccggatgtagtgggcttcaaacttaccggaaaattacgtgaaggtattaccgccacagacctggttctcactgttacccaaatgctgcgcaaacatggcgtggtggggaaattcgtcgaattttatggtgatggtctggattcactaccgttggcggatcgcgccaccattgccaatatgtcgccagaatatggtgccacctgtggcttcttcccaatcgatgctgtaaccctcgattacatgcgtttaagcgggcgcagcgaagatcaggtcgagttggtcgaaaaatatgccaaagcgcagggcatgtggcgtaacccgggcgatgaaccaatttttaccagtacgttagaactggatatgaatgacgttgaagcgagcctggcagggcctaaacgcccacaggatcgcgttgcactgcccgatgtaccaaaagcatttgccgccagtaacgaactggaagtgaatgccacgcataaagatcgccagccggtcgattatgttatgaacggacatcagtatcagttacctgatggcgctgtggtcattgctgcgataacctcgtgcaccaacacctctaacccaagtgtgctgatggccgcaggcttgctggcgaaaaaagccgtaactctgggcctcaagcggcaaccatgggtcaaagcgtcgctggcaccgggttcgaaagtcgtttctgattatctggcaaaagcgaaactgacaccgtatctcgacgaactggggtttaaccttgtgggatacggttgtaccacctgtattggtaactctgggccgctgcccgatcctatcgaaacggcaatcaaaaaaagcgatttaaccgtcggtgcggtgctgtccggcaaccgtaactttgaaggccgtatccatccgctggttaaaactaactggctggcctcgccgccgctggtggttgcctatgcgctggcgggaaatatgaatatcaacctggcttctgagcctatcggccatgatcgcaaaggcgatccggtttatctgaaagatatctggccatcggcacaagaaattgcccgtgcggtagaacaagtctccacagaaatgttccgcaaagagtacgcagaagtttttgaaggcacagcagagtggaagggaattaacgtcacacgatccgatacctacggttggcaggaggactcaacctatattcgcttatcgcctttctttgatgaaatgcaggcaacaccagcaccagtggaagatattcacggtgcgcggatcctcgcaatgctgggggattcagtcaccactgaccatatctctccggcgggcagtattaagcccgacagcccagcgggtcgatatctacaaggtcggggtgttgagcgaaaagactttaactcctacggttcgcggcgtggtaaccatgaagtgatgatgcgcggcaccttcgccaatattcgcatccgtaatgaaatggtgcctggcgttgaaggggggatgacgcggcatttacctgacagcgacgtagtctctatttatgatgctgcgatgcgctataagcaggagcaaacgccgctggcggtgattgccgggaaagagtatggatcaggctccagtcgtgactgggcggcaaaaggtccgcgtctgcttggtattcgtgtggtgattgccgaatcgtttgaacgaattcaccgttcgaatttaattggcatgggcatcctgccgctggaatttccgcaaggcgtaacgcgtaaaacgttagggctaaccggggaagagaagattgatattggcgatctgcaaaacctacaacccggcgcgacggttccggtgacgcttacgcgcgcggatggtagccaggaagtcgtaccctgccgttgtcgtatcgacaccgcgacggagttgacctactaccagaacgacggcattttgcattatgtcattcgtaatatgttgaagtaa

SEQ ID NO: 159 - protein, 416 aa, Escherichia coli
MESKVVVPAQGKKITLQNGKLNVPENPIIPYIEGDGIGVDVTPAMLKVVDAAVEKAYKGERKISWMEIYTGEKSTQVYGQDVWLPAETLDLIREYRVAIKGPLTTPVGGGIRSLNVALRQELDLYICLRPVRYYQGTPSPVKHPELTDMVIFRENSEDIYAGIEWKADSADAEKVIKFLREEMGVKKIRFPEHCGIGIKPCSEEGTKRLVRAAIEYAIANDRDSVTLVHKGNIMKFTEGAFKDWGYQLAREEFGGELIDGGPWLKVKNPNTGKEIVIKDVIADAFLQQILLRPAEYDVIACMNLNGDYISDALAAQVGGIGIAPGANIGDECALFEATHGTAPKYAGQDKVNPGSIILSAEMMLRHMGWTEAADLIVKGMEGAINAKTVTYDFERLMDGAKLLKCSEFGDAIIENM

SEQ ID NO: 160 - DNA, 1251 bp, Escherichia coli
atggaaagtaaagtagttgttccggcacaaggcaagaagatcaccctgcaaaacggcaaactcaacgttcctgaaaatccgattatcccttacattgaaggtgatggaatcggtgtagatgtaaccccagccatgctgaaagtggtcgacgctgcagtcgagaaagcctataaaggcgagcgtaaaatctcctggatggaaatttacaccggtgaaaaatccacacaggtttatggtcaggacgtctggctgcctgctgaaactcttgatctgattcgtgaatatcgcgttgccattaaaggtccgctgaccactccggttggtggcggtattcgctctctgaacgttgccctgcgccaggaactggatctctacatctgcctgcgtccggtacgttactatcagggcactccaagcccggttaaacaccctgaactgaccgatatggttatcttccgtgaaaactcggaagacatttatgcgggtatcgaatggaaagcagactctgccgacgccgagaaagtgattaaattcctgcgtgaagagatgggggtgaagaaaattcgcttcccggaacattgtggtatcggtattaagccgtgttcggaagaaggcaccaaacgtctggttcgtgcagcgatcgaatacgcaattgctaacgatcgtgactctgtgactctggtgcacaaaggcaacatcatgaagttcaccgaaggagcgtttaaagactggggctaccagctggcgcgtgaagagtttggcggtgaactgatcgacggtggcccgtggctgaaagttaaaaacccgaacactggcaaagagatcgtcattaaagacgtgattgctgatgcattcctgcaacagatcctgctgcgtccggctgaatatgatgttatcgcctgtatgaacctgaacggtgactacatttctgacgccctggcagcgcaggttggcggtatcggtatcgcccctggtgcaaacatcggtgacgaatgcgccctgtttgaagccacccacggtactgcgccgaaatatgccggtcaggacaaagtaaatcctggctctattattctctccgctgagatgatgctgcgccacatgggttggaccgaagcggctgacttaattgttaaaggtatggaaggcgcaatcaacgcgaaaaccgtaacctatgacttcgagcgtctgatggatggcgctaaactgctgaaatgttcagagtttggtgacgcgatcatcgaaaacatgtaa

SEQ ID NO: 161 - protein, 933 aa, Escherichia coli
MQNSALKAWLDSSYLSGANQSWIEQLYEDFLTDPDSVDANWRSTFQQLPGTGVKPDQFHSQTREYFRRLAKDASRYSSTISDPDTNVKQVKVLQLINAYRFRGHQHANLDPLGLWQQDKVADLDPSFHDLTEADFQETFNVGSFASGKETMKLGELLEALKQTYCGPIGAEYMHITSTEEKRWIQQRIESGRATFNSEEKKRFLSELTAAEGLERYLGAKFPGAKRFSLEGGDALIPMLKEMIRHAGNSGTREVVLGMAHRGRLNVLVNVLGKKPQDLFDEFAGKHKEHLGTGDVKYHMGFSSDFQTDGGLVHLALAFNPSHLEIVSPVVIGSVRARLDRLDEPSSNKVLPITIHGDAAVTGQGVVQETLNMSKARGYEVGGTVRIVINNQVGFTTSNPLDARSTPYCTDIGKMVQAPIFHVNADDPEAVAFVTRLALDFRNTFKRDVFIDLVCYRRHGHNEADEPSATQPLMYQKIKKHPTPRKIYADKLEQEKVATLEDATEMVNLYRDALDAGDCVVAEWRPMNMHSFTWSPYLNHEWDEEYPNKVEMKRLQELAKRISTVPEAVEMQSRVAKIYGDRQAMAAGEKLFDWGGAENLAYATLVDEGIPVRLSGEDSGRGTFFHRHAVIHNQSNGSTYTPLQHIHNGQGAFRVWDSVLSEEAVLAFEYGYATAEPRTLTIWEAQFGDFANGAQVVIDQFISSGEQKWGRMCGLVMLLPHGYEGQGPEHSSARLERYLQLCAEQNMQVCVPSTPAQVYHMLRRQALRGMRRPLVVMSPKSLLRHPLAVSSLEELANGTFLPAIGEIDELDPKGVKRVVMCSGKVYYDLLEQRRKNNQHDVAIVRIEQLYPFPHKAMQEVLQQFAHVKDFVWCQEEPLNQGAWYCSQHHFREVIPFGASLRYAGRPASASPAVGYMSVHQKQQQDLVNDALNVE

SEQ ID NO: 162 - DNA, 2802 bp, Escherichia coli
atgcagaacagcgctttgaaagcctggttggactcttcttacctctctggcgcaaaccagagctggatagaacagctctatgaagacttcttaaccgatcctgactcggttgacgctaactggcgttcgacgttccagcagttacctggtacgggagtcaaaccggatcaattccactctcaaacgcgtgaatatttccgccgcctggcgaaagacgcttcacgttactcttcaacgatctccgaccctgacaccaatgtgaagcaggttaaagtcctgcagctcattaacgcataccgcttccgtggtcaccagcatgcgaatctcgatccgctgggactgtggcagcaagataaagtggccgatctggatccgtctttccacgatctgaccgaagcagacttccaggagaccttcaacgtcggttcatttgccagcggcaaagaaaccatgaaactcggcgagctgctggaagccctcaagcaaacctactgcggcccgattggtgccgagtatatgcacattaccagcaccgaagaaaaacgctggatccaacagcgtatcgagtctggtcgcgcgactttcaatagcgaagagaaaaaacgcttcttaagcgaactgaccgccgctgaaggtcttgaacgttacctcggcgcaaaattccctggcgcaaaacgcttctcgctggaaggcggtgacgcgttaatcccgatgcttaaagagatgatccgccacgctggcaacagcggcacccgcgaagtggttctcgggatggcgcaccgtggtcgtctgaacgtgctggtgaacgtgctgggtaaaaaaccgcaagacttgttcgacgagttcgccggtaaacataaagaacacctcggcacgggtgacgtgaaataccacatgggcttctcgtctgacttccagaccgatggcggcctggtgcacctggcgctggcgtttaacccgtctcaccttgagattgtaagcccggtagttatcggttctgttcgtgcccgtctggacagacttgatgagccgagcagcaacaaagtgctgccaatcaccatccacggtgacgccgcagtgaccgggcagggcgtggttcaggaaaccctgaacatgtcgaaagcgcgtggttatgaagttggcggtacggtacgtatcgttatcaacaaccaggttggtttcaccacctctaatccgctggatgcccgttctacgccgtactgtactgatatcggtaagatggttcaggccccgattttccacgttaacgcggacgatccggaagccgttgcctttgtgacccgtctggcgctcgatttccgtaacacctttaaacgtgatgtcttcatcgacctggtgtgctaccgccgtcacggccacaacgaagccgacgagccgagcgcaacccagccgctgatgtatcagaaaatcaaaaaacatccgacaccgcgcaaaatctacgctgacaagctggagcaggaaaaagtggcgacgctggaagatgccaccgagatggttaacctgtaccgcgatgcgctggatgctggcgattgcgtagtggcagagtggcgtccgatgaacatgcactctttcacctggtcgccgtacctcaaccacgaatgggacgaagagtacccgaacaaagttgagatgaagcgcctgcaggagctggcgaaacgcatcagcacggtgccggaagcagttgaaatgcagtctcgcgttgccaagatttatggcgatcgccaggcgatggctgccggtgagaaactgttcgactggggcggtgcggaaaacctcgcttacgccacgctggttgatgaaggcattccggttcgcctgtcgggtgaagactccggtcgcggtaccttcttccaccgccacgcggtgatccacaaccagtctaacggttccacttacacgccgctgcaacatatccataacgggcagggcgcgttccgtgtctgggactccgtactgtctgaagaagcagtgctggcgtttgaatatggttatgccaccgcagaaccacgcactctgaccatctgggaagcgcagttcggtgacttcgccaacggtgcgcaggtggttatcgaccagttcatctcctctggcgaacagaaatggggccggatgtgtggtctggtgatgttgctgccgcacggttacgaagggcaggggccggagcactcctccgcgcgtctggaacgttatctgcaactttgtgctgagcaaaacatgcaggtttgcgtaccgtctaccccggcacaggtttaccacatgctgcgtcgtcaggcgctgcgcgggatgcgtcgtccgctggtcgtgatgtcgccgaaatccctgctgcgtcatccgctggcggtttccagcctcgaagaactggcgaacggcaccttcctgccagccatcggtgaaatcgacgagcttgatccgaagggcgtgaagcgcgtagtgatgtgttctggtaaggtttattacgacctgctggaacagcgtcgtaagaacaatcaacacgatgtcgccattgtgcgtatcgagcaactctacccgttcccgcataaagcgatgcaggaagtgttgcagcagtttgctcacgtcaaggattttgtctggtgccaggaagagccgctcaaccagggcgcatggtactgcagccagcatcatttccgtgaagtgattccgtttggggcttctctgcgttatgcaggccgcccggcctccgcctctccggcggtagggtatatgtccgttcaccagaaacagcaacaagatctggttaatgacgcgctgaacgtcgaataa

SEQ ID NO: 163 - protein, 405 aa, Escherichia coli
MSSVDILVPDLPESVADATVATWHKKPGDAVVRDEVLVEIETDKVVLEVPASADGILDAVLEDEGTTVTSRQILGRLREGNSAGKETSAKSEEKASTPAQRQQASLEEQNNDALSPAIRRLLAEHNLDASAIKGTGVGGRLTREDVEKHLAKAPAKESAPAAAAPAAQPALAARSEKRVPMTRLRKRVAERLLEAKNSTAMLTTFNEVNMKPIMDLRKQYGEAFEKRHGIRLGFMSFYVKAVVEALKRYPEVNASIDGDDVVYHNYFDVSMAVSTPRGLVTPVLRDVDTLGMADIEKKIKELAVKGRDGKLTVEDLTGGNFTITNGGVFGSLMSTPIINPPQSAILGMHAIKDRPMAVNGQVEILPMMYLALSYDHRLIDGRESVGFLVTIKELLEDPTRLLLDV

SEQ ID NO: 164 - DNA, 1218 bp, Escherichia coli
atgagtagcgtagatattctggtccctgacctgcctgaatccgtagccgatgccaccgtcgcaacctggcataaaaaacccggcgacgcagtcgtacgtgatgaagtgctggtagaaatcgaaactgacaaagtggtactggaagtaccggcatcagcagacggcattctggatgcggttctggaagatgaaggtacaacggtaacgtctcgtcagatccttggtcgcctgcgtgaaggcaacagcgccggtaaagaaaccagcgccaaatctgaagagaaagcgtccactccggcgcaacgccagcaggcgtctctggaagagcaaaacaacgatgcgttaagcccggcgatccgtcgcctgctggctgaacacaatctcgacgccagcgccattaaaggcaccggtgtgggtggtcgtctgactcgtgaagatgtggaaaaacatctggcgaaagccccggcgaaagagtctgctccggcagcggctgctccggcggcgcaaccggctctggctgcacgtagtgaaaaacgtgtcccgatgactcgcctgcgtaagcgtgtggcagagcgtctgctggaagcgaaaaactccaccgccatgctgaccacgttcaacgaagtcaacatgaagccgattatggatctgcgtaagcagtacggtgaagcgtttgaaaaacgccacggcatccgtctgggctttatgtccttctacgtgaaagcggtggttgaagccctgaaacgttacccggaagtgaacgcttctatcgacggcgatgacgtggtttaccacaactatttcgacgtcagcatggcggtttctacgccgcgcggcctggtgacgccggttctgcgtgatgtcgataccctcggcatggcagacatcgagaagaaaatcaaagagctggcagtcaaaggccgtgacggcaagctgaccgttgaagatctgaccggtggtaacttcaccatcaccaacggtggtgtgttcggttccctgatgtctacgccgatcatcaacccgccgcagagcgcaattctgggtatgcacgctatcaaagatcgtccgatggcggtgaatggtcaggttgagatcctgccgatgatgtacctggcgctgtcctacgatcaccgtctgatcgatggtcgcgaatccgtgggcttcctggtaacgatcaaagagttgctggaagatccgacgcgtctgctgctggacgtgtag

SEQ ID NO: 165 - protein, 474 aa, Escherichia coli [sequence omitted]

SEQ ID NO: 166 - DNA, 1425 bp, Escherichia coli; identical to SEQ ID NO: 24

SEQ ID NO: 167 - protein, 119 aa, Escherichia coli
MINPNPKRSDEPVFWGLFGAGGMWSAIIAPVMILLVGILLPLGLFPGDALSYERVLAFAQSFIGRVFLFLMIVLPLWCGLHRMHHAMHDLKIHVPAGKWVFYGLAAILTVVTLIGVVTI

SEQ ID NO: 168 - DNA, 360 bp, Escherichia coli
atgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaa

SEQ ID NO: 169 - protein, 131 aa, Escherichia coli
MTTKRKPYVRPMTSTWWKKLPFYRFYMLREGTAVPAVWFSIELIFGLFALKNGPEAWAGFVDFLQNPVIVIINLITLAAALLHTKTWFELAPKAANIIVKDEKMGPEPIIKSLWAVTVVATIVILFVALYW

SEQ ID NO: 170 - DNA, 396 bp, Escherichia coli
atgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaa

SEQ ID NO: 171 - protein, 244 aa, Escherichia coli
MAEMKNLKIEVVRYNPEVDTAPHSAFYEVPYDATTSLLDALGYIKDNLAPDLSYRWSCRMAICGSCGMMVNNVPKLACKTFLRDYTDGMKVEALANFPIERDLVVDMTHFIESLEAIKPYIIGNSRTADQGTNIQTPAQMAKYHQFSGCINCGLCYAACPQFGLNPEFIGPAAITLAHRYNEDSRDHGKKERMAQLNSQNGVWSCTFVGYCSEVCPKHVDPAAAIQQGKVESSKDFLIATLKPR

SEQ ID NO: 172 - DNA, 735 bp, Escherichia coli
atggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaa

SEQ ID NO: 173 - protein, 602 aa, Escherichia coli
MQTFQADLAIVGAGGAGLRAAIAAAQANPNAKIALISKVYPMRSHTVAAEGGSAAVAQDHDSFEYHFHDTVAGGDWLCEQDVVDYFVHHCPTEMTQLELWGCPWSRRPDGSVNVRRFGGMKIERTWFAADKTGFHMLHTLFQTSLQFPQIQRFDEHFVLDILVDDGHVRGLVAMNMMEGTLVQIRANAVVMATGGAGRVYRYNTNGGIVTGDGMGMALSHGVPLRDMEFVQYHPTGLPGSGILMTEGCRGEGGILVNKNGYRYLQDYGMGPETPLGEPKNKYMELGPRDKVSQAFWHEWRKGNTISTPRGDVVYLDLRHLGEKKLHERLPFICELAKAYVGVDPVKEPIPVRPTAHYTMGGIETDQNCETRIKGLFAVGECSSVGLHGANRLGSNSLAELVVFGRLAGEQATERAATAGNGNEAAIEAQAAGVEQRLKDLVNQDGGENWAKIRDEMGLAMEEGCGIYRTPELMQKTIDKLAELQERFKRVRITDTSSVFNTDLLYTIELGHGLNVAECMAHSAMARKESRGAHQRLDEGCTERDDVNFLKHTLAFRDADGTTRLEYSDVKITTLPPAKRVYGGEADAADKAEAANKKEKANG

SEQ ID NO: 174 - DNA, 1809 bp, Escherichia coli
gtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctga

SEQ ID NO: 175 - protein, 153 aa, Propionibacterium acidipropionici
MENFNNDPFACIDHVGYAVKDMDEAIKYHTEVLGFHVLLREKNEGHGVEEAMIATGKRGEESTVVQLLAPLGEDTTIGKYLAKNKNMIQQVCYRTYDIDKTIATLKERGARFTSEEPSSGTAGSRVIFLHPKYTGGLLIEITEPPAGGMPYKD

SEQ ID NO: 176 - DNA, 462 bp, Propionibacterium acidipropionici
atggagaacttcaacaacgatcctttcgcgtgtatcgatcacgtcggctacgcggtcaaggacatggacgaggccatcaagtatcacaccgaggtgctcggcttccacgtgctgctgcgtgagaagaacgagggtcacggcgtcgaggaggcgatgatcgccaccggcaagcgcggcgaggagagcaccgtcgtccagctgctcgcccccctcggcgaggacaccaccatcggcaagtacctggccaagaacaagaacatgatccagcaggtgtgctaccgcacctacgacatcgacaagaccatcgcgaccctcaaggagcgcggggccaggttcacctccgaggagccctcctccggcaccgccgggtcccgggtcatcttcctccacccgaagtacaccggcggtctgctcatcgagatcaccgagcccccggccggcggcatgccctacaaggactga